(19) United States
(12) Patent Application Publication
(10) Pub. No.: US 2025/0302972 A1
(43) Pub. Date: Oct. 2, 2025

(54) FORMULATION

(71) Applicant: ENA Respiratory Pty Ltd, Sydney (AU)

(72) Inventor: Grant McLachlan, Melbourne (AU)

(21) Appl. No.: 18/688,369

(22) PCT Filed: Sep. 2, 2022
(86) PCT No.: PCT/AU2022/051074

§ 371 Date: Mar. 1, 2024

(30) Foreign Application Priority Data
Sep. 2, 2021 (AU) 2021902855

(51) Int. Cl. A61K 47/60; A61K 9/14; A61K 31/23; A61K 31/724; A61K 38/00

(52) U.S. Cl. CPC A61K 47/60; A61K 9/14; A61K 31/23; A61K 31/724; A61K 38/00

(57) ABSTRACT

This invention relates to pharmaceutical compositions comprising a compound comprising a TLR2 agonist moiety conjugated with a solubilising moiety and an excipient that stabilises the compound under accelerated aging conditions. Also disclosed is the preparation of powdered forms of the compositions and methods for their use.

### CROSS-REFERENCE TO RELATED APPLICATIONS

This application is a 35 U.S.C. § 371 national stage filing of International Application No. PCT/AU2022/051074, filed on Sep. 2, 2022, which claims priority to AU Patent Application No. 2021902855, filed Sep. 2, 2021. The entire contents of each of the above applications are incorporated herein by reference.

This application claims priority to Australian provisional patent application no. 2021902855 filed on 2 Sep. 2021, the entire contents of which are incorporated herein by reference.

### FIELD OF THE INVENTION

This invention relates to a pharmaceutical composition comprising a Toll-Like Receptor 2 protein (TLR2) agonist.

### BACKGROUND OF THE INVENTION

TLR2 agonists have previously been identified to show potential in treating respiratory diseases and conditions associated with infectious agents such as viruses and bacteria. Respiratory infections are among the most common causes of human disease worldwide and are commonly caused by viruses. According to the World Health Organisation (WHO), worldwide, seasonal epidemics of influenza alone are estimated to result in about 3 to 5 million cases of severe illness, and about 250,000 to 500,000 deaths per year.

Although vaccines are available for some seasonal strains, for example influenza, these have not always been shown to be adequate due to several factors, such as infection between the lag phase between inoculation and the formation of antibodies and immune cells being formed. Seasonal vaccinations often also need modification, including re-formulation and administration, and may also not provide protection for the full length of time desired. For other occurrences of influenza, such as unexpected pandemic outbreaks, a vaccine is not always known, developed or available.

Viral respiratory infections can also worsen the severity of diseases of respiratory conditions leading to exacerbations (attacks). Exacerbations can occur for conditions such as asthma and chronic obstructive pulmonary disease (COPD). Asthma and COPD exacerbations are the most clinically and economically important forms of the diseases.

The vast majority of exacerbations, particularly in asthma, continue to occur despite use of the best available current therapies. When exacerbations do occur, treatment options are limited. Existing approved treatment involves increasing doses of inhaled bronchodilators and systemic or oral corticosteroids—which are the same drugs that failed to prevent the exacerbation occurring in the first place.

There is a need, therefore, for new or improved compositions comprising TLR2 agonists that are in a form suitable for administration. Advantageously, the TLR2 compositions will be shelf-stable for extended periods and possess storage stability under ambient conditions.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention relates to compositions of TLR2 agonists that are suitable for administration to a subject. The compositions comprise compounds comprising a TLR2 agonist moiety conjugated with a solubilising moiety. While the conjugation of a TLR2 agonist moiety with a solubilising moiety increases the solubility, and hence bioavailability, the inventors have found that formulating these compounds with some excipients led to a loss of stability rendering the formulations not sufficiently stable for commercial use. Surprisingly, the inventors have found formulations of these compounds that provide the compound in a form that does not suffer from significant losses of stability for a range of administration routes.

In a first aspect, the invention provides a pharmaceutical composition comprising a cyclodextrin and a compound comprising a TLR2 agonist moiety conjugated with a solubilising moiety.

In a second aspect, the invention provides a pharmaceutical composition comprising a compound comprising a TLR2 agonist moiety conjugated with a solubilising moiety and a sugar compound selected from mannitol, erythritol, xylitol, sorbitol, myo-inositol or a combination thereof.

In any pharmaceutical composition described herein, the compound comprising a TLR2 agonist moiety conjugated with a solubilising moiety may be provided in the form of a pharmaceutically acceptable salt, solvate, stereoisomer or prodrug.

In any aspect of the invention, the pharmaceutical compositions may comprise any compound described herein. Preferably, the compound may be as defined by any one of formulas (I), (IA1), (IA2), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII) and (XXIII) (collectively referred to herein as formulas (I)-(XXIII)).

In any aspect of the invention, the compound may comprise a TLR2 agonist moiety A selected from A1′ and A2 as defined herein and a polyethylene glycol (PEG), wherein the moiety A and PEG are covalently linked by a glycine, serine, homoserine, threonine, phosphoserine, asparagine or glutamine residue, or an ester of a glutamine residue.

In any aspect of the invention, the compound may comprise or consist of partial structure A1Y′ or A2Y′:

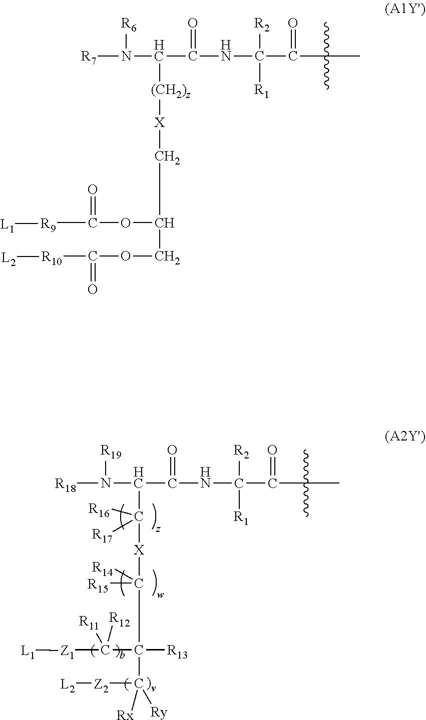

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
b and w are each independently an integer from 0 to 7 and v is an integer from 0 to 5, provided that:
the sum of b, v, and w is at least 3; and
the sum of b and w is from 0 to 7;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₈ aliphatic;
R₁₉ is H, C₁-C₆ aliphatic, an amino protecting group, L₃-C(═O)—, or A2;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈,
R₁₉, Rₓ, R_{y}, L₁, L₂, and L₃ is optionally substituted; and
A1Y′ or A2Y′ is covalently linked to polyethylene glycol (PEG),
or a pharmaceutically acceptable salt, solvate or prodrug thereof.

In some embodiments, the compound may be selected from any of compounds 001-010, A101-A118, A201-A232 and B1-B16.

Reference to a “compound of the invention” as used herein may refer to any of:
a compound of formulas (I)-(XXIII);
a compound selected from any of compounds 001-010, A101-A118, A201-A232 and B1-B16;
a compound comprising moiety A selected from A1′ and A2 as defined herein and a polyethylene glycol (PEG), wherein the moiety A and PEG are linked by a glycine, serine, homoserine, threonine, phosphoserine, asparagine or glutamine residue, or an ester of a glutamine residue; and/or
a compound comprising a partial structure of formula (A1Y′) or (A2Y′) covalently linked to a PEG.

Compounds comprising a TLR2 agonist moiety conjugated with a solubilising moiety (including their synthesis) have been described in WO 2018/176099 (US 2020/0147028 A1), WO 2019/119067 (US 2021/0230217 A1), WO 2020/257870 and international application no. PCT/AU2021/050667. The entire contents of each of these applications are incorporated herein by reference. Any of the TLR2 agonist compounds, or pharmaceutically acceptable salts, solvates, stereoisomers and/or prodrugs thereof, described in these documents may be included in a pharmaceutical composition described herein.

In another aspect, there is provided a compound of the invention, or a pharmaceutically acceptable salt, solvate, stereoisomer or prodrug thereof.

In a further aspect, the invention provides a method of preparing a powder comprising a compound comprising a TLR2 agonist moiety conjugated with a solubilising moiety, or a pharmaceutical acceptable salt, solvate, stereoisomer or prodrug thereof, and a cyclodextrin, the method comprising:
forming a solution comprising the compound and the cyclodextrin; and
spray drying the solution to provide the powder.

In a further aspect, the invention provides a pharmaceutical composition in the form of a powder prepared by these methods of the invention.

In another aspect, the invention provides a method of treating and/or preventing a disease, comprising raising an innate immune response in a subject by administering an effective amount of a pharmaceutical composition of the invention to the subject in need thereof.

In a further aspect, the invention provides a method of treating and/or preventing a disease caused by an infectious agent, comprising administering to a subject in need thereof an effective amount of a pharmaceutical composition of the invention.

In another aspect, the invention provides a method of treating and/or preventing a respiratory disease or condition associated with a viral or bacterial infection, comprising administering to a subject in need thereof a pharmaceutical composition of the invention.

In a further aspect, the invention provides a method of treating and/or preventing a respiratory infection, comprising administering to a subject in need thereof a pharmaceutical composition of the invention.

In another aspect, the invention provides a method for reducing airway inflammation, comprising administering to a subject in need thereof a pharmaceutical composition of the invention.

In another aspect, the invention provides a method of improving the ability of a subject to control a respiratory disease or condition during a respiratory viral infection, the method comprising administering to a subject in need thereof a pharmaceutical composition of the invention.

In a further aspect, the invention provides a method of treating and/or preventing a disease or condition associated with the TLR2 receptor, the method comprising administering to a subject in need thereof a pharmaceutical composition of the invention.

In another aspect, the invention provides use of a compound comprising a TLR2 agonist moiety conjugated with a solubilising moiety and/or a cyclodextrin in the manufacture of a medicament.

In another aspect, the invention provides a pharmaceutical composition of the invention for use as a medicament.

In a further aspect, the invention provides use of a compound comprising a TLR2 agonist moiety conjugated with a solubilising moiety and/or a sugar compound selected from mannitol, erythritol, xylitol, sorbitol, myo-inositol or a combination thereof in the manufacture of a medicament.

In some embodiments, the pharmaceutical compositions and/or medicaments are for one or more of the following:
treating and/or preventing a disease caused by an infectious agent;
treating and/or preventing a disease, comprising raising an innate immune response;
treating and/or preventing a respiratory disease or condition associated with a viral or bacterial infection;
treating and/or preventing a respiratory infection;
reducing airway inflammation;
improving the ability of a subject to control a respiratory disease or condition during a respiratory viral infection; and
treating and/or preventing a disease or condition associated with the TLR2 receptor.

In these methods, it may be advantageous to achieve local nasal administration of the compound comprising the TLR2 agonist moiety conjugated with the solubilising moiety.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Human TLR2 dose response (optical density at 650 nm vs concentration (ng/mL) of compound B6 from the NK-κB luciferase assay described in Example 9.

FIG. 2. Human TLR2 dose response (optical density at 650 nm vs concentration (ng/mL) of compound B12 from the NK-κB luciferase assay described in Example 9.

FIG. 3. Human TLR2 dose response (optical density at 650 nm vs concentration (ng/mL) of compound B4. B6, B8, B12, B14 and B16 and heat killed Listeria monocytogenes (HKLC) from the assay described in Example 10.

FIG. 4. TLR2 activity of compounds 4 of WO2020/257870 (A204) and B12 from the NK-κB luciferase assay described in Example 11.

FIG. 5. Percentage recovery from 3 or 6 week storage of compounds 4 of WO2020/257870 (A204), B12, B14 and B16 at 40° C. described in Example 12.

FIG. 6. (a) Weight loss of hamsters treated with A204 or PBS prior to infection and/or 8 hours post infection. Hamsters were intranasally treated with 50 ug/ml in 100 ul PBS 24 hours prior to infection or 8 hours post infection with 5×10⁴ PFU SARS-CoV-2 in 100 ul PBS. (b) RT-qPCR data quantifying lung viral load as assessed by N RNA copy number per ug total lung RNA.

FIG. 7. Weight loss of hamsters treated with 10 ug/ml A204 in 100 ul or PBS after infection with SARS-Cov-2. Hamsters were intranasally infected with 10⁴ PFU SARS-CoV-2 in 100 ul PBS and treated with A204 or PBS 8 hours post infection. Weights were measured 7-Days post infection.

FIG. 8. Interleukin-6 (IL-6) concentration in (A) nasal sampling and (B) serum taken from Cynomolgus Macaques following nasal administration with the Dry Powder (70:30 mannitol:hydroxypropyl methyl cellulose (HPMC)), the Dry Powder & A204 and the liquid spray solution formulations as described in Example 18.

FIG. 9. Interleukin-6 (IL-6) concentration in (A) nasal sampling and (B) serum taken from Cynomolgus Macaques following nasal administration with Dry Powder including β-cyclodextrin, Dry Powder & A204 and the liquid spray solution formulations as described in Example 18.

FIG. 10. Interleukin-6 (IL-6) concentration in (A) nasal sampling and (B) serum taken from Cynomolgus Macaques following nasal administration with Dry Powder & hydroxypropyl-β-cyclodextrin, Dry Powder & hydroxypropyl-β-cyclodextrin and the liquid spray solution formulations as described in Example 18.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The invention relates to a pharmaceutical composition comprising a compound comprising a TLR2 agonist moiety conjugated with a solubilising moiety. Preferably the solubilising moiety comprises a polyethyleneglycol (PEG). The compound is formulated with either (1) a cyclodextrin or (2) a sugar compound selected from mannitol, erythritol, xylitol, sorbitol, myo-inositol or a combination thereof.

When developing a formulation for administration of a compound of the invention to a subject, the inventors found that some excipients resulted in poor stability of the compound of the invention under accelerated storage conditions. The accelerated storage conditions typically involve elevated temperature and relative humidity (RH), for example, 25° C./60% RH or 40° C./70% RH. Surprisingly, the inventors found that formulating a compound of the invention with either of the inventive excipients resulted in improved stability in both solid (eg powder) and liquid (eg solution) states.

The pharmaceutical composition may be presented in any suitable form. Typically the TLR2 agonist compounds of the invention are administered in one or more of the following routes: intranasally, by inhalation, to the respiratory tract, the upper airway, the lower airway, both the upper and lower airway, intravenously, or any combination thereof. The pharmaceutical compositions may therefore be presented in solid or liquid form. Preferred solid forms include a powder, for example, a powder suitable for inhalation. Suitable powders may be a freeze-dried powder or a spray-dried powder. Spray-dried powders are preferred due to the potential for larger scale production runs. Preferred liquid forms include aqueous solutions suitable for intravenous (IV) administration.

In some embodiments, the pharmaceutical composition is in the form of a powder. The powder may be in a form suitable for intranasal administration. In some embodiments, the powder may have a minimum X₅₀ of at least about 5 μm, 10 μm, 15 μm, 20 μm, 30 μm, 40 μm, 45 μm, 50 μm, 55 μm or 60 μm. The maximum X₅₀ may be not more than about 80 μm, 70 μm, 60 μm, 50 μm, 40 μm, 35 μm or 30 μm. The X₅₀ may be from any of these minimum values to any maximum value, provided the minimum value is less than the maximum value. For example, the powder may have an X₅₀ from about 15 μm to about 80 μm or about 20 μm to about 50 μm. X₅₀ of the powder represents the size mesh that 50% of the particles present in the sample are able to pass through, thus the X₅₀ value defines the 50^{th} percentile for particle size in the sample.

In some embodiments, not more than 10% of particles have a particle size below about 10 μm. In these embodiments, the X₅₀ may be any X₅₀ values described herein.

Particle size typically refers to particles able to pass through a mesh graded to have pores of a particular particle size. The longest dimension of some particles passing the mesh may be greater than the specified particle size. The particle size however can be determined in a number of ways, for example, by passing particles through a series of meshes of different size and determining the mass of particles retained by each known mesh size, or by other more direct methods including laser analysis. Preferably, the particle sizes described herein may be determined by laser particle size analysis, for example by the method described in the Examples (see Example 13, subsection 13.1.2.5).

In some embodiments, the pharmaceutical composition comprises the compound in a minimum concentration of at least about 0.0001 wt %, 0.0005 wt %, 0.001 wt %, 0.005 wt %, 0.01 wt %, 0.05 wt %, 0.1 wt %, 0.5 wt %, 0.75 wt %, or 1 wt %. The maximum concentration of the compound may be not more than about 20 wt %, 15 wt %, 10 wt %, 9 wt %, 8 wt %, 7 wt %, 6 wt %, 5 wt %, 4 wt %, 3 wt %, 2 wt % or 1 wt %. The concentration of compound may be from any of these minimum concentrations to any of these maximum concentrations on the proviso that the minimum concentration is less than the maximum concentration. For example, in some embodiments, the pharmaceutical compositions comprise the compound in a concentration from about 0.0001 wt % to about 20 wt %, about 0.001 wt % to about 10 wt %, about 0.001 wt % to about 5 wt %, or about 0.1 wt % to about 2 wt %.

In embodiments where the pharmaceutical composition is in liquid form, the concentration of the compound may be any of the above minimum concentrations, maximum concentrations and ranges thereof, for example from about 0.0001 wt % to about 10 wt %, about 0.01 to about 2 wt % or about 0.01 wt % to about 1.5 wt %. In some embodiments where the pharmaceutical composition is in liquid form, the concentration of the compound may be about 0.75 wt %.

In embodiments where the pharmaceutical composition is in solid form, the concentration of the compound may be any of the above minimum concentrations, maximum concentrations and ranges thereof. Typically the concentration for solid form compositions may be higher than for liquid form compositions, particularly in solid form compositions intended for dispersion in a liquid carrier prior to administration. For example, the concentration of the compound in some solid form embodiments may be from about 0.0005 wt % to about 15 wt %, about 0.05 to about 5 wt %, about 0.05 wt % to about 2 wt %, about 0.5 wt % to about 5 wt % or about 1 wt % to about 5 wt %. In some embodiments where the pharmaceutical composition is in solid form, the concentration of the compound may be about 1 wt %.

### Cyclodextrin

In some embodiments, the pharmaceutical compositions comprise a cyclodextrin.

Any suitable cyclodextrin may be included in the composition. Suitable cyclodextrins include α-cyclodextrin, β-cyclodextrin, γ-cyclodextrin, and a cyclodextrin derivative of α-cyclodextrin, β-cyclodextrin or γ-cyclodextrin, and combinations thereof.

Cyclodextrin derivatives include hydroxypropyl-β-cyclodextrin, sulfobutylether-β-cyclodextrin, methyl-β-cyclodextrin, dimethyl-β-cyclodextrin, and randomly methylated-β-cyclodextrin, and combinations thereof.

In some embodiments, the pharmaceutical composition comprises β-cyclodextrin or a cyclodextrin derivative thereof, such as hydroxypropyl-β-cyclodextrin, sulfobutylether-β-cyclodextrin, methyl-β-cyclodextrin, dimethyl-β-cyclodextrin, and randomly methylated-β-cyclodextrin.

In some embodiments, the pharmaceutical composition comprises R-cyclodextrin.

In some embodiments, the pharmaceutical composition comprises hydroxypropyl-β-cyclodextrin.

The pharmaceutical composition may comprise the cyclodextrin in a stabilising amount. The stabilising amount may be any amount sufficient to provide a stable formulation of the stabilising the compound. Preferably, the stabilising amount of the cyclodextrin present in the compositions of the invention is less than an amount of cyclodextrin able to act as a permeation enhancer for the compound.

In some embodiments, the minimum ratio by weight of the compound to the cyclodextrin may be at least about 1:0.1, 1:0.2, 1:0.3, 1:0.4, 1:0.5, 1:0.6, 1:0.7, 1:0.8, 1:0.9, 1:1, 1:1.5, 1:2, 1:2.5, 1:3, 1:3.5, 1:4, 1:4.5, 1:5, 1:10, 1:20, 1:30, 1:40, 1:45, 1:49, 1:50, 1:51, 1:55, 1:60, 1:70, 1:80, 1:85, 1:90, 1:95, 1:98, 1:99. The maximum ratio by weight of the compound to the cyclodextrin may be not more than about 1:100, 1:99.9, 1:99.5, 1:99, 1:98, 1:95, 1:80, 1:70, 1:60, 1:55, 1:51, 1:50, 1:49, 1:45, 1:40, 1:30, 1:25, 1:20, 1:19, 1:18, 1:17, 1:16, 1:15, 1:14, 1:13, 1:12, 1:10, 1:9, 1:8, 1:7, 1:6, 1:6, 1:5.5, 1:5, 1:4, 1:3, 1:2 or 1:1. The ratio by weight of the compound to the cyclodextrin may be from any of these minimum ratios to any of these maximum ratios, with the proviso that the minimum ratio is less than the maximum ratio. For example, in some embodiments, the ratio by weight of the compound to the cyclodextrin may be from about 1:0.1 to about 1:100, about 1:0.1 to about 1:20, about 1:0.1 to about 1:1 or about 1:1 to about 1:20.

In some embodiments, the pharmaceutical composition comprising a cyclodextrin may comprise further pharmaceutically acceptable excipient(s). The further pharmaceutically acceptable excipient may be any compatible component. Examples of components are described in Martindale—The Extra Pharmacopoeia (Pharmaceutical Press, London 1993) and Martin (ed.), Remington's Pharmaceutical Sciences. In some embodiments, the pharmaceutical composition is a spray dried powder. Various spray dried compositions are reviewed in Alhajj, N. et al. Powder Technology 384 (2021) 313-331, which is entirely incorporated herein by reference. In such embodiments, the compositions may be comprise any excipient known to be compatible with the spray drying process. Suitable excipience include those described in Alhajj, N. et al. Powder Technology 384 (2021) 313-331.

In some embodiments, the further pharmaceutically acceptable excipient may comprise a sugar compound selected from mannitol, erythritol, xylitol, sorbitol, myo-inositol or a combination thereof. Preferably, the mannitol is pyrogen free grade mannitol. The combination of a compound of the invention, a cyclodextrin and these selected sugar compounds has been found to retain acceptable stability of the compound of the invention in the formulation.

In some embodiments, the further pharmaceutically acceptable excipient comprises a polymer. The polymer may be a viscosity modifying polymer. Any suitable polymer may be included. Suitable polymers include polysaccharides (such as dextran, hyaluronic acid, chitosan and alginic acid), proteins (such as albumin and gelatin), polyethylene glycol (PEG; such as PEG200, PEG300, PEG400, PEG600, PEG1000, PEG4000, PEG10000), poly vinyl alcohol (PVA), polyesters (such as poly lactic acid (PLA), poly glycolic acid (PGA), poly L-lactide co-glycolide (PLGA) co-polymer), polyvinyl pyrrolidone (PVP; povidone; such as PVP K17, PVP K25, PVP K30, PVP K90) and poly E caprolactone (PCL). In some embodiments, the polymer may be a polysaccharide. The polysaccharide may be pectin, cellulose, a cellulose derivative or a combination thereof. Suitable cellulose derivatives include methylcellulose (MC), hydroxypropylmethylcellulose (HPMC), hydroxyethylcellulose (HEC), hydroxypropylcellulose (HPC) and combinations thereof.

In some embodiments, the pharmaceutical composition comprises a polymer selected from pectin, methylcellulose, hydroxypropylmethyl cellulose, hydroxyethylcellulose, hydroxypropylcellulose or a combination thereof.

In some embodiments, the polymer may be selected from pectin, methylcellulose, hydroxypropylmethyl cellulose, hydroxyethylcellulose, or a combination thereof. For example, in these embodiments, the cyclodextrin may be hydroxypropyl-β-cyclodextrin.

In some embodiments, the polymer may be selected from pectin, hydroxypropylmethyl cellulose, methylcellulose, hydroxypropylmethyl cellulose, or a combination thereof. For example, in these embodiments, the cyclodextrin may be 3-cyclodextrin.

In some embodiments, the polymer may be selected from pectin, methylcellulose, or a combination thereof.

In some embodiments the pharmaceutical composition is substantially free from one or more of HEC, HPMC and/or HPC.

In some embodiments, the polymer may be a polyvinyl pyrrolidone.

The polymer may be present in any suitable amount. The amount will depend on the polymer selected as well as the other combination of components included in the composition. In some embodiments, the polymer may be present in an amount of up to about 20 wt %, 15 wt %, 10 wt %, 7.5 wt %, 5 wt %, 2.5 wt %, 1 wt %, 0.5 wt %, 0.1 wt %, or 0.01 wt %. In some embodiments, the amount of polymer may be from 0 wt % to any of these amounts, for example from 0 wt % to about 20 wt %, or between any of the above-mentioned amounts, such as from about 0.1 wt % to about 15 wt %.

In some embodiments, the pharmaceutical composition comprises, or consists of, the compound, the cyclodextrin, a sugar compound and a polymer.

In some embodiments, the pharmaceutical composition comprises a surface modification agent. One or more surface modification agents may be beneficial as an excipient(s) particularly in spray-dried solid form compositions. Without wishing to be bound by theory, it is believed that the surface modification agent may form a protective shell around the particle as it forms in the spray drying process. However, some embodiments of liquid form pharmaceutical compositions described herein may also comprise as an excipient any of the surface modification agents described herein. Suitable surface modification agents include amino acids (such as leucine) stearate salts (such as sodium stearate) phosphatidylcholines (PC). In some embodiments, the pharmaceutical composition comprises leucine, typically L-leucine. The leucine may be provided in any suitable form, including pharmaceutically acceptable salts and solvates thereof. The use of leucine as a surface modification agent in spray dried solid compositions is reviewed in Alhajj, N. et al. Drug Discovery Today, 26(10) (2021) 2384, which is entirely incorporated herein by reference.

The surface modification agent may be included in any suitable amount, where typically the maximum concentration of leucine is limited by its solubility in a liquid composition, or in a solution that is subsequently spray dried. The amount will depend on the agent selected. In some embodiments, the compositions may comprise up to about 20 wt %, 15 wt %, 12.5 wt %, 10 wt %, 9 wt %, 8 wt %, 7 wt %, 6 wt %, 5 wt %, 2.5 wt %, 1 wt %, 0.5 wt %, 0.1 wt %, 0.01 wt % or 0.001 wt %. In some embodiments, the amount of surface modification agent may be from 0 wt % to any of these amounts, for example from 0 wt % to about 20 wt %, or between any of the above-mentioned amounts, such as from about 0.1 wt % to about 15 wt % pr about 8 wt % to about 10 wt %.

In some embodiments, the pharmaceutical composition comprises a liquid carrier. The liquid carrier may be any pharmaceutically acceptable solvent. Typically the solvent is water optionally comprising a buffer or other electrolyte(s). Suitable buffers include phosphate buffered saline.

In some embodiments, the pharmaceutical composition comprises a pharmaceutically acceptable chelator. Suitable chelators include ethylenediamine tetraacetic acid (EDTA).

Each further pharmaceutically acceptable excipient may be included in any suitable amount, including traditional amounts for such excipients known in the art provided that the compound retains its stability in the final formulation. Typically, the minimum ratio by weight of the compound to each further pharmaceutically acceptable excipient may be at least about 1:0.1, 1:0.5, 1:1, 1:2, 1:3, 1:4, 1:5 or 1:10. The maximum ratio by weight of the compound to each further pharmaceutically acceptable excipient may be not more than about 1:20, 1:15, 1:10, 1:8, 1:7, 1:5, 1:3, 1:2 or 1:1. The ratio by weight of the compound to each further pharmaceutically acceptable excipient may be from any of these minimum ratios to any of these maximum ratios, for example, from about 1:0.1 to about 1:20 or about 1:1 to about 1:10. In some embodiments, each further excipient is included in the pharmaceutical composition in a ratio by weight of 1:5 (compound:excipient).

### Sugar Compound

In some embodiments, the pharmaceutical compositions comprise a sugar compound selected from mannitol, erythritol, xylitol, sorbitol, myo-inositol or a combination thereof. The mannitol included in these compositions is preferably pyrogen free grade mannitol.

Surprisingly, formulation of a compound of the invention with some sugar compounds resulted in a loss of stability of the compound under accelerated aging conditions, for example, inclusion of adonitol resulted in significant losses in concentration of a compound of the invention, while formulation of the compound with one of these specified sugar compounds resulted in improved stability of the compound over 3 weeks storage at accelerated aging conditions (40° C./70% RH).

In some embodiments, the minimum ratio by weight of the compound to the specified sugar compound may be at least about 1:0.1, 1:0.2, 1:0.3, 1:0.4, 1:0.5, 1:0.6, 1:0.7, 1:0.8, 1:0.9, 1:1, 1:1.5, 1:2, 1:2.5, 1:3, 1:3.5, 1:4, 1:4.5 or 1:5. The maximum ratio by weight of the compound to the specified sugar compound may be not more than about 1:20, 1:19, 1:18, 1:17, 1:16, 1:15, 1:14, 1:13, 1:12, 1:10, 1:9, 1:8, 1:7, 1:6, 1:6, 1:5.5, 1:5, 1:4, 1:3, 1:2 or 1:1. The ratio by weight of the compound to the specified sugar compound may be from any of these minimum ratios to any of these maximum ratios, with the proviso that the minimum ratio is less than the maximum ratio. For example, in some embodiments, the ratio by weight of the compound to the specified sugar compound may be from about 1:0.1 to about 1:20, about 1:0.1 to about 1:1 or about 1:1 to about 1:20.

In some embodiments, the pharmaceutical composition comprising the compound and the monosaccharaide is in the form of a freeze-dried powder.

Compound Comprising a TLR2 Agonist Moiety Conjugated with a Solubilising Moiety (Compound of the Invention)

The TLR2 agonist moiety may be any moiety capable of binding TLR2 and agonising its activity. Various TLR2 agonist moieties are described in the art. In some embodiments, the TLR2 agonist moiety is as included in any compound of the invention described herein.

In some embodiments, the TLR2 agonist moiety is selective for a heterodimer comprising TLR2. In some embodiments, the TLR2 agonist moiety is selective for a TLR2/TLR6 dimer. Thus, a selective TLR2 agonist moiety may have greater activity for the TLR2/TLR6 dimer over other TLR2 heteromers.

In some embodiments, the TLR2 agonist moiety may comprise a lipid, a peptidoglycan, a lipopeptide, a lipoprotein or a lipopolysaccharide capable of binding TLR2. Preferably, the TLR2 agonist moiety comprises at least one palmitoyl, myristoyl, stearoyl, lauroyl, octanoyl, or decanoyl group. The TLR2 agonist moiety may be selected from the group consisting of: Pam2Cys, Pam3Cys, Ste2Cys, Lau2Cys, and Oct2Cys. In a preferred embodiment, the TLR2 agonist comprises Pam2Cys.

The TLR2 agonist moiety is conjugated (eg covalently linked) with one or more functional groups that act to increase the solubility of the TLR2 agonist. As will be understood by persons skilled in the art, TLR2 agonists are typically non-polar and, accordingly, while being soluble in non-polar solvents, are only less soluble in polar and aqueous solvents.

A solubilising moiety may include one, or more than one, hydrophilic functional group(s) conjugated to TLR2 agonist in order to improve the solubility of the TLR2 agonist moiety. The solubilising moiety will generally be a polar moiety which increases the solubility of the TLR2 agonist moiety in polar or aqueous solvents.

In any aspect of the invention, the solubilising moiety may be a positively charged group. Positively charged groups of the present invention include but are not limited to penetratin, HIV Tat 48-60, HIV Rev 34-50, transportan, oligoarginine peptides (linear and branched), oligolysine peptides, pyrrrochoricin, alpha-helical amphipathic model peptide, polylysine, protamine, FL17, Magnafloc 1697, and the polycationic compounds described in U.S. Pat. Nos. 6,689,478 and 4,035,558.

In yet a further embodiment of the present invention, the solubilising moiety comprises, consists essentially of, or consists of a linear or branched peptide. Typically, the linear or branched peptide contains positively or negatively charged amino acids. Positively charged amino acids may be lysine, arginine, histidine, ornithine or combinations thereof. The branched or linear peptide may contain at least one lysine or arginine residue. Preferably, the charged amino acids are terminal, for example N-terminal. The branched peptides may have one of the following structures.

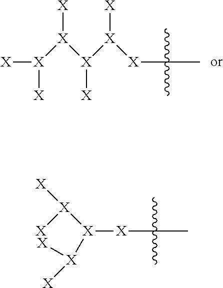

In the above structures X may independently be a charged residue, either a positively or negatively charged residue. Preferably the positively charged amino acids are lysine, arginine, histidine or ornithine. Preferably, the negatively charged amino acids are glutamate or aspartate.

As used herein, ‘PEG’ refers to the polymer compound polyethylene glycol. Unless otherwise defined, reference to ‘PEG’ includes any length polymer of ethylene oxide. Reference to PEG also includes substituted PEG.

The solubilising moiety may be one or more of the group consisting of “PEG” (or polyethyleneglycol) and a polar polypeptide such as “R4”, a hyper-branched tetra arginine complex; “H4”, a hyper-branched tetra histidine complex; “H8”, a linear peptide containing histidine residues; and “E8” a linear peptide containing glutamate residues. Other linear and branched lipid solubilising agents are also envisaged, including a hyper-branched peptide containing glutamate residues (see, e.g., “branched E8”, below). In yet a further embodiment of the present invention, the solubilising agent includes PEG and one or more of the group consisting of R4, H4, H8 and E8 (linear or branched). R4, H4, H8 and E8 have been previously described in PCT/AU2009/000469 (WO/2010/115230) and have the following structures:

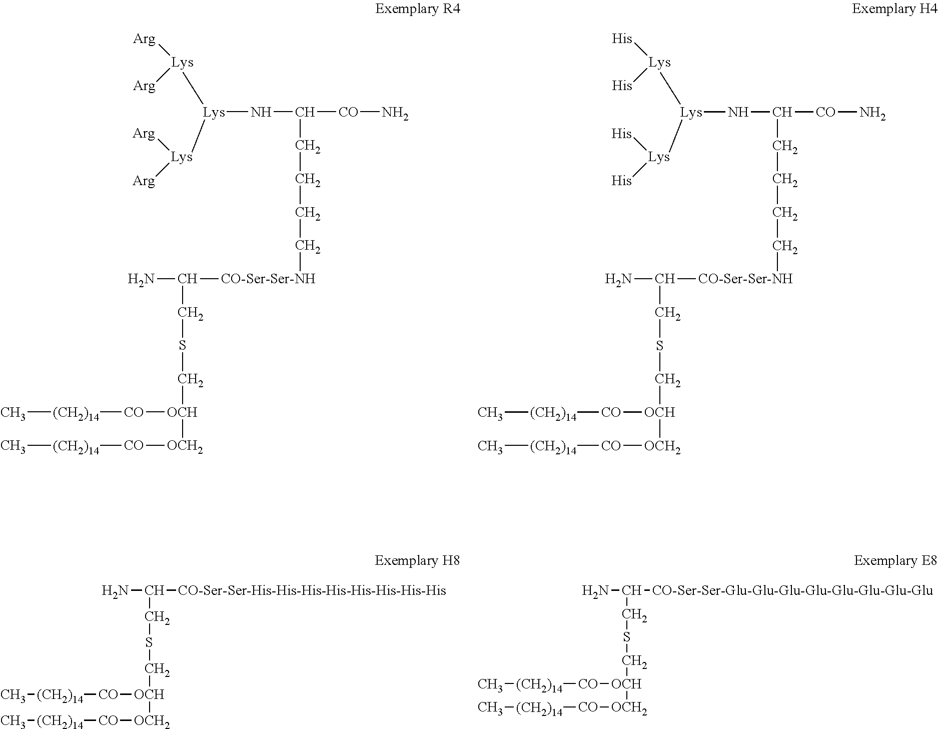

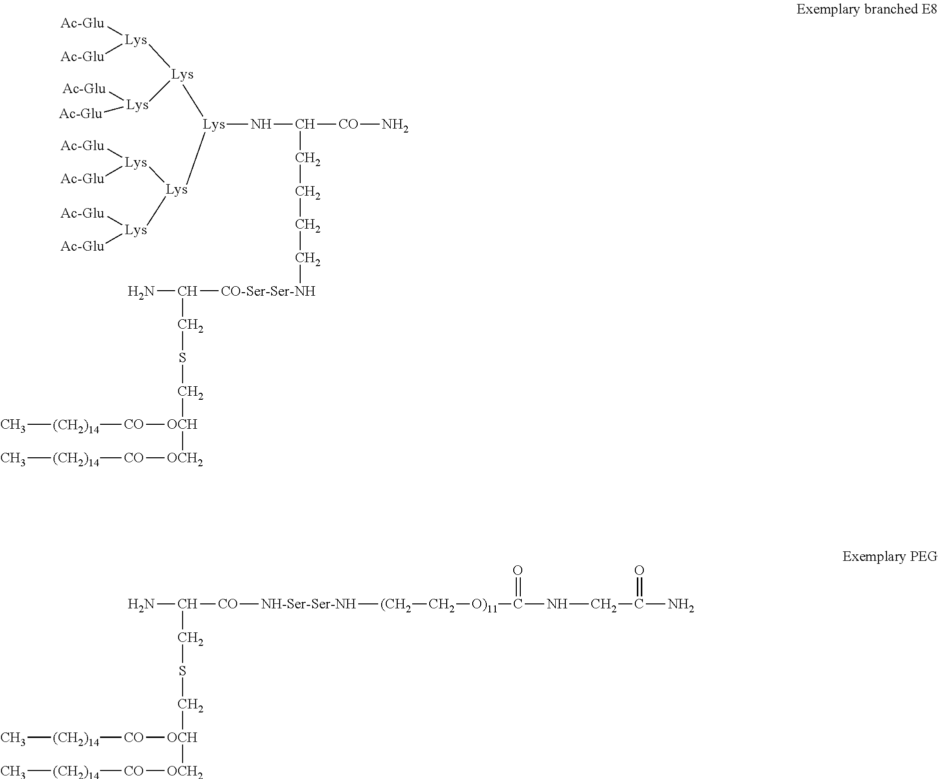

Following are schematic representations of some examples of branched (structures 1-5) and linear (structures 6-8) immunogenic compositions comprising of positively charged (Arginine, R; Lysine, K) or negatively charged (Aspartic acid, D; Glutamic acid, E) amino acids in terminal positions such that their respective electrostatic charges are displayed to the environment. Each immunogenic composition also contains dipalmitoyl-S-glyceryl cysteine (Pam2Cys) which is a ligand for Toll-Like Receptor 2. Two serine residues (Ser) are also incorporated. In the case of construct 2 the peptide structure was assembled in the direction N→C, all other structures shown in the figure were assembled C→N. Positive and negative electrostatic charges are shown as 2−, 2+, 1−, 1+ etc. depending on the size of charge. Ac=acetyl group used to suppress the positive charge of alpha amino groups in the case of N-terminally situated Glutamic acid.

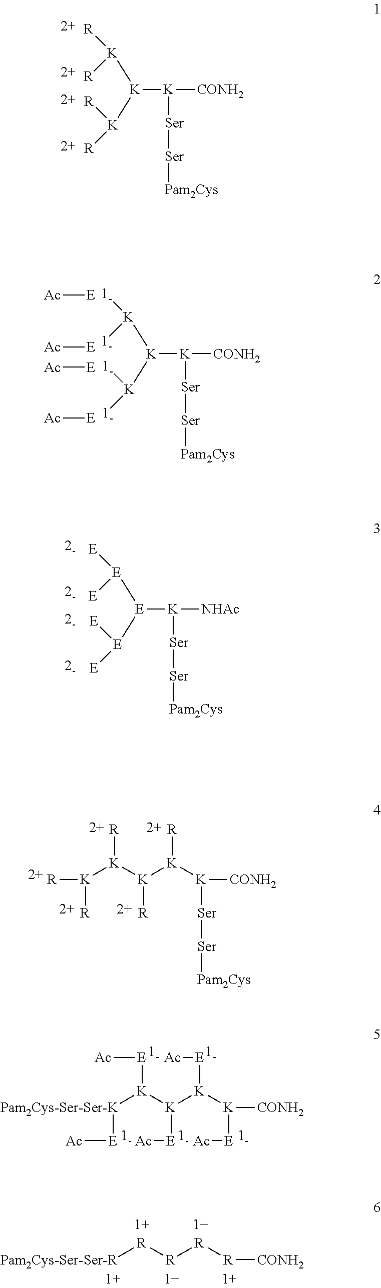

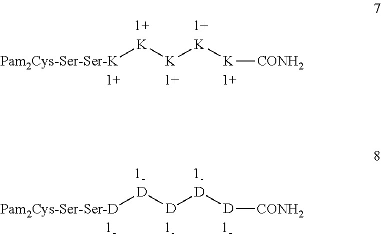

A person skilled in the art will appreciate that the present invention is not limited to the particular exemplified functional groups that can act as solubilising moieties, and that other suitable functional groups including those that can act as solubilising agents known in the art may be used in accordance with the present invention, such as carbohydrates.

The way in which the one or more solubilising moieties as described herein may be conjugated to a lipid according to the present invention would be well known to a person skilled in the art. For example, conjugation via Fmoc chemistry, through a disulfide or a thioether bridge, or via oxime chemistry is envisaged. In a particular embodiment of the present invention, a soluble form of Pam2Cys was prepared by addition of O—(N-Fmoc-2-aminoethyl)-O′-(2-carboxyethyl)-undecaethyleneglycol (Fmoc-PEOn-OH, Merck Ltd) to Pam2Cys. This resulted in the formation of a PEGylated form of the lipid, Pam2Cys-PEG₁₁.

In another form of the invention, the TLR2 agonist moiety comprises a conjugate comprising Pam2Cys conjugated to a pendant R4 form. In a preferred form, pendant-Pam2Cys is conjugated to R4 according to the following structure:

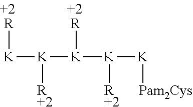

In a preferred form according to any embodiment of the present invention, the TLR2 agonist moiety comprises a conjugate comprising Pam2Cys conjugated to PEG. In a preferred form according to any embodiment of the present invention, the TLR2 agonist moiety comprises a conjugate comprising Pam2Cys conjugated to PEG11 or PEG12. Preferably, the Pam2Cys and PEG11 or PEG12 molecules are separated by at least two serines (PEG₁₁-SS-Pam2Cys or PEG12-SS-Pam2Cys).

As used herein, reference to a TLR2 agonist also includes a pharmaceutically acceptable salt, solvate, polymorph or prodrug thereof. Accordingly, any compound of the invention described herein may be included in the pharmaceutical compositions in the form of a pharmaceutically acceptable salt, solvate, polymorph or prodrug thereof.

Additional compounds that comprise a TLR2 agonist moiety that are useful in any aspect of the present invention are described below.

In any aspect, the compound may be a compound of formula (I):

A-Y-B (I)
wherein A comprises or consists of a moiety selected from A1 and A2:

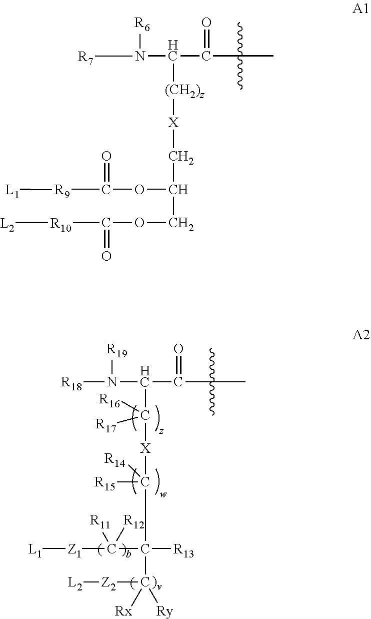

wherein
each z is independently selected from 1 or 2;
each X is independently selected from —S—, —S(═O)— and —S(═O)₂—;
R₆ and R₇ are independently selected from the group consisting of H, a straight or branched C₁-C₄ alkyl, and —C(═O)CH₃; and
b and w are each independently an integer from 0 to 7 and v is an integer from 0 to 5, such as from 2 to 5, provided that:
   the sum of b, v, and w is at least 3; and
   the sum of b and w is from 0 to 7;
Z₁ and Z₂ are each independently selected from the group consisting of —O—, —NR—, —S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A2;
L₁ and L₂ are each independently C₅-C₂₁ aliphatic or C₄-C₂₀ heteroaliphatic;
   L₃ is C₁-C₂₁ aliphatic or C₂-C₂₀ heteroaliphatic;
   A₂ is an amino acid or a peptide;
   wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
Y is

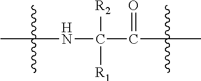

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
and
B comprises or consists of Polyethylene Glycol (PEG),
or a pharmaceutically acceptable salt, solvate or prodrug thereof.

In any aspect, the compound may be a compound of formula (IA1):

A-Y-B (IA1)
wherein A comprises or consists of moiety A1:

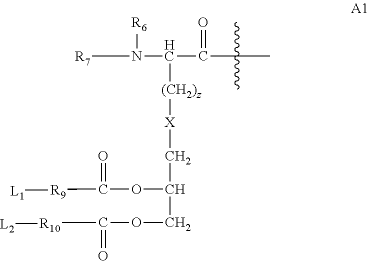

wherein:
L₁ and L₂ are each independently C₅-C₂₁ aliphatic or C₄-C₂₀ heteroaliphatic;
z is 1 or 2;
X is selected from —S—, —S(═O)— and —S(═O)₂—;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond;
Y is

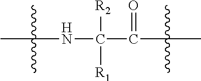

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ is selected from the group consisting of H and a straight or branched C₁-C₅ alkyl;
and
B comprises or consists of Polyethylene Glycol (PEG),
or a pharmaceutically acceptable salt, solvate or prodrug thereof.

In any aspect or embodiment, any compound comprising moiety A1 described herein may be provided as moiety AX:

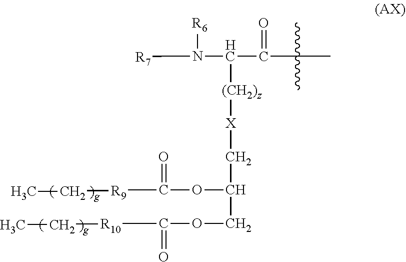

wherein:
each g is independently 10, 11, 12, 13, 14, 15, 16, 17 or 18; and
R₉ and R₁₀ are independently selected from the group consisting of —NH—, —O— or a single bond; and
R₆, R₇, X and z have the meanings given in for moiety A1.

In some embodiments, g is an integer from 12 to 16.

In some embodiments, g is 14.

In any aspect, the compound may be a compound of formula (IA2):

A-Y-B (IA2)
wherein A comprises or consists of:

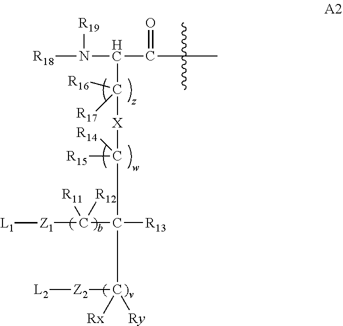

wherein
b and w are each independently an integer from 0 to 7 and v is an integer from 0 to 5, such as from 2 to 5, provided that:
   the sum of b, v, and w is at least 3; and
   the sum of b and w is from 0 to 7;
z is 1 or 2;
X is selected from —S—, —S(═O)— and —S(═O)₂—;
Z₁ and Z₂ are each independently selected from the group consisting of —O—, —NR—, —S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ are each independently H or C₁-C₆ aliphatic;
R, R₁₃ and R₁₈ are each independently H or C₁-C₈ aliphatic;
R₁₉ is H, C₁-C₆ aliphatic, an amino protecting group, L₃-C(═O)—, or A2;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
Y is

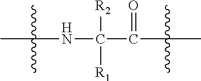

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
and
B comprises or consists of Polyethylene Glycol (PEG),
or a pharmaceutically acceptable salt, solvate or prodrug thereof.

In some embodiments, v is an integer selected from 2, 3, 4 or 5. In some embodiments, v is 2 or 3. In some embodiments, v is 2.

In some embodiments, Rₓ, R_{y}, R₁₁, R₁₂, R₁₃, R₁₄, R₁₅, R₁₆, and R₁₇ are H.

In some embodiments, R and R₁₃ are each H.

In some embodiments, Z₁ and Z₂ are the same and selected from the group consisting of —O—, —NR—, —S—, S(═O), S(═O)₂—, —C(═O)O—, —OC(═O)—, —C(═O)NR—, —NRC(═O)—, —C(═O)S—, —SC(═O)—, OC(═O)O—, NRC(═O)O—, —OC(═O)NR—, and —NRC(═O)NR—.

In some embodiments, Z₁ and Z₂ are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—.

In some embodiments, w is an integer selected from 1-7. In some embodiments, w is 1.

In some embodiments, b is 0.

In some embodiments, the sum of b and w is from 1 to 7. In these embodiments, b may be an integer selected from 0-7 and w may be an integer selected from 1-7, preferably 1.

In some embodiments, b is 0, w is 1 and v is 2.

In some embodiments, R₁₈ is H.

In some embodiments, R₁₉ is selected from the group consisting of H, C₁-C₆ alkyl, —C(═O) C₁-C₆ alkyl or —C(═O)C₁₁-C₁₉alkyl.

In some embodiments, R₁₉ is selected from H, C₁-C₆ alkyl, —C(═O) C₁-C₄ alkyl, preferably H, C₁-C₄ alkyl, —C(═O) C₁-C₄ alkyl.

In some embodiments, R₁₉ is selected from H and —C(═O)CH₃.

In some embodiments, L₁ and L₂ are independently selected from C₅-C₂₁ aliphatic or C₄-C₂₀ heteroaliphatic. In some embodiments, L₁ and L₂ and independently selected from C₁₀-C₁₈ aliphatic or C₁₀-C₁₈ heteroaliphatic. In some embodiments, L₁ and L₂ are independently selected from C₁₄-alkyl and C₁₅-alkyl.

In some embodiments, X is S.

In some embodiments, X is S(═O).

In some embodiments, X is S(═O)₂.

In some embodiments, R₆ and R₇ are each H.

In some embodiments, R₁₈ and R₁₉ are each H.

In some embodiments, the invention provides a compound of formula (I) wherein:
v is an integer from 2 to 5;
b is 0;
Rₓ, R_{y}, R₁₃, R₁₄, R₁₅, R₁₆, and R₁₇ are H;
Z₁ and Z₂ are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
w is an integer from 1 to 7;
R₁₉ is selected from the group consisting of H, C₁-C₆ alkyl, —C(═O) C₁-C₆ alkyl or —C(═O)C₁₁-C₁₉alkyl; and
L₁ and L₂ and independently selected from C₁₀-C₁₈ aliphatic or C₁₀-C₁₈ heteroaliphatic.

In some embodiments, the invention provides a compound wherein
v is 2;
b is 0;
w is 1;
the sum of v, b and w is 3;
the sum of b and w is 1;
z is 1;
X is S
Z₁ and Z₂ are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each H;
R and R₁₃ are each H;
R₁₈ is H;
R₁₉ is selected from the group consisting of H, C₁-C₆ alkyl, —C(═O) C₁-C₆ alkyl or —C(═O)C₁₁-C₁₉alkyl; and
L₁ and L₂ and independently selected from C₁₀-C₁₈ aliphatic or C₁₀-C₁₈ heteroaliphatic.

It will be appreciated that any embodiment of a substituent described herein, including substituents R, R₁, R₂, R₄, R₅, R₆, R₇, R₉, R₁₀, z, X, g, R₁₁, R₁₂, R₁₃, R₁₄, R₁₅, R₁₆, R₁₇, R₁₈, R₁₉, R²¹, R²², R²³, R²⁴, R^{24a}, R^{24b}, R²⁵, R^{25a}, R^{25b}, R²⁶, R²⁷, Rₓ, R_{y}, L₁ (or L¹), L₂ (or L²), Z₁ (or Z¹), Z₂ (or Z²), b, v, w, n, m, p, q, d, R₃, L, PEG, t, k and h, is intended to apply to any instance of that substituent for any compound described herein, including compounds of formulas (I)-(XXIII).

In any aspect, the compound may be a compound of formula (II):

A-Y′-B (II)
wherein A comprises or consists of moiety A1 or A2 as defined herein;
Y′ is

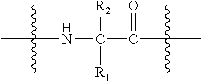

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
and
B comprises or consists of Polyethylene Glycol (PEG),
or a pharmaceutically acceptable salt, solvate or prodrug thereof.

In some embodiments, the compound comprises moiety A1, wherein:
each g is independently 10, 11, 12, 13, 14, 15, 16, 17 or 18;
z is 1;
X is S;
R₆ and R₇ are H;
R₉ and R₁₀ are both a single bond.

In some embodiments, moiety A1 is defined by moiety A1′

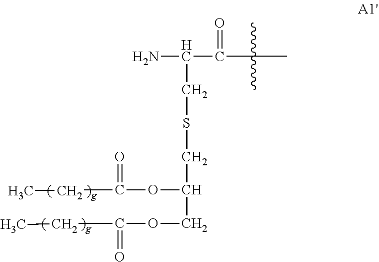

wherein each g is independently 10, 11, 12, 13, 14, 15, 16, 17 or 18.

In any aspect, any of the compounds described herein may be a compound comprising a moiety A selected from A1′ and A2 as defined herein and PEG, wherein the moiety A and PEG are linked by a glycine, serine, homoserine, threonine, phosphoserine, asparagine or glutamine residue, or an ester of a glutamine residue.

In any aspect, the compound may comprise or consist of partial structure A1Y′ or A2Y′:

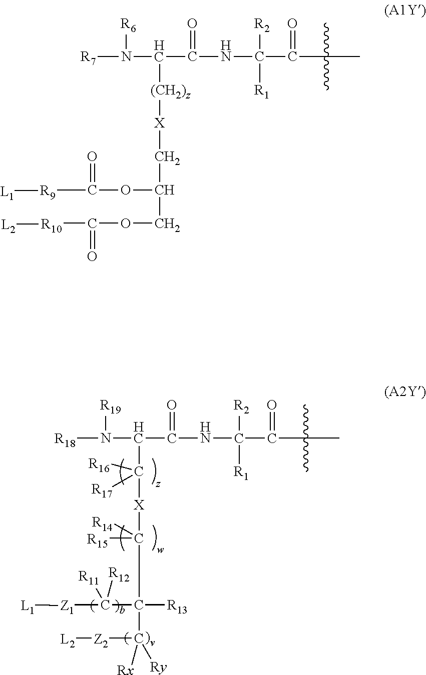

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
b and w are each independently an integer from 0 to 7 and v is an integer from 0 to 5, provided that:
the sum of b, v, and w is at least 3; and
the sum of b and w is from 0 to 7;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A2;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted; and
A1Y′ or A2Y′ is covalently linked to polyethylene glycol (PEG),
or a pharmaceutically acceptable salt, solvate or prodrug thereof.

In some embodiments, L₁ and L₂ are each independently a C₁₁-C₁₉ alkyl, preferably C₁₃-C₁₇alkyl, most preferably a C₁₅alkyl.

In some embodiments, the moiety A and PEG are linked by a serine, homoserine, threonine or phosphoserine residue.

In some embodiments, moiety A and PEG are covalently linked to the glycine, serine, homoserine, threonine, phosphoserine, asparagine or glutamine residue, or an ester of a glutamine residue, through the bond(s) denoted by [symbol] .

In any aspect, the compound may be:

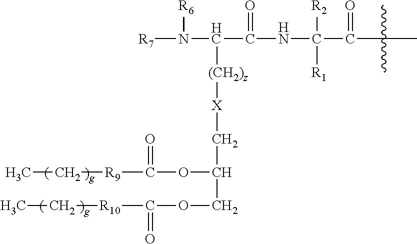

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2; and
X is selected from —S—, —S(═O)— and —S(═O)₂—;
covalently linked to polyethylene glycol (PEG),
or a pharmaceutically acceptable salt, solvate or prodrug thereof.

In some embodiments, the compound may be:

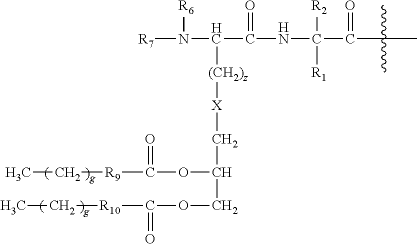

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
   R₆ and R₇ are independently selected from the group consisting of H, a straight or branched C₁-C₄ alkyl, and —C(═O)CH₃;
   R₉ and R₁₀ are independently selected from the group consisting of —NH—, —O— or a single bond;
   z is 1 or 2; and
   X is selected from —S—, —S(═O)— and —S(═O)₂—;
   covalently linked to polyethylene glycol (PEG),
   or a pharmaceutically acceptable salt, solvate or prodrug thereof.

In some embodiments, the compound may be:

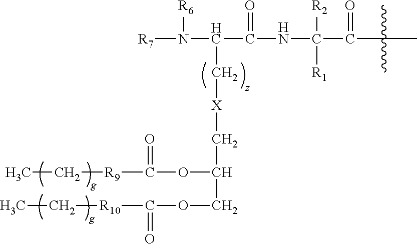

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are H;
R₈ is selected from the group consisting of H and a straight or branched C₁-C₆ alkyl;
   R₉ and R₁₀ are both a single bond;
   z is 1; and
   X is S;
   covalently linked to polyethylene glycol (PEG),
   or a pharmaceutically acceptable salt, solvate or prodrug thereof.

In some embodiments, the PEG is covalently linked through the bond denoted by [symbol] .

In some embodiments, the compound may be:

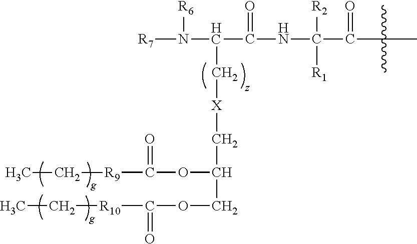

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
   R₆ and R₇ are H;
   R₉ and R₁₀ are both a single bond;
   z is 1; and
   X is S;
   covalently linked to polyethylene glycol (PEG),
   or a pharmaceutically acceptable salt, solvate or prodrug thereof.

In some embodiments, the PEG is covalently linked through the bond denoted by [symbol] .

In some embodiments, the compound may be:

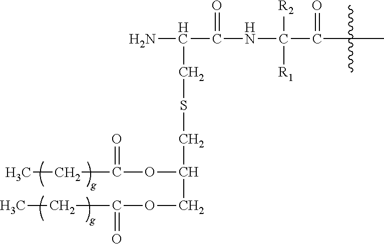

wherein R₁, R₂ and g are as defined herein. salt, solvate or prodrug thereof.

In some embodiments, the PEG is covalently linked through the bond denoted by [symbol] .

In some embodiments, the compound may be:

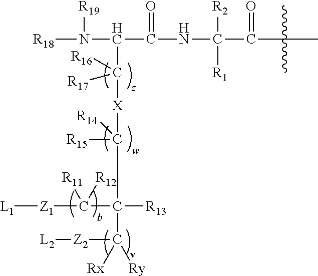

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
b and w are each independently an integer from 0 to 7 and v is an integer from 0 to 5, provided that:
the sum of b, v, and w is at least 3; and
the sum of b and w is from 0 to 7;
z is 1 or 2;
X is selected from —S—, —S(═O)— and —S(═O)₂—;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₈ aliphatic;
R₁₉ is H, C₁-C₆ aliphatic, an amino protecting group, L₃-C(═O)—, or A2;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
covalently linked to polyethylene glycol (PEG),
or a pharmaceutically acceptable salt, solvate or prodrug thereof.

In some embodiments, the PEG is covalently linked through the bond denoted by [symbol] .

In any aspect, the compound may be a compound of formula (III):

AY-B (III)
wherein
AY comprises or consists of a moiety selected from AY1 and AY2

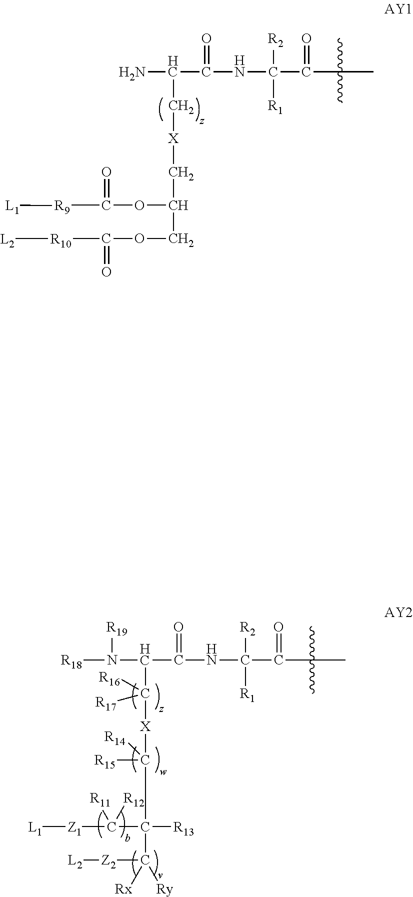

wherein each of R₁, R₂, R₆, R₇, R₉, R₁₀, z, X, R₁₁, R₁₂, R₁₃, R₁₄, R₁₅, R₁₆, R₁₇, R₁₈, R₁₉, Rₓ, R_{y}, L₁, L₂, Z₁, Z₂, b, v and w are as defined for the compound of formula (I); and
B comprises or consists of Polyethylene Glycol (PEG).

In any aspect, the compound may be a compound of formula (IV):

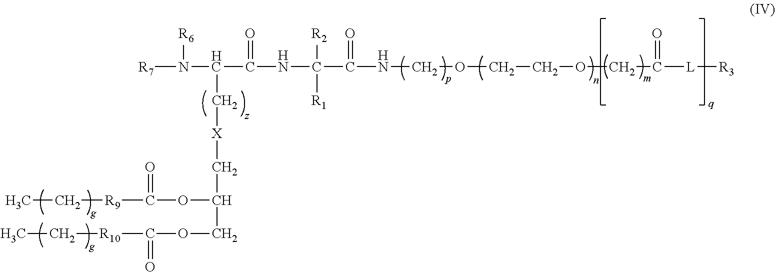

wherein
n is 3 to 100;
m is 1, 2, 3 or 4;
each g is independently 10, 11, 12, 13, 14, 15, 16, 17 or 18;
p is 2, 3 or 4;
q is null or 1;
R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

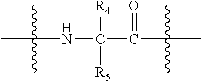

wherein R₄ is H; and
R₅ is the side chain, or second hydrogen of the amino acid
or a pharmaceutically acceptable salt, solvate or prodrug thereof.

In any aspect, the compound may be a compound of formula (V):

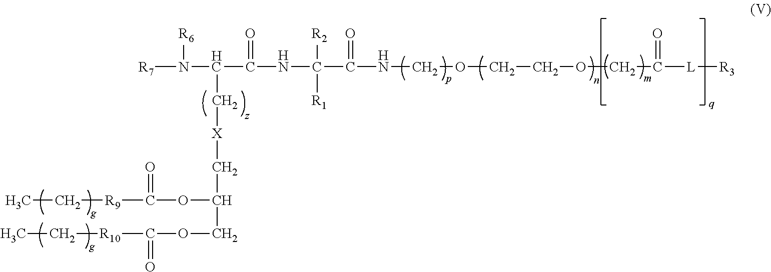

wherein
n is 3 to 100;
m is 1, 2, 3 or 4;
each g is independently 10, 11, 12, 13, 14, 15, 16, 17 or 18;
p is 2, 3 or 4;
q is null or 1;
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond;
z is 1 or 2;
X is selected from —S—, —S(═O)— and —S(═O)₂—;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

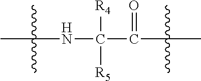

wherein R₄ is H; and
R₅ is the side chain, or second hydrogen of the amino acid
or a pharmaceutically acceptable salt, solvate or prodrug thereof.

In some embodiments, the compound is a compound of formula (IV) or (V) wherein
R₆ and R₇ are H;
R₉ is selected from the group consisting of H and a straight or branched C₁-C₆ alkyl;
R₉ and R₁₀ are both a single bond;
z is 1; and
X is S.

In some embodiments, the compound of any one of formulas (I)-(V) may be a compound of formula (VI):

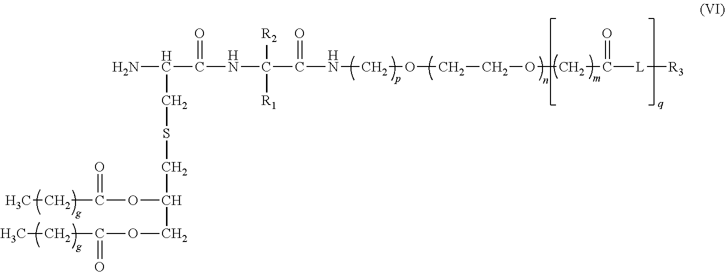

wherein
n is 3 to 100;
m is 1, 2, 3 or 4;
each g is independently 10, 11, 12, 13, 14, 15, 16, 17 or 18;
p is 2, 3 or 4;
q is null or 1;
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

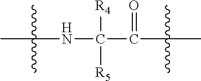

wherein R₄ is H; and
R₅ is the side chain, or second hydrogen of the amino acid
or a pharmaceutically acceptable salt, solvate or prodrug thereof.

In any aspect, the compound may be a compound of formula (VII):

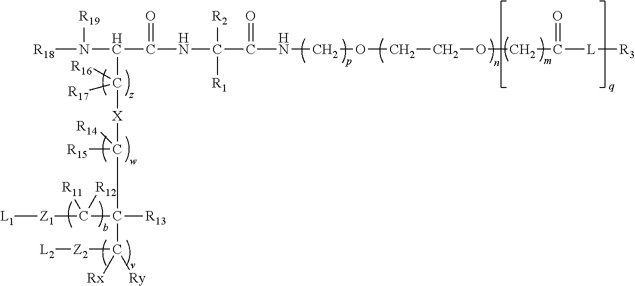

(VII)
wherein
n is 3 to 100;
m is 1, 2, 3 or 4;
p is 2, 3 or 4;
q is null or 1;
R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and CH₂CH₂C(═O)OR₈, wherein any one of the alkyl hydrogens can be replaced with a halogen;
R₈ is selected from the group consisting of H and a straight or branched C₁-C₆ alkyl;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

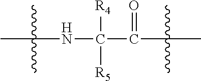

wherein R₄ is H; and
R₅ is the side chain, or second hydrogen of the amino acid;
b and w are each independently an integer from 0 to 7 and v is an integer from 0 to 5, provided that:
the sum of b, v, and w is at least 3; and
the sum of b and w is from 0 to 7;
z is 1 or 2;
X is selected from —S—, —S(═O)— and —S(═O)₂—;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A2;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
or a pharmaceutically acceptable salt, solvate or prodrug thereof.

In any aspect, the compound may be a compound of formula (VIII):

A-Y—NH—(CH₂)ₚ—O—(CH₂—CH₂—O)ₙ—[(CH₂)ₘ—CO-L-]_{q}R₃ (VIII)
wherein
A is a moiety selected from A1 and A2 as defined herein
Y is

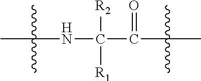

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
n is 3 to 100;
m is 1, 2, 3 or 4;
each g is independently 10, 11, 12, 13, 14, 15, 16, 17 or 18;
p is 2, 3 or 4;
q is null or 1;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

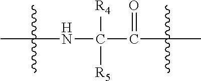

wherein R₄ is H; and
R₅ is the side chain, or second hydrogen of the amino acid,
or a pharmaceutically acceptable salt, solvate or prodrug thereof.

In any aspect, the compound may be a compound of formula (IX):

A1-Y—NH—(CH₂)ₚ—O—(CH₂—CH₂—O)ₙ—[(CH₂)ₘ—CO-L-]_{q}R₃ (IX)
wherein
A1 is represented by moiety A1 as defined for formula (I)
Y is

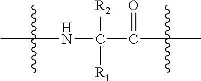

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond;
z is 1 or 2;
X is selected from —S—, —S(═O)— and —S(═O)₂—;
n is 3 to 100;
m is 1, 2, 3 or 4;
each g is independently 10, 11, 12, 13, 14, 15, 16, 17 or 18;
p is 2, 3 or 4;
q is null or 1;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

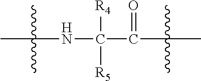

wherein R₄ is H; and
R₅ is the side chain, or second hydrogen of the amino acid,
or a pharmaceutically acceptable salt, solvate or prodrug thereof.

In some embodiments, the compound is a compound of formula (VIII) or (IX), wherein
R₆ and R₇ are H;
R₉ and R₁₀ are both a single bond;
z is 1;
X is S.

In any aspect, the compound may be a compound of formula (X):

Pam2Cys-Y—NH—(CH₂)ₚ—O—(CH₂—CH₂—O)ₙ—[(CH₂)ₘ—CO-L-]_{q}R₃ (X)
wherein
Pam2Cys has the structure:

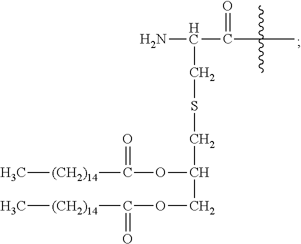

Y is:

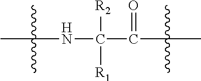

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
n is 3 to 100;
m is 1, 2, 3 or 4;
p is 2, 3 or 4;
q is null or 1;
wherein when q=1, R₃ is H, —NH₂ or —OH;
wherein when q=0, R₃ is H;
L is null or consists of 1 to 10 units, wherein each unit is a natural alpha amino acid or derived from a natural alpha amino acid, and has the formula:

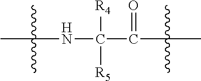

wherein R₄ is H; and
R₅ is the side chain, or second hydrogen of the amino acid,
or a pharmaceutically acceptable salt, solvate or prodrug thereof.

In any aspect, the compound may be a compound of formula (XI):

Pam2Cys-Y—NH—(CH₂)ₚ—O—(CH₂—CH₂—O)ₙ—[(CH₂)ₘ—CO-L-]_{q}R₃ (XI)
wherein
Pam2Cys has the structure:

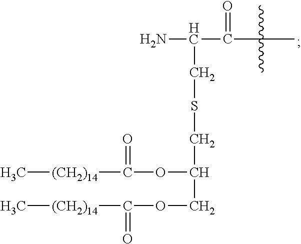

Y is:

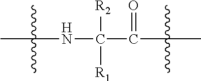

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen and wherein R₁ and R₂ are not both H;
n is 3 to 100;
m is 1, 2, 3 or 4;
p is 2, 3 or 4;
q is null or 1;
wherein when q=1, R₃ is H, —NH₂ or —OH;
wherein when q=0, R₃ is H;
L is null or consists of 1 to 10 units, wherein each unit is a natural alpha amino acid or derived from a natural alpha amino acid, and has the formula:

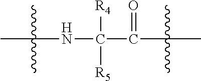

wherein R₄ is H; and
R₅ is the side chain, or second hydrogen of the amino acid,
or a pharmaceutically acceptable salt, solvate or prodrug thereof.

In any aspect, the compound may be a compound of formula (XII):

Pam2Cys-Y—NH—(CH₂)ₚ—O—(CH₂—CH₂—O)ₙ—[(CH₂)ₘ—CO-L-]_{q}R₃ (XII)
wherein
Pam2Cys has the structure:

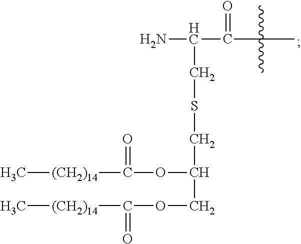

Y is:

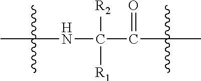

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
n is 3 to 100;
m is 1, 2, 3 or 4;
p is 2, 3 or 4;
q is null or 1;
wherein when q=1, R₃ is H, —NH₂ or —OH;
wherein when q=0, R₃ is H;
L is null or consists of 1 to 10 units, wherein each unit is a natural alpha amino acid or derived from a natural alpha amino acid, and has the formula:

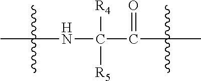

wherein R₄ is H; and
R₅ is the side chain, or second hydrogen of the amino acid,
or a pharmaceutically acceptable salt, solvate or prodrug thereof.

In any aspect, the compound may be a compound of formula (XIII):

Pam2Cys-Ser-NH—(CH₂)ₚ—O—(CH₂—CH₂—O)ₙ—[(CH₂)ₘ—CO-L-]_{q}R₃ (XIII)
wherein
Pam2Cys-Ser has the structure:

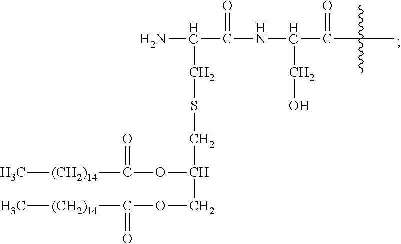

n is 3 to 100;
m is 1, 2, 3 or 4;
p is 2, 3 or 4;
q is null or 1;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

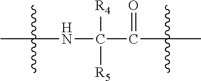

wherein R₄ is H; and
R₅ is the side chain, or second hydrogen of the amino acid,
or a pharmaceutically acceptable salt, solvate or prodrug thereof.

In one embodiment, the compound has the formula (XIV):

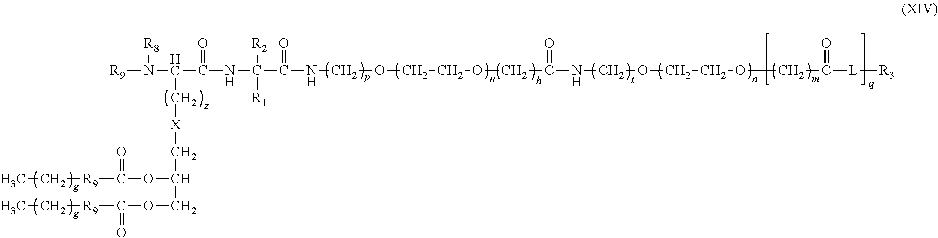

wherein
n is 3 to 100;
k is 3 to 100;
m is 1, 2, 3 or 4;
each g is independently 10, 11, 12, 13, 14, 15, 16, 17 or 18;
p is 2, 3 or 4;
t is 2, 3 or 4;
h is 1, 2, 3 or 4;
q is null or 1;
wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

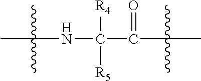

wherein R₄ is H; and
R₅ is the side chain, or second hydrogen of the amino acid,
or a pharmaceutically acceptable salt, solvate or prodrug thereof.

In one embodiment, the compound has the formula (XV):

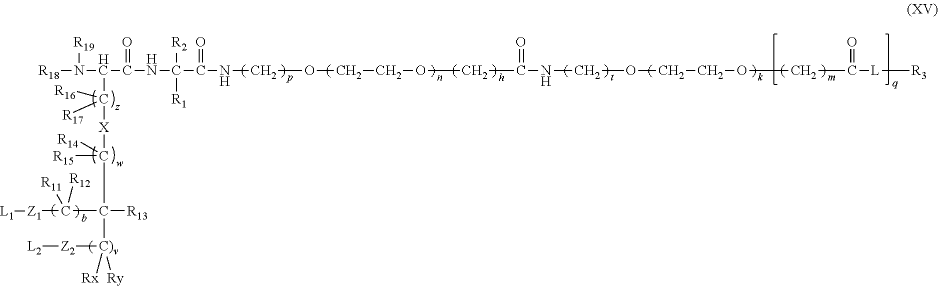

wherein
n is 3 to 100;
k is 3 to 100;
m is 1, 2, 3 or 4;
p is 2, 3 or 4;
t is 2, 3 or 4;
h is 1, 2, 3 or 4;
q is null or 1;
wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

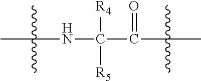

wherein R₄ is H; and
R₅ is the side chain, or second hydrogen of the amino acid;
b and w are each independently an integer from 0 to 7 and v is an integer from 0 to 5, provided that:
the sum of b, v, and w is at least 3; and
the sum of b and w is from 0 to 7;
z is 1 or 2;
X is selected from —S—, —S(═O)— and —S(═O)₂—;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A2;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₅, R₁₉, Rₓ, R_{y}, L₁, L₂, and L₃ is optionally substituted;
or a pharmaceutically acceptable salt, solvate or prodrug thereof.

In any aspect, the compound may be a compound of formula (XVI):

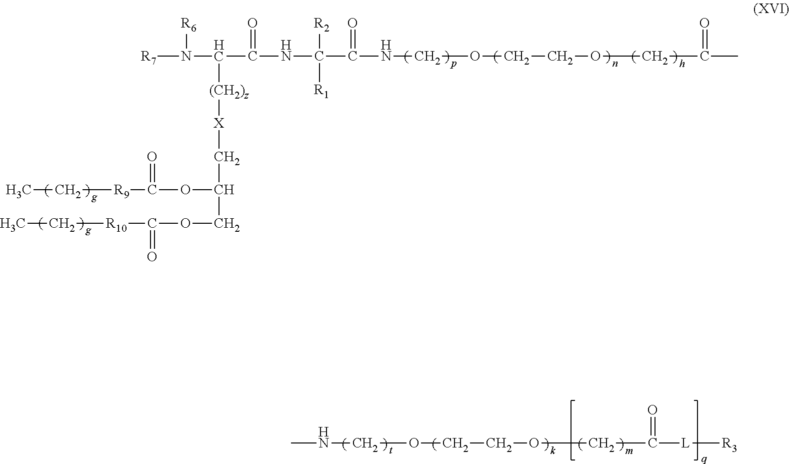

wherein
n is 3 to 100;
k is 3 to 100;
m is 1, 2, 3 or 4;
each g is independently 10, 11, 12, 13, 14, 15, 16, 17 or 18;
p is 2, 3 or 4;
t is 2, 3 or 4;
h is 1, 2, 3 or 4;
q is null or 1;
wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond;
z is 1 or 2;
X is selected from —S—, —S(═O)— and —S(═O)₂—;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

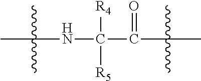

wherein R₄ is H; and
R₅ is the side chain, or second hydrogen of the amino acid,
or a pharmaceutically acceptable salt, solvate or prodrug thereof.

In any aspect, the compound may be a compound of formula (XVII):

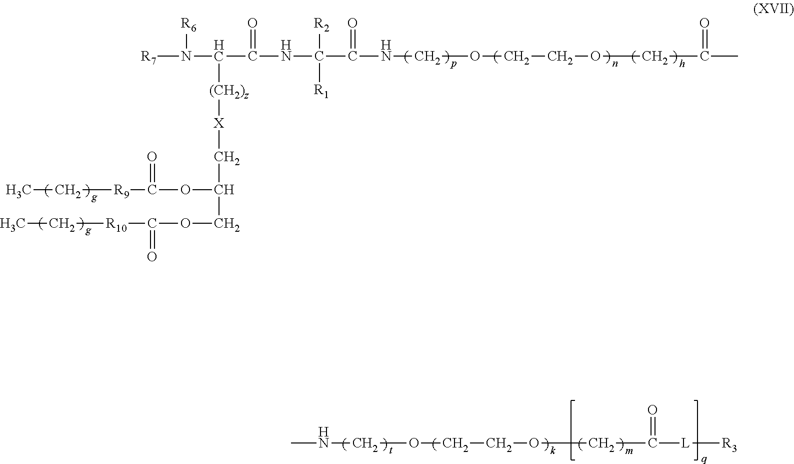

wherein
n is 3 to 100;
k is 3 to 100;
m is 1, 2, 3 or 4;
each g is independently 10, 11, 12, 13, 14, 15, 16, 17 or 18;
p is 2, 3 or 4;
t is 2, 3 or 4;
h is 1, 2, 3 or 4;
q is null or 1;
wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR, wherein any one of the alkyl hydrogens can be replaced with a halogen;
R₆ and R₇ are H;
R₆ is selected from the group consisting of H and a straight or branched C₁-C₆ alkyl;
R₉ and R₁₀ are both a single bond;
z is 1;
X is S;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

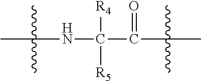

wherein R₄ is H; and
R₅ is the side chain, or second hydrogen of the amino acid,
or a pharmaceutically acceptable salt, solvate or prodrug thereof.

In any aspect, the compound may be a compound of formula (XVIII):

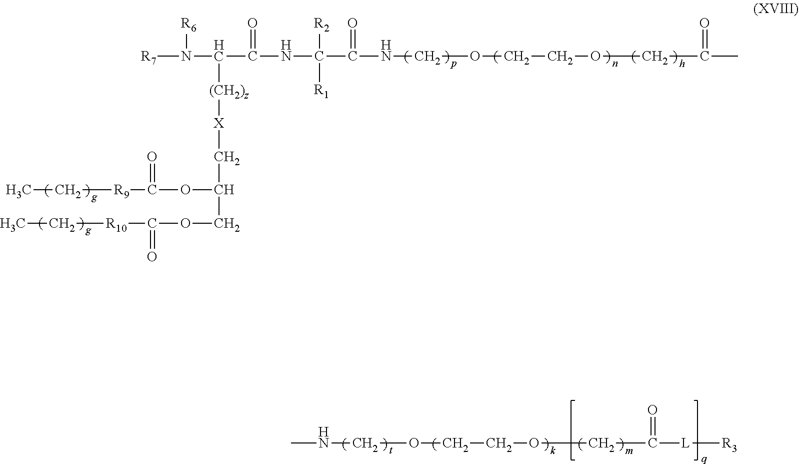

wherein
n is 3 to 100;
k is 3 to 100;
m is 1, 2, 3 or 4;
each g is independently 10, 11, 12, 13, 14, 15, 16, 17 or 18;
p is 2, 3 or 4;
t is 2, 3 or 4;
h is 1, 2, 3 or 4;
q is null or 1;
wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
R₆ and R₇ are H;
R₉ and R₁₀ are both a single bond;
z is 1;
X is S;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

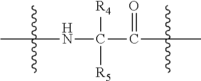

wherein R₄ is H; and
R₅ is the side chain, or second hydrogen of the amino acid,
or a pharmaceutically acceptable salt, solvate or prodrug thereof.

In any aspect, the compound may be a compound of formula (XIX):

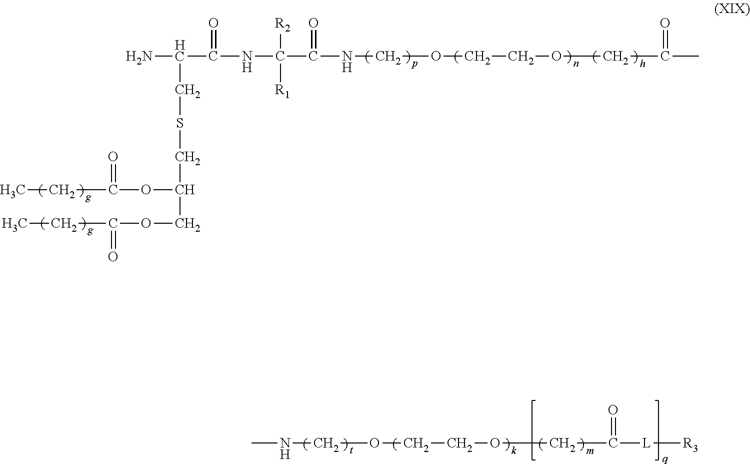

wherein
n is 3 to 100;
k is 3 to 100;
m is 1, 2, 3 or 4;
each g is independently 10, 11, 12, 13, 14, 15, 16, 17 or 18;
p is 2, 3 or 4;
t is 2, 3 or 4;
h is 1, 2, 3 or 4;
q is null or 1;
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

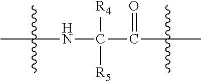

wherein R₄ is H; and
R₅ is the side chain, or second hydrogen of the amino acid,
or a pharmaceutically acceptable salt, solvate or prodrug thereof.

In one aspect, the present invention provides a compound of formula (XX):

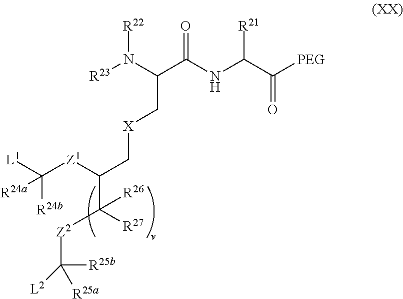

wherein:
R²¹ is selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R²² is H, C₁-C₆ aliphatic, an amino protecting group, L³-C(═O)—, or A²;
L¹ and L² are each independently C₆-C₂₁ aliphatic or C₅-C₂₀ heteroaliphatic;
L³ is C₁-C₂₁ aliphatic or C₂-C₂₀ heteroaliphatic;
A² is an amino acid or a peptide;
R²³ is H or C₁-C₆ aliphatic;
R^{24a} and R^{25a} are each independently selected from H, C₁-C₆ aliphatic and C₁-C₆ heteroaliphatic and R^{24b} and R^{25b} are each independently selected from H, C₁-C₆ aliphatic and C₁-C₆ heteroaliphatic, or R^{24a} and R^{24b} together with the carbon atom to which they are attached form a C₃₋₈cycloalkyl or 3-8 membered heterocyclyl group, and/or
R^{25a} and R^{25b} together with the carbon atom to which they are attached form a C₃₋₈cycloalkyl or 3-8 membered heterocyclyl group;
X is selected from —S—, —S(═O)— and —S(═O)₂—;
v is an integer from 1-3
R²⁶ and R²⁷ are each independently selected from H and C₁-C₆ aliphatic;
Z¹ and Z² are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—; wherein each R is independently selected from H and C₁-C₆ aliphatic;
PEG is a polyethylene glycol;
wherein any aliphatic, heteroaliphatic, cycloalkyl and heterocyclyl present in any of R²¹, R²², R²³, R^{24a}, R^{24b}, R^{25a}, R^{25b}, R²⁶, R²⁷, L¹, L² and L³ is optionally substituted.
   R^{24a}, R^{24b}, R^{25a} and R^{26b}

In some embodiments of the compound of formula (XX) comprises a branched lipid moiety. In some embodiments, These compounds of the invention may possess branching at the carbon atom bonded to L¹/L² and Z¹/Z² in formula (XX).

For example, a branched lipid compound may be a compound of formula (XX), wherein:
R^{24a} and R^{25a} are each independently selected from C₁-C₆ aliphatic and C₁-C₆ heteroaliphatic and R^{24b} and R^{25b} are each independently selected from H, C₁-C₆ aliphatic and C₁-C₆ heteroaliphatic, or R^{24a} and R^{24b} together with the carbon atom to which they are attached form a C₃₋₈cycloalkyl or 3-8 membered heterocyclyl group, and/or
R^{25a} and R^{25b} together with the carbon atom to which they are attached form a C₃₋₈cycloalkyl or 3-8 membered heterocyclyl group; Without wishing to be bound by theory, it is believed that branching at this position provides steric hindrance around the Z¹/Z² group making the compounds more resistant to degradation.

In some embodiments, R^{24a} and R^{25a} are each independently selected from C₁-C₆ aliphatic and C₁-C₆ heteroaliphatic and R^{24b} and R^{25b} are each independently selected from H, C₁-C₆ aliphatic and C₁-C₆ heteroaliphatic.

In some embodiments, R^{24a} and R^{24b} together with the carbon atom to which they are attached form a C₃₋₈cycloalkyl or 3-8 membered heterocyclyl group.

In some embodiments, R^{25a} and R^{25b} together with the carbon atom to which they are attached form a C₃₋₈cycloalkyl or 3-8 membered heterocyclyl group.

In some embodiments, R^{24a} is the same as R^{25a}.

In some embodiments, R^{24b} is the same as R^{25b}.

In some embodiments, R^{24b} and R^{25b} are H.

In some embodiments, R^{24a} and R^{25a} are each independently C₁-C₆ aliphatic. In these embodiments, R^{24a} and R^{25a} each may independently be a C₁-C₆ alkyl. In some embodiments, R^{24a} and R^{25a} are each methyl.

In some embodiments, R^{24a} and R^{24b} together with the carbon atom to which they are attached form a C₃₋₈cycloalkyl or a 3-8 membered heterocyclyl selected from: cyclohexanyl, piperidinyl, cyclopentanyl, cycloheptanyl, epoxide, cyclopropyl, cyclobutyl, oxiranyl, aziridinyl, and pyranyl.

In some embodiments, R^{25a} and R^{25b} together with the carbon atom to which they are attached form a C₃₋₈cycloalkyl or a 3-8 membered heterocyclyl selected from: cyclohexanyl, piperidinyl, cyclopentanyl, cycloheptanyl, epoxide, cyclopropyl, cyclobutyl, oxiranyl, aziridinyl, and pyranyl.

R²⁶, R²⁷ and v

In some embodiments, R²⁶ and R²⁷ are the same.

In some embodiments, R²⁶ and R²⁷ are selected from H and methyl.

In some embodiments, one of R²⁶ and R²⁷ is H.

In some embodiments, R²⁶ is H.

In some embodiments, R²⁷ is H.

In some embodiments, R²⁶ and R²⁷ are each H.

In some embodiments, v is an integer selected from 1, 2 or 3. In some embodiments, v is 1 or 2.

In some embodiments, v is 1. In some embodiments, v is 2.

### X

Typically, X may be as defined in any compound of the invention described herein. In some embodiments, X is —S—.

In some embodiments, X is —S(O)— or —S(═O)₂—. In some embodiments, X is —S(O)—. In some embodiments, X is —SO₂—.

R²² and R²³

In some embodiments, R²² is selected from H, C₁-C₆ aliphatic, L³-C(═O)—, or A².

In some embodiments, R²² is selected from H, C₁₋₆ alkyl, an amino protecting group, L³-C(═O)—, or A².

In some embodiments, R²² is an amino protecting group. Preferably the amino protecting group is suitable for solid phase peptide coupling.

In some embodiments, R²² is selected from H, C₁-C₆ aliphatic and L³-C(═O)—, preferably H.

In some embodiments, R²² is selected from H, C₁-C₆alkyl, —C(═O)C₁-C₆alkyl or —C(═O)C₁₁-C₁₉alkyl.

In some embodiments, R²³ is H or C₁₋₆alkyl, preferably R²³ is H or methyl, more preferably R²³ is H.

### R²¹

In some embodiments, R²¹ is —CH₂OH.

L¹ and L²

Typically, L¹ and L² may be as defined for any compound of the invention described herein. In some embodiments, L¹ and L² are independently selected from C₅-C₂₁ aliphatic or C₄-C₂₀ heteroaliphatic. In some embodiments, L¹ and L² and independently selected from C₁₀-C₁₈ aliphatic or C₁₀-C₁₈ heteroaliphatic. In some embodiments, L¹ and L² are independently selected from C₁₄-alkyl and C₁₅-alkyl. Typically, L¹ and L² represent straight chain aliphatic groups of any of the specified lengths described herein. Accordingly, in some embodiments, the only branching in the lipid moieties is provided at R^{24a}, R^{24b}, R^{25a} and R^{25b}.

Z¹ and Z²

Typically, Z¹ and Z² may be as defined for Z₁ and Z₂, respectively, for any compound of the invention described herein. In some embodiments, Z¹ and Z² are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—, wherein each R is independently H or C₁-C₆ aliphatic,

In some embodiments, Z¹ and Z² are the same.

In some embodiments, Z¹ and Z² are independently selected from —C(O)O— and —OC(O)—.

In some embodiments, Z¹ and Z² are each —C(O)O—. In these embodiments, the carbonyl carbon is directly bonded to the carbon atom bonded to L¹, R^{24a} and R^{24b} or L², R^{25a} and R^{25b}. In these embodiments, the compound may be of formula (XXI):

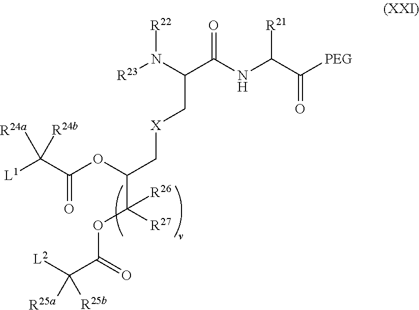

wherein each of R²¹, R²², R²³, R^{24a}, R^{24b}, R^{25a}, R^{25b}, R²⁶, R²⁷, v, X, L¹, L² and PEG have the meaning defined for any compound of the invention described herein, and wherein any aliphatic, heteroaliphatic, cycloalkyl and heterocyclyl present in any of R²¹, R²², R²³, R^{24a}, R^{24b}, R^{25a}, R^{25b}, R²⁶, R²⁷, L¹, L² and L³ is optionally substituted.

### PEG

In some embodiments, any compound disclosed herein (including a compound of any one of formulas (I)-(XXIII)) that comprises polyethylene glycol (PEG) may comprise the PEG in the form of a substituted PEG.

In the compounds of formula (XX), (XXI), (XXII) and (XXIII) PEG represents a polyethylene glycol. The polyethylene glycol includes any length polymer of ethylene oxide. The polyethylene glycol may also include substituted polyethylene glycol (“substituted PEG”). In some embodiments, substituted PEG may be defined by formulas B-I or B-II as described herein.

In some embodiments, PEG is a substituted polyethylene glycol according to the following formula B-I:

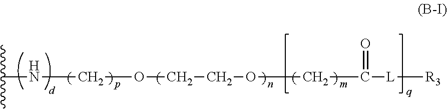

wherein
n is 3 to 100;
m is 1, 2, 3 or 4;
p is 2, 3 or 4;
q is null or 1;
d is null or 1; preferably when moiety B-I is bonded to the carbon of a carbonyl group d is 1, and when moiety B-I is bonded to the nitrogen of an amide group d is null;
R₃ is H, —NH₂ or —OH, wherein when q is null, R₃ is H and when q is 1, R₃ is —NH₂ or —OH;
L is null or consists of 1 to 10 units, wherein each unit is a natural alpha amino acid or derived from a natural alpha amino acid, and has the formula:

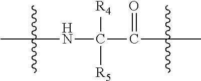

wherein R₄ is H; and
R₅ is the side chain, or second hydrogen of the amino acid.

In some embodiments, PEG is a substituted polyethylene glycol according to the following formula B-II:

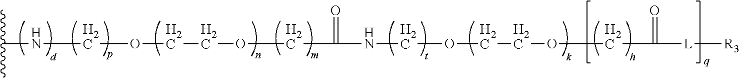

wherein
p is 2, 3 or 4;
n is 3 to 100;
m is 1, 2, 3 or 4;
t is 2, 3 or 4;
k is 3 to 100;
h is 1, 2, 3 or 4;
d is null or 1; preferably when moiety B-II is bonded to the carbon of a carbonyl group d is 1, and when moiety B-II is bonded to the nitrogen of an amide group d is null;
q is null or 1;
wherein when q is 1, R₃ is —NH₂ or —OH;
wherein when q is null, R₃ is H;
L is null or consists of 1 to 10 units, wherein each unit is a natural alpha amino acid or derived from a natural alpha amino acid, and has the formula:

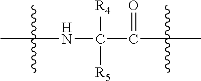

wherein R₄ is H; and
R₅ is the side chain, or second hydrogen of the amino acid.

In some embodiments of the substituted PEG of formula B-I or B-II, q is 1.

In the substituted PEG or formula B-I or B-II, n is an integer from 3 to 100. In some embodiments, n may be any sub-range within 3 to 100. In some embodiments, n has a minimum value of 4, 5, 6, 7, 8, 9, 10, 11, 15, 20, 24, 25, 26 or 27. In some embodiments, n has a maximum value of 100, 99, 98, 95, 90, 80, 70, 60, 50, 40, 30, 29, 28, 27, 25, 20, 15, 14, 13, 12 or 11. In some embodiments, n may be from any of these minimum values to any of these maximum values, for example from 4 to 100, 5 to 100, 10 to 100 or 10 to 30.

In some embodiments of the substituted PEG of formula B-I or B-II, n may be from 10 to 14, such as 11, or from 24 to 30, such as 27.

In some embodiments of the substituted PEG of formula B-I or B-II, m is from 1 to 3, such as 2.

In some embodiments of the substituted PEG of formula B-I or B-II, q is 1.

In some embodiments of the substituted PEG of formula B-I or B-II, when q is 1, R₃ is —NH₂.

In some embodiments of the substituted PEG of formula B-I or B-II, L is a natural alpha amino acid residue.

### Further Embodiments

In some embodiments, the invention provides a compound of formula (XX) wherein:
X is S;
v is an integer selected from 1 or 2;
R²⁶ and R²⁷ are H;
Z¹ and Z² are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—; each R is independently H or a C₁-C₆aliphatic;
R²² is selected from the group consisting of H, C₁-C₆ alkyl, —C(═O) C₁-C₆ alkyl or —C(═O)C₁₁-C₁₉alkyl; and
L¹ and L² and independently selected from C₁₀-C₁₃aliphatic or C₁₀-C₁₆ heteroaliphatic.

In some embodiments, the invention provides a compound of formula (XX) wherein:
R^{24b} and R^{25b} are H;
R^{24a} and R^{25a} are selected from H, C₁₋₆alkyl and C₁₋₆heteroalkyl;
X is S;
v is an integer selected from 1 or 2;
R²⁶ and R²⁷ are H;
Z¹ and Z² are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—; each R is independently H or a C₁-C₆aliphatic;
R²² is selected from the group consisting of H, C₁-C₆ alkyl, —C(═O) C₁-C₆ alkyl or —C(═O)C₁₁-C₁₉alkyl; and
L¹ and L² and independently selected from C₁₀-C₁₈aliphatic or C₁₀-C₁₆ heteroaliphatic.

In some embodiments, the invention provides a compound of formula (XX) wherein:
R^{24b} and R^{25b} are H;
R^{24a} and R^{25a} are selected from C₁₋₆alkyl and C₁₋₆heteroalkyl;
X is S;
v is an integer selected from 1 or 2;
R²⁶ and R²⁷ are H;
Z¹ and Z² are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—; each R is independently H or a C₁-C₆aliphatic;
R²² is selected from the group consisting of H, C₁-C₆ alkyl, —C(═O) C₁-C₆ alkyl or —C(═O)C₁₁-C₁₉alkyl; and
L¹ and L² and independently selected from C₁₀-C₁₈ aliphatic or C₁₀-C₁₈ heteroaliphatic.

In some embodiments, the invention provides a compound of formula (XX) wherein:
R^{24b} and R^{25b} are H;
R^{24a} and R^{25a} are selected from H and C₁₋₆alkyl;
X is S;
v is an integer selected from 1 or 2;
R²⁶ and R²⁷ are H;
Z¹ and Z² are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—; each R is independently H or a C₁-C₆aliphatic;
R²² is selected from the group consisting of H, C₁-C₆ alkyl, —C(═O) C₁-C₆ alkyl or —C(═O)C₁₁-C₁₉alkyl; and
L¹ and L² and independently selected from C₁₀-C₁₈ aliphatic or C₁₀-C₁₈ heteroaliphatic.

In some embodiments, the invention provides a compound of formula (XX) wherein:
R^{24b} and R^{25b} are H;
R^{24a} and R^{25a} are selected from C₁₋₆alkyl;
X is S;
v is an integer selected from 1 or 2;
R²⁶ and R²⁷ are H;
Z¹ and Z² are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—; each R is independently H or a C₁-C₆aliphatic;
R²² is selected from the group consisting of H, C₁-C₆ alkyl, —C(═O) C₁-C₆ alkyl or —C(═O)C₁₁-C₁₉alkyl; and
L¹ and L² and independently selected from C₁₀-C₁₆ aliphatic or C₁₀-C₁₈ heteroaliphatic.

In some embodiments, the invention provides a compound of formula (XX) wherein:
R^{24b} and R^{25b} are H;
R^{24a} and R^{25a} are selected from H and C₁₋₆alkyl;
X is S;
v is an integer selected from 1 or 2;
R²⁶ and R²⁷ are H;
Z¹ and Z² are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—; each R is independently H or a C₁-C₆aliphatic;
R²² is selected from the group consisting of H, C₁-C₆ alkyl, —C(═O) C₁-C₆ alkyl or —C(═O)C₁₁-C₁₉alkyl; and
L¹ and L² and independently selected from C₁₀-C₁₈ aliphatic.

In some embodiments, the invention provides a compound of formula (XX) wherein:
R^{24b} and R^{25b} are H;
R^{24a} and R^{25a} are selected from H and methyl;
X is S;
v is an integer selected from 1 or 2;
R²⁶ and R²⁷ are H;
Z¹ and Z² are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—; each R is independently H or a C₁-C₆aliphatic;
R²² is selected from the group consisting of H, C₁-C₆ alkyl, —C(═O) C₁-C₆ alkyl or —C(═O)C₁₁-C₁₉alkyl; and
L¹ and L² and independently selected from C₁₀-C₁₈ aliphatic or C₁₀-C₁₆ heteroaliphatic.

In some embodiments, the invention provides a compound of formula (XX) wherein:
R^{24b} and R^{25b} are H;
R^{24a} and R^{25a} are each methyl;
X is S;
v is an integer selected from 1 or 2;
R²⁶ and R²⁷ are H;
Z¹ and Z² are independently selected from the group consisting of —C(═O)O—, —OC(═O)— 20, —C(═O)NR—, —NRC(═O)—, —C(═O)S—, —SC(═O)—, —OC(═O)O—, —NRC(═O)O—, —OC(═O)NR—, and —NRC(═O)NR—; each R is independently H or a C₁-C₆aliphatic;
R²² is selected from the group consisting of H, C₁-C₆ alkyl, —C(═O) C₁-C₆ alkyl or —C(═O)C₁₁-C₁₉alkyl; and
L¹ and L² and independently selected from C₁₀-C₁₈ aliphatic or C₁₀-C₁₈ heteroaliphatic.

In some embodiments, the invention provides a compound of formula (XX) wherein:
R^{24b} and R^{25b} are H;
R^{24a} and R^{25a} are selected from H and methyl;
X is S;
v is an integer selected from 1 or 2;
R²⁶ and R²⁷ are H;
Z¹ and Z² are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—; each R is independently H or a C₁-C₆aliphatic;
R²² is selected from the group consisting of H, C₁-C₆ alkyl, —C(═O) C₁-C₆ alkyl or —C(═O)C₁₁-C₁₉alkyl; and
L¹ and L² and independently selected from C₁₀-C₁₈alkyl.

In some embodiments, the invention provides a compound of formula (XX) wherein:
R^{24b} and R^{25b} are H;
R^{24a} and R^{25a} are each methyl;
X is S;
v is an integer selected from 1 or 2;
R²⁶ and R²⁷ are H;
Z¹ and Z² are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—; each R is independently H or a C₁-C₆aliphatic;
R²² is selected from the group consisting of H, C₁-C₆ alkyl, —C(═O) C₁-C₆ alkyl or —C(═O)C₁₁-C₁₉alkyl; and
L¹ and L² and independently selected from C₁₀-C₁₈ alkyl.

In some embodiments, the invention provides a compound of formula (XX) wherein:
R^{24b} and R^{25b} are H;
R^{24a} and R^{25a} are H;
X is S;
v is an integer selected from 1 or 2;
R²⁶ and R²⁷ are H;
Z¹ and Z² are independently selected from the group consisting of —C(═O)O—;
R²² and R²³ are each H; and
L¹ and L² and independently selected from C₁₀-C₁₈ alkyl.

In some embodiments, the invention provides a compound of formula (XX) wherein:
R^{24b} and R^{25b} are H;
R^{24a} and R^{25a} are each methyl;
X is S;
v is an integer selected from 1 or 2;
R²⁶ and R²⁷ are H;
Z¹ and Z² are independently selected from the group consisting of —C(═O)O—;
R²² and R²³ are each H; and
L¹ and L² and independently selected from C₁₀-C₁₁ alkyl.

In some embodiments, the invention provides a compound of formula (XX) wherein:
R^{24b} and R^{25b} are H;
R^{24a} and R^{25a} are each H;
X is S;
v is an integer selected from 1 or 2;
R²⁶ and R²⁷ are H;
Z¹ and Z² are independently selected from the group consisting of —C(═O)O—;
R²¹ is —CH₂OH;
R²² and R²³ are each H; and
L¹ and L² and independently selected from C₁₀-C₁₈ alkyl.

In some embodiments, the invention provides a compound of formula (XX) wherein:
R^{24b} and R^{25b} are H;
R^{24a} and R^{25a} are each methyl;
X is S;
v is an integer selected from 1 or 2;
R²⁶ and R²⁷ are H;
Z¹ and Z² are independently selected from the group consisting of —C(═O)O—;
R²¹ is —CH₂OH;
R²² and R²³ are each H; and
L¹ and L² and independently selected from C₁₀-C₁₆alkyl.

In some embodiments, R^{24b} and R^{25b} are each H. In these embodiments, the compound of formula (XX) may be a compound of formula (XXII):

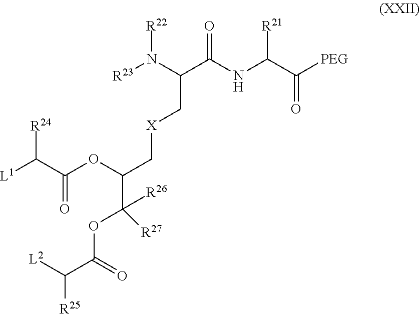

wherein each of R²¹, R²², R²³, R²⁶, R²⁷, v, X, L¹, L² and PEG have the meanings provided in any embodiment described herein and
R²⁴ and R²⁵ are independently selected from C₁-C₆ aliphatic and C₁-C₆ heteroaliphatic, wherein any aliphatic, heteroaliphatic, cycloalkyl and heterocyclyl present in any of R²¹, R²², R²³, R²⁴, R²⁵, R²⁶, R²⁷, L¹, L² and L³ is optionally substituted.
R²⁴ and R²⁵ may be any group described herein for R^{24a} and R^{25a}, respectively.

In some embodiments, R²⁴ and R²⁵ are the same.

In some embodiments, R²⁴ and R²⁵ are independently selected from C₁-C₆ alkyl and C₁-C₆ heteroaliphatic.

In some embodiments, R²⁴ and R²⁵ are independently C₁₋₆alkyl.

In some embodiments, R²⁴ and R²⁵ are independently C₁₋₄alkyl.

In some embodiments, R²⁴ and R²⁵ are each methyl.

### Alternative Forms of the Compounds

It will be understood that compounds of the invention may possess a chiral centre and may therefore exist in an R- or S-configuration. The compounds may be provided in the form of a racemate or in an enatio- or diastereo-enriched form. Enantio- and diastereo-enriched forms of the compounds may be obtained either through asymmetric synthesis, the incorporation of chiral pool materials or through a stereoselective resolution. The compounds may therefore be provided as a purified enantiomer or diastereomer, or as a mixture of any ratio thereof. The isomers may be separated conventionally by chromatographic methods or using a resolving agent. Alternatively the individual isomers may be prepared by asymmetric synthesis using chiral intermediates. Where the compound has a carbon-carbon double bond, it may occur in Z- or E-form and all isomeric forms of the compounds being included in the present invention.

Compounds described herein may exist in and be isolated in optically active and racemic forms. As would be understood by a person skilled in the art, the present invention is intended to encompass any racemic, optically active or stereoisomeric form, or mixtures thereof, of compounds of the invention which possess the useful properties described herein. It is well known in the art how to prepare such forms (for example, by resolution of racemic mixtures by recrystallization, by synthesis from optically-active starting materials, by chiral synthesis, or by chiral chromatographic separation). In some embodiments, a composition may comprise a compound in an enantiomerically or diastereomerically enriched form. For example, the compound may have an enantiomeric excess (ee) or a diastereomeric excess (de) of at least about 1%, 5%, 10%, 20%, 30%, 40%, 50%, 60%, 70%, 80%, 90%, 95%, 96%, 97%, 98%, 99% or more than 99%. In some embodiments, the compound may be enriched by at least about 1%, 5%, 10%, 20%, 30%, 40%, 50%, 60%, 70%, 80%, 90%, 95%, 96%, 97%, 98%, 99% or more than 99% at any stereocentre of the compound.

In any aspect, the compound may comprise a chiral centre around the following chiral centre (shown at *):

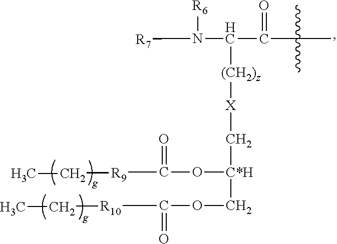

wherein the chiral centre is in the R configuration. A compound in this form may also be referred to as an R-Pam2 analogue diastereomer of a compound of the invention as described herein. This may be depicted as:

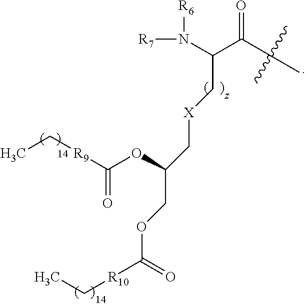

In any aspect, the compound may comprise a chiral centre in the 2,3-bis(palmitoyloxy)propyl moiety of Pam2Cys (shown at *):

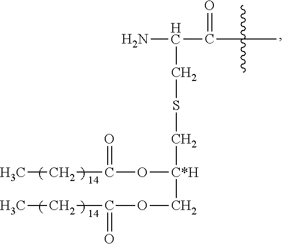

wherein the chiral centre is in the R configuration. A compound in this form may also be referred to as an R-Pam2 diastereomer of a compound of the invention as described herein. This may be depicted as:

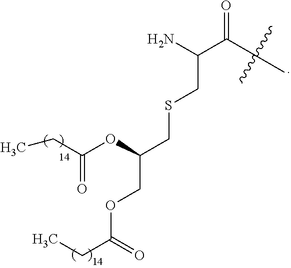

In any aspect, the compound may comprise a chiral centre around the following chiral centre (shown at *):

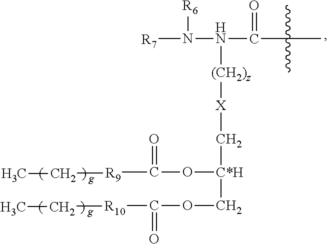

wherein the chiral centre is in the S configuration. A compound in this form may also be referred to as an S-Pam2 analogue diastereomer of a compound of the invention as described herein. This may be depicted as:

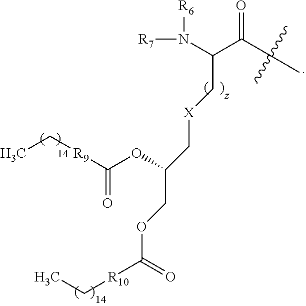

In any aspect, the compound comprises a chiral centre in the 2,3-bis(palmitoyloxy)propyl moiety of Pam2Cys (shown at *):

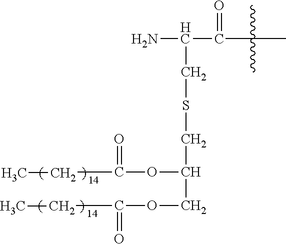

wherein the chiral centre is in the S configuration. A compound in this form may also be referred to as an S-Pam2 diastereomer of a compound of the invention as described herein. This may be depicted as:

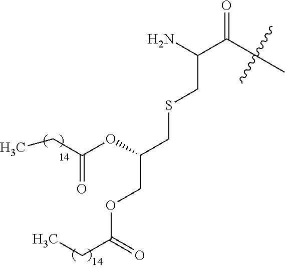

In any aspect, the compound comprises a chiral centre around the following chiral centre (shown at *):

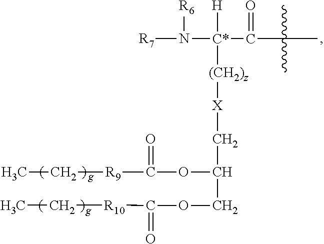

wherein the chiral centre is in the L configuration. A compound in this form may also be referred to as an L-Cys analogue diastereomer of Pam2Cys of a compound as described herein. This may be depicted as:

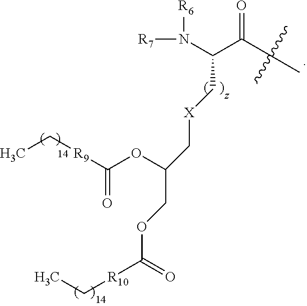

In any aspect, the compound comprises a chiral centre in the cysteine residue of Pam2Cys (shown at*)

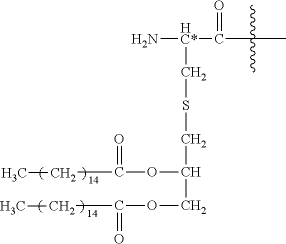

wherein the chiral centre is in the L configuration. A compound in this form may also be referred to as an L-Cys diastereomer of Pam2Cys of a compound as described herein. This may be depicted as:

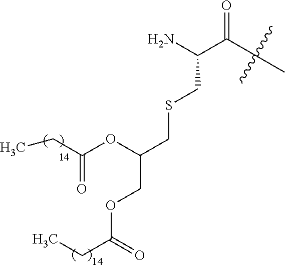

Other stereocentres in these compounds may be racemic or independently enriched in either the R or S configuration.

In any aspect, the compound comprises a chiral centre in moiety A1 around the following chiral centre (shown at *):

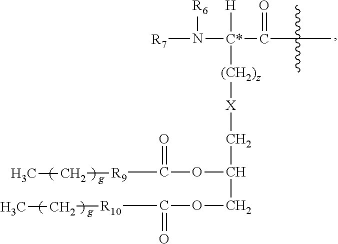

wherein the chiral centre is in the D configuration. A compound in this form may also be referred to as an D-Cys analogue diastereomer of Pam2Cys of a compound described herein. This may be depicted as:

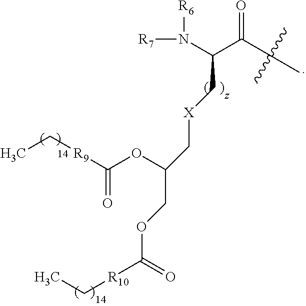

Other stereocentres in these compounds may be racemic or independently enriched in either the R or S configuration.

In any aspect, the compound comprises a chiral centre in the cysteine residue of Pam2Cys (shown at *):

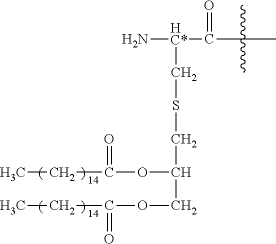

wherein the chiral centre is in the D configuration. A compound in this form may also be referred to as an D-Cys diastereomer of Pam2Cys of a compound described herein. This may be depicted as:

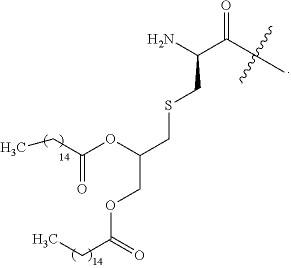

Other stereocentres in these compounds may be racemic or independently enriched in either the R or S configuration.

In any aspect or embodiment of the invention, a compound of the present invention may be provided in a chiral form enriched at a chiral centre at the following carbon atom (shown at *) of moiety A2:

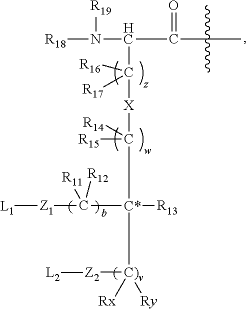

wherein the chiral centre is in the R configuration. In some embodiments, this stereoisomer of the compound may be depicted as:

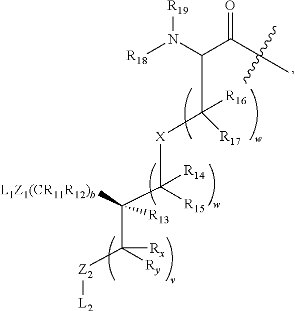

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v and z are as defined for the compound of Formula (I) and w is 1. Other stereocentres in these compounds may be racemic or independently enriched in either the R or S configuration.

In any aspect or embodiment of the invention, a compound of the present invention may be provided in a chiral form enriched at a chiral centre at the following carbon atom (shown at *) of moiety A2:

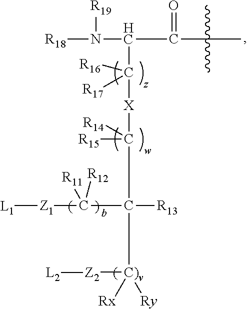

wherein the chiral centre is in the S configuration. In some embodiments, moiety A of this stereoisomer of the compound may be depicted as:

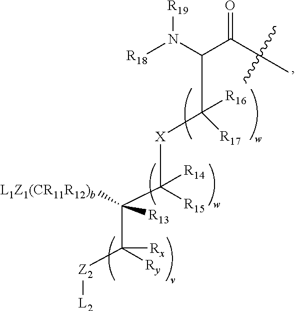

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v, w, and z are as defined for the compound or Formula (I). Other stereocentres in these compounds may be racemic or independently enriched in either the R or S configuration.

In any aspect or embodiment of the invention, a compound of the present invention may be provided in a chiral form enriched at a chiral centre at the following carbon atom (shown at **) of moiety A2:

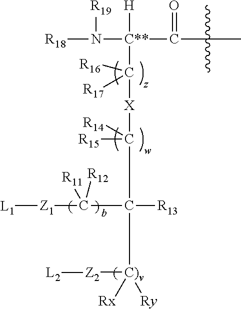

wherein the chiral centre is in the L configuration. A compound in this form may also be referred to as an L-Cys analogue stereoisomer of a compound of the invention. In some embodiments, this stereoisomer of the compound may be depicted as:

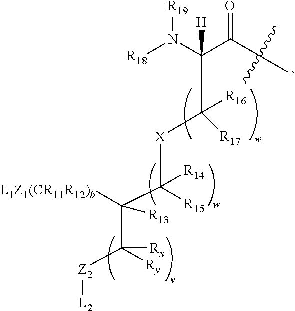

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v, w, and z are as defined for the compound or Formula (I). Other stereocentres in these compounds may be racemic or independently enriched in either the R or S configuration.

In any aspect or embodiment of the invention, a compound of the present invention may be provided in a chiral form enriched at a chiral centre at the following carbon atom (shown at **) of moiety A2:

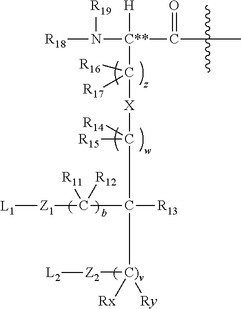

wherein the chiral centre is in the D configuration. A compound in this form may also be referred to as a D-Cys analogue stereoisomer of a compound of the invention. In some embodiments, moiety A of this stereoisomer of the compound may be depicted as:

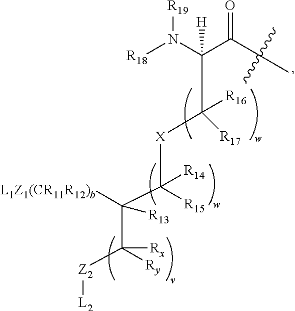

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v and z are as defined for the compound or Formula (I) and w is 1. Other stereocentres in these compounds may be racemic or independently enriched in either the R or S configuration.

The compounds of formula (XX) contain at least 6 potential stereogenic centres depending on the selection of substituents. These centres are designated with an * in formula (XX*) below.

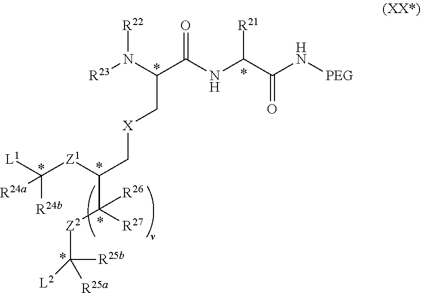

wherein R²², R²³, v, X, Z¹, Z², L¹ and L¹ have the meanings indicated above, and wherein R^{24a} is different to R^{24b}, R^{25a} is different to R^{25b}, R²¹ is not H, and R²⁶ is different to R²⁷. Any of these stereocentres may be in the R or S configuration.

In some embodiments, R²⁶ and R²⁷ are the same (e.g. each being H). In these embodiments, the remaining potential stereocentres are denoted with an * in the following formula (XX**):

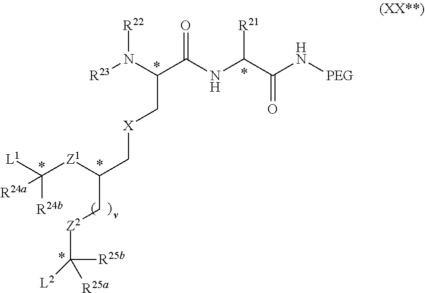

In some embodiments, the compound of formula (XX) is chiral with the conformations shown in formula (XXIII):

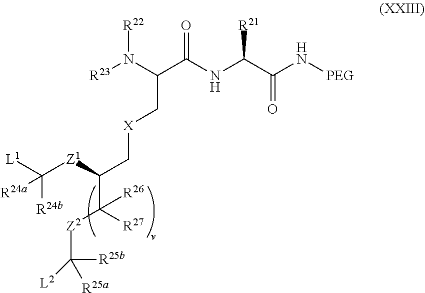

wherein each of R²¹, R²², R²³, R^{24a}, R^{24b}, R^{25a}, R^{25b}, X, Z¹, Z², v, L¹, L² and PEG have the meanings provided in any compound of the invention, and wherein any aliphatic, heteroaliphatic, cycloalkyl and heterocyclyl present in any of R²¹, R²², R²³, R^{24a}, R^{24b}, R^{25a}, R^{25b}, R²⁶, R²⁷, L¹, L² and L³ is optionally substituted.

In any aspect, the compound comprises a chiral centre in the Y moiety of the compound (shown at *):

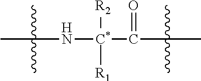

wherein the chiral centre is in the L-configuration. A compound in this form may also be referred to as an L-Y diastereomer of a compound of the invention described herein.

In any aspect, the compound comprises a chiral centre in the Y moiety of the compound (shown at *):

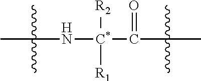

wherein the chiral centre is in the D-configuration. A compound in this form may also be

In any aspect, compositions comprising a compound of the invention (including a compound of any one of formulas (I)-(XXIII)) or a pharmaceutically acceptable salt, solvate or prodrug thereof, and a pharmaceutically acceptable carrier, diluent or excipient may be used in a method or use of the invention.

In some embodiments, the compound as described herein is the R diastereomer around the chiral centre of the 2,3-bis(palmitoyloxy)propyl moiety of the compound.

In some embodiments, the compound as described herein is the S diastereomer around the chiral centre of the 2,3-bis(palmitoyloxy)propyl moiety of the compound.

In any aspect, a composition as described herein comprises a compound that is the R diastereomer around the chiral centre of the 2,3-bis(palmitoyloxy)propyl moiety of the compound.

In any aspect, a composition comprises a compound that is the S diastereomer around the chiral centre of the 2,3-bis(palmitoyloxy)propyl moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in a composition is the R diastereomer around the chiral centre of the 2,3-bis(palmitoyloxy)propyl moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in a composition is the S diastereomer around the chiral centre of the 2,3-bis(palmitoyloxy)propyl moiety of the compound (for example moiety A1).

In any aspect, the compound as described herein is the L diastereomer around the chiral centre of the cysteine analogue residue of the Pam2Cys analogue moiety compound (for example moiety Y).

In any aspect, the compound as described herein is the L diastereomer around the chiral centre of the cysteine residue of the Pam2Cys moiety compound (for example moiety Y).

In any aspect, the compound as described herein is the D diastereomer around the chiral centre of the cysteine analogue residue of the Pam2Cys analogue moiety compound (for example moiety Y).

In any aspect, the compound as described herein is the D diastereomer around the chiral centre of the cysteine residue of the Pam2Cys moiety of the compound (for example moiety Y).

In any aspect, a composition as described herein comprises a compound that is the L diastereomer around the chiral centre of the cysteine analogue residue of the Pam2Cys analogue moiety of the compound (for example moiety Y).

In any aspect, a composition as described herein comprises a compound that is the L diastereomer around the chiral centre of the cysteine residue of the Pam2Cys moiety of the compound (for example moiety Y).

In any aspect, a composition as described herein comprises a compound that is the D diastereomer around the chiral centre of the cysteine analogue residue of the Pam2Cys analogue moiety of the compound (for example moiety Y).

In any aspect, a composition as described herein comprises a compound that is the D diastereomer around the chiral centre of the cysteine residue of the Pam2Cys moiety of the compound (for example moiety Y).

In any aspect, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the cysteine analogue residue of the Pam2Cys analogue moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the cysteine residue of the Pam2Cys moiety of the compound.

In any aspect 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the cysteine analogue residue of the Pam2Cys analogue moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the cysteine residue of the Pam2Cys moiety of the compound.

In any aspect, the compound of the invention as described herein is the L diastereomer around the chiral centre of the Y moiety.

In any aspect, the compound as described herein is the D diastereomer around the chiral centre of the Y moiety.

In any aspect, a composition as described herein comprises a compound that is the L diastereomer around the chiral centre of the Y moiety.

In any aspect, a composition as described herein comprises a compound that is the D diastereomer around the chiral centre of the Y moiety.

In any aspect, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the Y moiety.

In any aspect, 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the Y moiety.

In any aspect of the present invention, 1%, 5%, 10%, 20%, 30%, 40%, 50%, 60%, 70%, 80%, 90%, 95%, 96%, 97%, 98%, 99% or more than 99% of the compound present in a composition is the R diastereomer around any one or more of the chiral centres denoted * in formula (XX*) or (XX**) of the compound described herein.

In any aspect of the present invention, 1%, 5%, 10%, 20%, 30%, 40%, 50%, 60%, 70%, 80%, 90%, 95%, 96%, 97%, 98%, 99% or more than 99% of the compound present in a composition is the S diastereomer around any one or more of the chiral centred denoted * in formula (XX*) or (XX**) of the compound described herein.

In any aspect, any of the compounds described herein may be administered in the form of a pharmaceutically acceptable salt.

The term “pharmaceutically acceptable” may be used to describe any pharmaceutically acceptable salt, hydrate or prodrug, or any other compound which upon administration to a subject, is capable of providing (directly or indirectly) a compound of the invention as described herein, or a pharmaceutically acceptable salt, prodrug or ester thereof, or an active metabolite or residue thereof.

Suitable pharmaceutically acceptable salts may include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts may include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

In the case of compounds that are solids, it will be understood by those skilled in the art that the inventive compounds, agents and salts may exist in different crystalline or polymorphic forms, all of which are intended to be within the scope of the present invention and specified formulae.

The term “polymorph” includes any crystalline form of compounds of the invention as described herein, such as anhydrous forms, hydrous forms, solvate forms and mixed solvate forms.

Compounds of the invention described herein are intended to cover, where applicable, solvated as well as unsolvated forms of the compounds. Thus compounds of the invention described herein include compounds having the indicated structures, including the hydrated or solvated forms, as well as the non-hydrated and non-solvated forms.

As used herein, the term “solvate” refers to a complex of variable stoichiometry formed by a solute (in this invention, a compound of the invention described herein, or a pharmaceutically acceptable salt, prodrug or ester thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

The compounds as described herein are to also include isotope variations, such as the replacement of hydrogen for deuterium.

A “prodrug” is a compound that may not fully satisfy the structural requirements of the compounds provided herein, but is modified in vivo, following administration to a subject or patient, to produce a compound of the invention as described herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of any of compounds of Formulas (I)-(XXIII). The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of the compounds described herein, including the compounds of formulas (I)-(XXIII), or other structure as depicted herein.

The general chemical terms used in the formulae herein have their usual meaning.

The term “aliphatic” is intended to include saturated and unsaturated, nonaromatic, straight chain, branched, acyclic, and cyclic hydrocarbons. Those skilled in the art will appreciate that aliphatic groups include, for example, alkyl, alkenyl, alkynyl, cycloalkyl, and cycloalkenyl groups, and hybrids thereof such as (cycloalkyl)alkyl, (cycloalkenyl)alkyl and (cycloalkyl)alkenyl groups. In various embodiments, aliphatic groups comprise from 1-12, 1-8, 1-6, or 1-4 carbon atoms. In some embodiments, aliphatic groups comprise 5-21, from 9-21, or from 11-21 carbon atoms, such as from 11, 13, 15, 17, or 19 carbon atoms. In some embodiments, the aliphatic group is saturated.

The term “heteroaliphatic” is intended to include aliphatic groups, wherein one or more chain and/or ring carbon atoms are independently replaced with a heteroatom, preferably a heteroatom selected from oxygen, nitrogen and sulfur. In some embodiments, the heteroaliphatic is saturated. Examples of heteroaliphatic groups include linear or branched, heteroalkyl, heteroalkenyl, and heteroalkynyl groups.

The term “alkyl” is intended to include saturated straight chain and branched chain hydrocarbon groups. In some embodiments, alkyl groups have from 1 to 12, 1 to 10, 1 to 8, 1 to 6, or from 1 to 4 carbon atoms. In some embodiments, alkyl groups have from 5-21, from 9-21, or from 11-21 carbon atoms, such as from 11, 13, 15, 17, or 19 carbon atoms. Examples of straight chain alkyl groups include, but are not limited to, methyl, ethyl, n-propyl, n-butyl, n-pentyl, n-hexyl, n-heptyl, and n-octyl. Examples of branched alkyl groups include, but are not limited to, isopropyl, iso-butyl, sec-butyl, tert-butyl, neopentyl, isopentyl, and 2,2-dimethylpropyl.

The term “alkenyl” is intended to include straight and branched chain alkyl groups having at least one double bond between two carbon atoms. In some embodiments, alkenyl groups have from 2 to 12, from 2 to 10, from 2 to 8, from 2 to 6, or from 2 to 4 carbon atoms. In some embodiments, alkenyl groups have from 5-21, from 9-21, or from 11-21 carbon atoms, such as from 11, 13, 15, 17, or 19 carbon atoms. In some embodiments, alkenyl groups have one, two, or three carbon-carbon double bonds. Examples of alkenyl groups include, but are not limited to, vinyl, allyl, —CH═CH(CH₃), —CH═C(CH₃)₂, —C(CH₃)═CH₂, and —C(CH₃)═CH(CH₃).

The term “alkynyl” is intended to include straight and branched chain alkyl groups having at least one triple bond between two carbon atoms. In some embodiments, the alkynyl group have from 2 to 12, from 2 to 10, from 2 to 8, from 2 to 6, or from 2 to 4 carbon atoms. In some embodiments, alkynyl groups have one, two, or three carbon-carbon triple bonds. Examples include, but are not limited to, —C═CH, —C═CH₃, —CH₂C═CH₃, and —C═CH₂CH(CH₂CH₃)₂.

The term “heteroalkyl” is intended to include alkyl groups, wherein one or more chain carbon atoms are replaced with a heteroatom, preferably a heteroatom selected from the group consisting of oxygen, nitrogen, and sulfur. In some embodiments, the heteroalkyl is saturated. Heteroalkyl groups include, for example, polyethylene glycol groups and polyethylene glycol ether groups, and the like.

The term “cycloalkyl” is intended to include mono-, bi- or tricyclic alkyl groups. In some embodiments, cycloalkyl groups have from 3 to 12, from 3 to 10, from 3 to 8, from 3 to 6, from 3 to 5 carbon atoms in the ring(s). In some embodiments, cycloalkyl groups have 5 or 6 ring carbon atoms. Examples of monocyclic cycloalkyl groups include, but are not limited to, cyclopropyl, cyclobutyl, cyclopentyl, cyclohexyl, cycloheptyl, and cyclooctyl. In some embodiments, the cycloalkyl group has from 3 to 8, from 3 to 7, from 3 to 6, from 4 to 6, from 3 to 5, or from 4 to 5 ring carbon atoms. Bi- and tricyclic ring systems include bridged, spiro, and fused cycloalkyl ring systems. Examples of bi- and tricyclic ring cycloalkyl systems include, but are not limited to, bicyclo[2.1.1]hexanyl, bicyclo[2.2.1]heptanyl, adamantyl, and decalinyl.

The term “cycloalkenyl” is intended to include non-aromatic cycloalkyl groups having at least one double bond between two carbon atoms. In some embodiments, cycloalkenyl groups have one, two or three double bonds. In some embodiments, cycloalkenyl groups have from 4 to 14, from 5 to 14, from 5 to 10, from 5 to 8, or from 5 to 6 carbon atoms in the ring(s). In some embodiments, cycloalkenyl groups have 5, 6, 7, or 8 ring carbon atoms. Examples of cycloalkenyl groups include cyclohexenyl, cyclopentenyl, cyclohexadienyl, butadienyl, pentadienyl, and hexadienyl.

The term “aryl” is intended to include cyclic aromatic hydrocarbon groups that do not contain any ring heteroatoms. Aryl groups include monocyclic, bicyclic and tricyclic ring systems. Examples of aryl groups include, but are not limited to, phenyl, azulenyl, heptalenyl, biphenyl, fluorenyl, phenanthrenyl, anthracenyl, indenyl, indanyl, pentalenyl, and naphthyl. In some embodiments, aryl groups have from 6 to 14, from 6 to 12, or from 6 to 10 carbon atoms in the ring(s). In some embodiments, the aryl groups are phenyl or naphthyl. Aryl groups include aromatic-aliphatic fused ring systems. Examples include, but are not limited to, indanyl and tetrahydronaphthyl.

The term “heterocyclyl” is intended to include non-aromatic ring systems containing 3 or more ring atoms, of which one or more is a heteroatom. In some embodiments, the heteroatom is nitrogen, oxygen, or sulfur. In some embodiments, the heterocyclyl group contains one, two, three, or four heteroatoms. In some embodiments, heterocyclyl groups include mono-, bi- and tricyclic rings having from 3 to 16, from 3 to 14, from 3 to 12, from 3 to 10, from 3 to 8, or from 3 to 6 ring atoms. Heterocyclyl groups include partially unsaturated and saturated ring systems, for example, imidazolinyl and imidazolidinyl. Heterocyclyl groups include fused and bridged ring systems containing a heteroatom, for example, quinuclidyl. Heterocyclyl groups include, but are not limited to, aziridinyl, azetidinyl, azepanyl, diazepanyl, 1,3-dioxanyl, 1,3-dioxolanyl, isoxazolidinyl, morpholinyl, piperazinyl, piperidinyl, pyranyl, pyrazolidinyl, pyrrolinyl, pyrrolidinyl, tetrahydrofuranyl, tetrahydrothienyl, thiadiazolidinyl, and trithianyl.

The term “heteroaryl” is intended to include aromatic ring systems containing 5 or more ring atoms, of which, one or more is a heteroatom. In some embodiments, the heteroatom is nitrogen, oxygen, or sulfur. In some embodiments, heteroaryl groups include mono-, bi- and tricyclic ring systems having from 5 to 16, from 5 to 14, from 5 to 12, from 5 to 10, from 5 to 8, or from 5 to 6 ring atoms. Heteroaryl groups include, but are not limited to, pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, pyrazolopyridinyl, triazolopyridinyl, benzotriazolyl, benzoxazolyl, benzothiazolyl, imidazopyridinyl, isoxazolopyridinylxanthinyl, guaninyl, quinolinyl, isoquinolinyl, tetrahydroquinolinyl, quinoxalinyl, and quinazolinyl. Heteroaryl groups include fused ring systems in which all of the rings are aromatic, for example, indolyl, and fused ring systems in which only one of the rings is aromatic, for example, 2,3-dihydroindolyl.

The term “halo” or “halogen” is intended to include F, Cl, Br, and I.

The term “heteroatom” is intended to include oxygen, nitrogen, sulfur, or phosphorus. In some embodiments, the heteroatom is selected from the group consisting of oxygen, nitrogen, and sulfur.

As used herein, the term “substituted” is intended to mean that one or more hydrogen atoms in the group indicated is replaced with one or more independently selected suitable substituents, provided that the normal valency of each atom to which the substituent(s) are attached is not exceeded, and that the substitution results in a stable compound. In some embodiments, optional substituents in the compounds described herein include but are not limited to halo, CN, NO₂, OH, NH₂, NHR₁₀₀, NR₁₀₀R₂₀₀, C₁₋₆haloalkyl, C₁₋₆haloalkoxy, C(O)NH₂, C(O)NHR₁₀₀, C(O)NR₁₀₀R₂₀₀, SO₂R₁₀₀, OR₁₀₀, SR₁₀₀, S(O)R₁₀₀, C(O)R₁₀₀, and C₁₋₆aliphatic; wherein R₁₀₀ and R₂₀₀ are each independently C₁₋₆aliphatic, for example C₁₋₆alkyl.

Where a protecting group (PG) is referred to, a person skilled in the art would readily understand what type of protecting group would be suitable.

The term “amine protecting group” as used herein is intended to mean a group that is capable of being readily removed to provide the NH₂ group of an amine group and protects the amine group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, acyl and acyloxy groups, for example acetyl, chloroacetyl, trichloroacetyl, o-nitrophenylacetyl, o-nitrophenoxy-acetyl, trifluoroacetyl, acetoacetyl, 4-chlorobutyryl, isobutyryl, picolinoyl, aminocaproyl, benzoyl, methoxy-carbonyl, 9-fluorenylmethoxycarbonyl, 2,2,2-trifluoroethoxycarbonyl, 2-trimethylsilylethoxy-carbonyl, tert-butyloxycarbonyl, benzyloxycarbonyl, p-nitrobenzyloxycarbonyl, 2,4-dichloro-benzyloxycarbonyl, and the like. Further examples include Cbz (carboxybenzyl), Nosyl (o- or p-nitrophenylsulfonyl), Bpoc (2-(4-biphenyl)isopropoxycarbonyl) and Dde (1-(4,4-dimethyl-2,6-dioxohexylidene)ethyl). In some embodiments, the amine protecting groups for the purposes described herein include (but are not limited to) tert-butyloxycarbonyl (t-Boc) and 9H-fluoren-9-ylmethoxycarbonyl (Fmoc).

The term “carboxyl protecting group” as used herein is intended to mean a group that is capable of being readily removed to provide the OH group of a carboxyl group and protects the carboxyl group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, alkyl and silyl groups, for example methyl, ethyl, tert-butyl, methoxymethyl, 2,2,2-trichloroethyl, benzyl, diphenylmethyl, trimethylsilyl, and tert-butyldimethylsilyl, and the like.

The term “carboxamide protecting group” as used herein is intended to mean a group that is capable of being readily removed to provide the NH₂ group of a carboxamide group and protects the carboxamide group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, 9-xanthenyl (Xan), trityl (Trt), methyltrityl (Mtt), cyclopropyldimethylcarbinyl (Cpd), and dimethylcyclopropylmethyl (Dmcp).

The term “ester” refers to a carboxylic acid group where the hydrogen of the hydroxyl group has been replaced by a saturated, straight-chain (i.e. linear) or branched hydrocarbon group. Specific examples of alkyl groups are methyl, ethyl, propyl, iso-propyl, n-butyl, iso-butyl, sec-butyl, tert-butyl, n-pentyl, iso-pentyl, n-hexyl and 2,2-dimethylbutyl. The alkyl group may be a C₁-C₆ alkyl group. As used herein a wording defining the limits of a range of length such as, for example, “from 1 to 5” means any integer from 1 to 5, i.e. 1, 2, 3, 4 and 5. In other words, any range defined by two integers explicitly mentioned is meant to comprise and disclose any integer defining said limits and any integer comprised in said range. The alkyl group may be a branched alkyl group.

As used herein, ‘Ser’ refers to the amino acid serine and ‘Cys’ refers to the amino acid cysteine.

As used herein, ‘PEG’ refers to the polymer compound polyethylene glycol. Unless otherwise defined, reference to ‘PEG’ includes any length polymer of ethylene oxide. Reference to PEG also includes substituted PEG. In some embodiments, substituted PEG may be defined by formulas B-I or B-II as described herein.

As used herein, the term “and/or” means “and”, or “or”, or both.

The term “(s)” following a noun contemplates the singular and plural form, or both.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In some embodiments, the compound comprising a TLR2 agonist moiety conjugated with a solubilising moiety may be any of compounds 001-010, A101-A118, A201-A232 and B1-B16, or a pharmaceutically acceptable salt, solvate, polymorph and/or prodrug thereof:

| | Compound |
|---|---|
| Compound Structure | name |
| 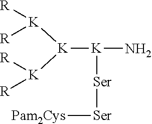 | 001 |
| 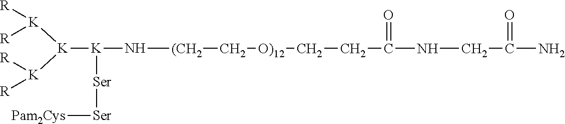 | 002 |
| 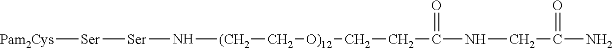 | 003 |
| Pam₂Cys—Ser—Ser—Lys—Lys—Lys—Lys | 004 |
| Pam₂Cys—Ser—Lys—Lys—Lys—Lys | 005 |
| 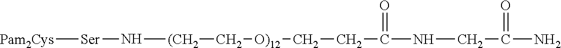 | A101/ compound 1 |
| 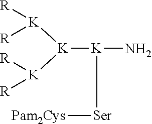 | 007 |
| 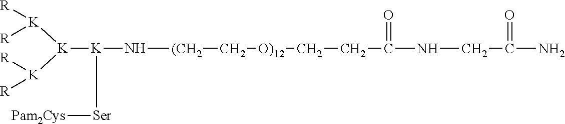 | 008 |
| 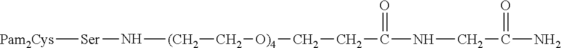 | 009 |
| 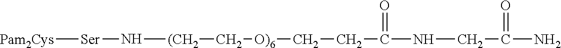 | 010 |
| 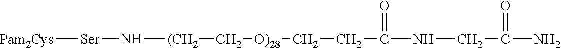 | A105 |
| 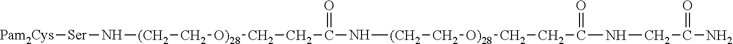 | A106 |
| 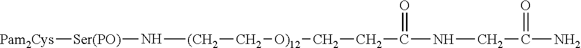 | A104 |
| 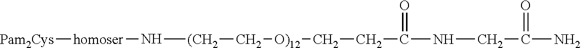 | A103 |
| 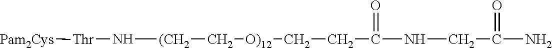 | A102 |
| 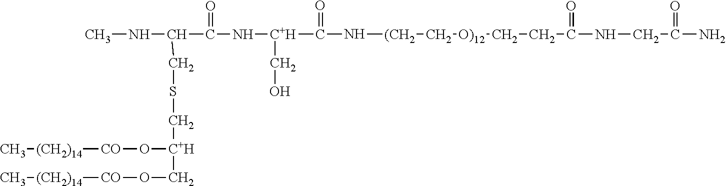 | A109 |
| 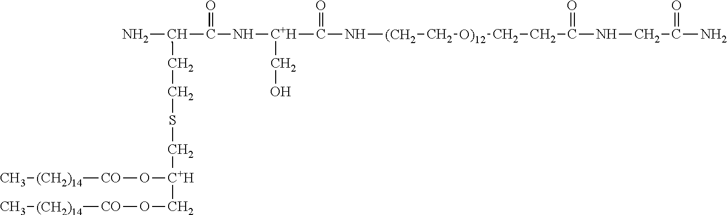 | A110 |
| 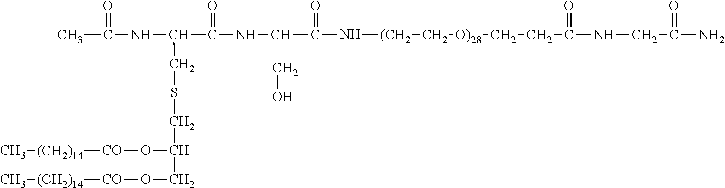 | A111 |
| 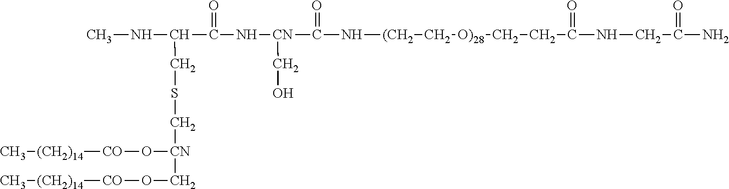 | A112 |
| 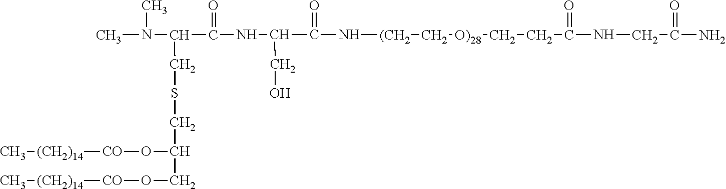 | A113 |
| 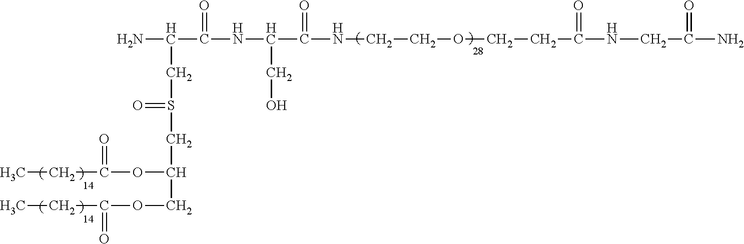 | A114 |
| 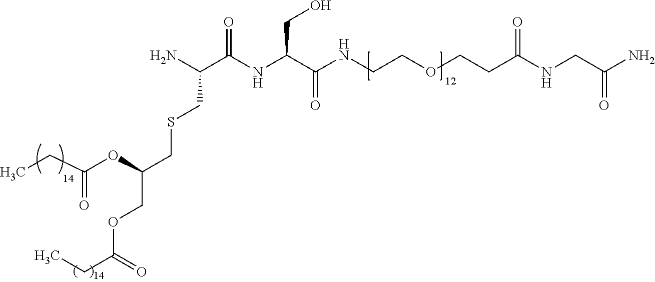 | A107 |
| 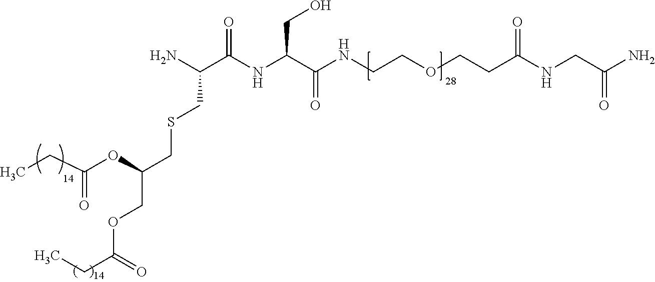 | A108 |
| 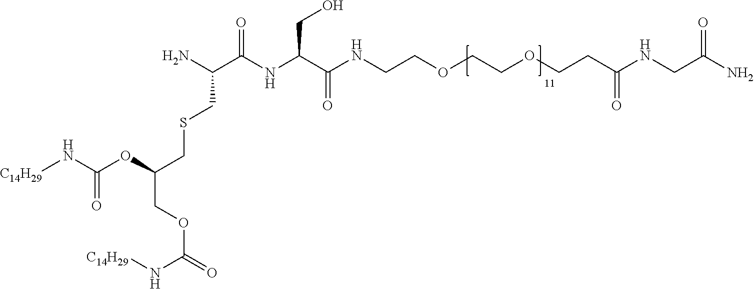 | A115 |
| 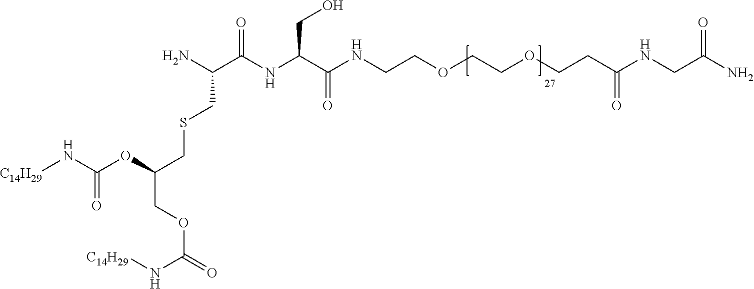 | A116 |
| 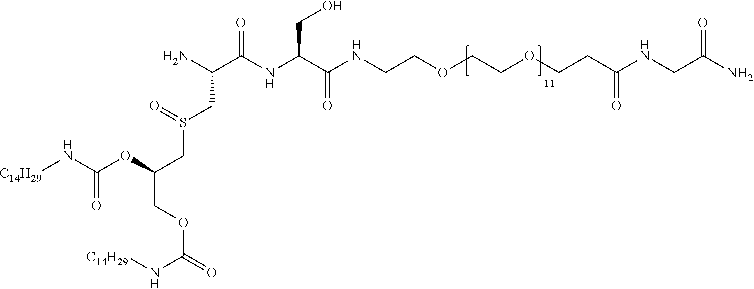 | A117 |
| 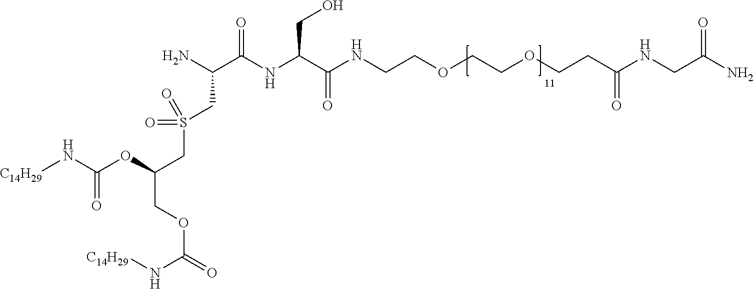 | A118 |
| 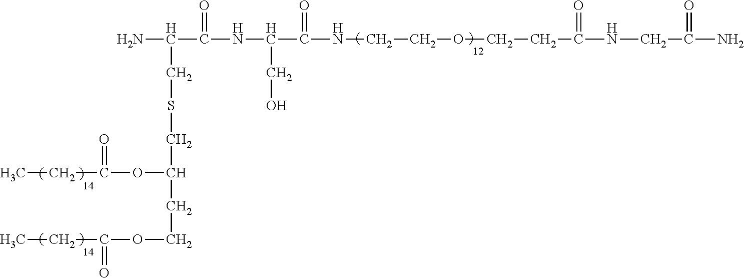 | A201 |
| 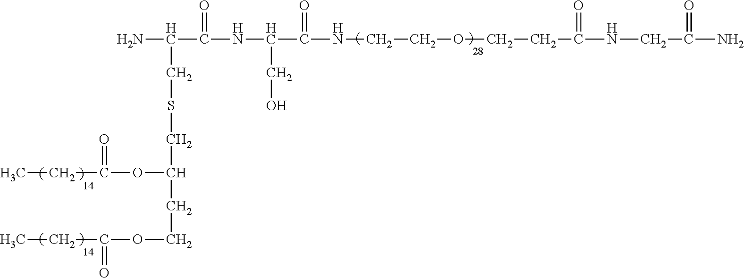 | A202 |
| 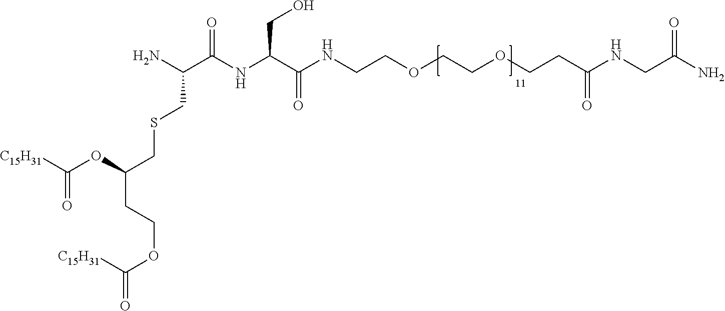 | A203 |
| 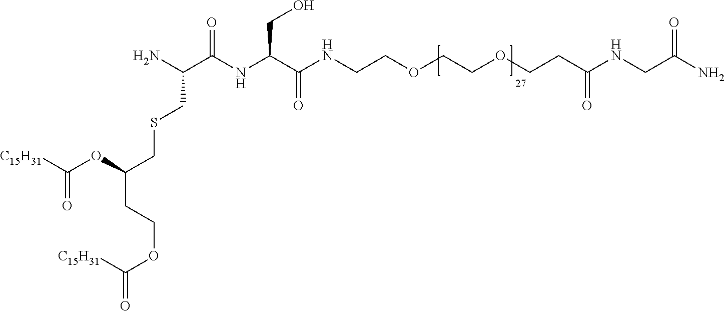 | A204 |
| 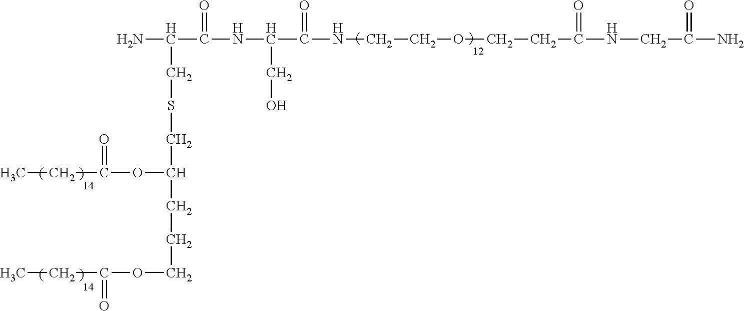 | A205 |
| 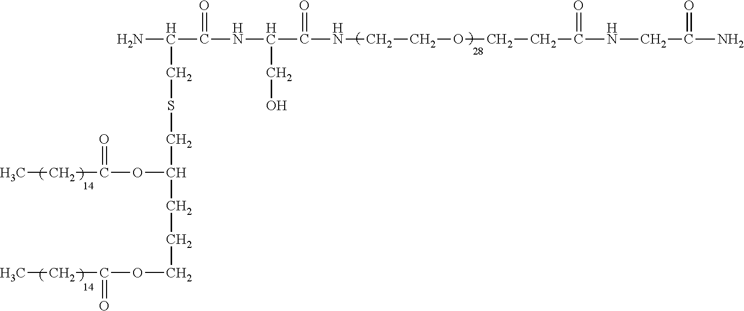 | A206 |
| 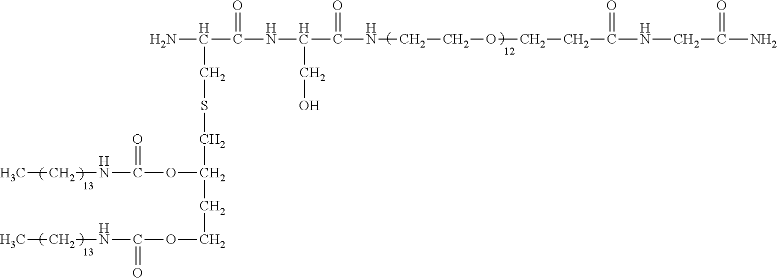 | A207 |
| 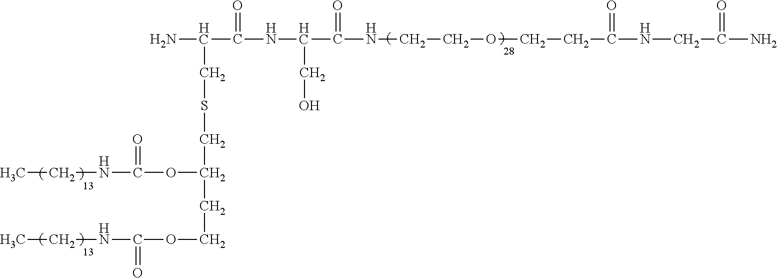 | A208 |
| 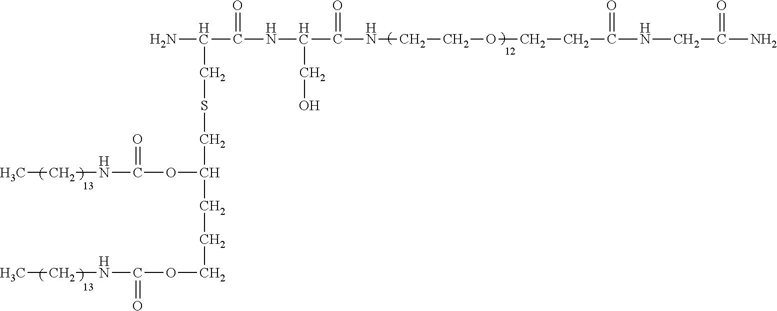 | A209 |
| 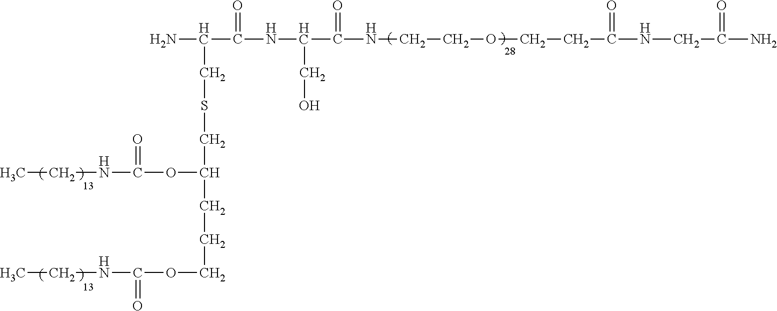 | A210 |
| 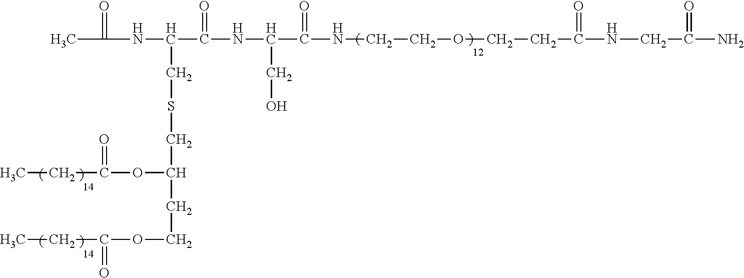 | A211 |
| 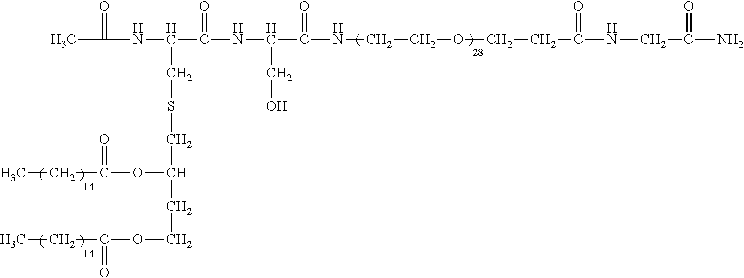 | A212 |
| 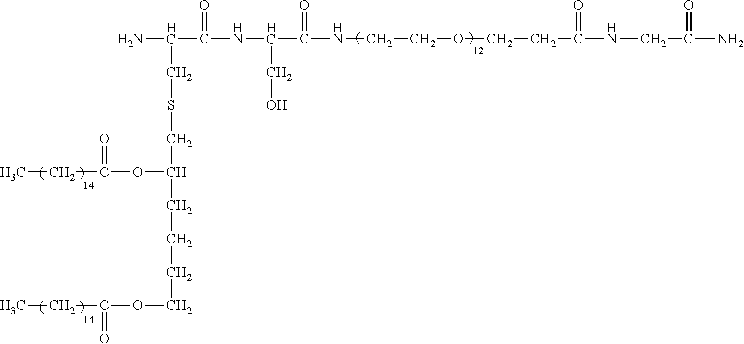 | A213 |
| 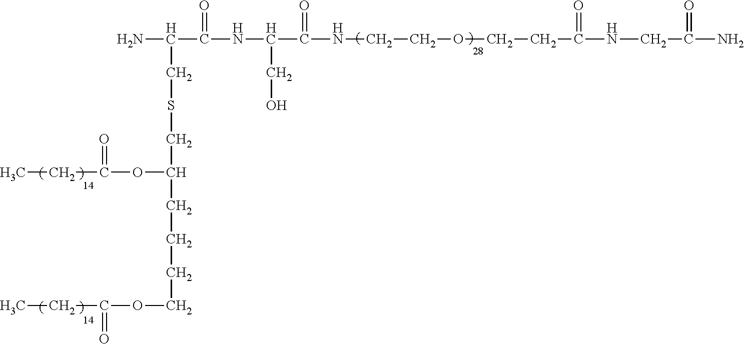 | A214 |
| 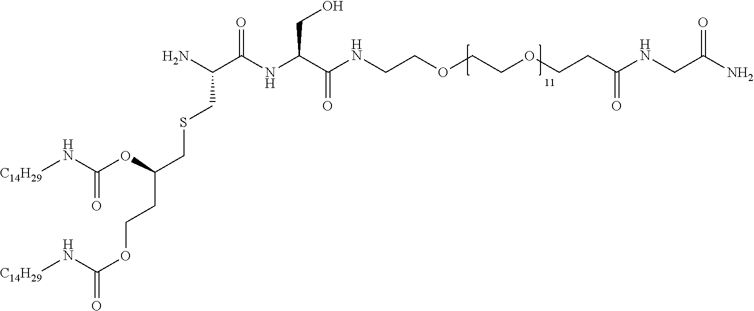 | A215 |
| 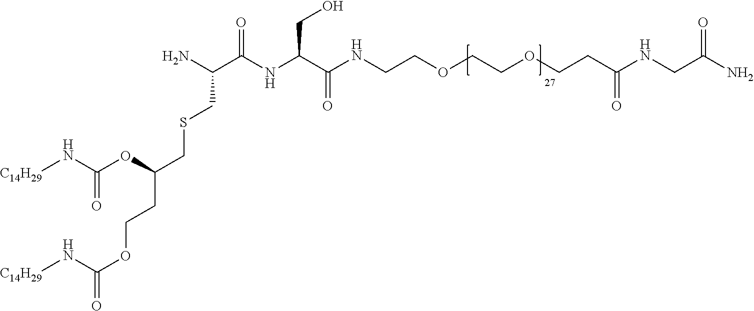 | A216 |
| 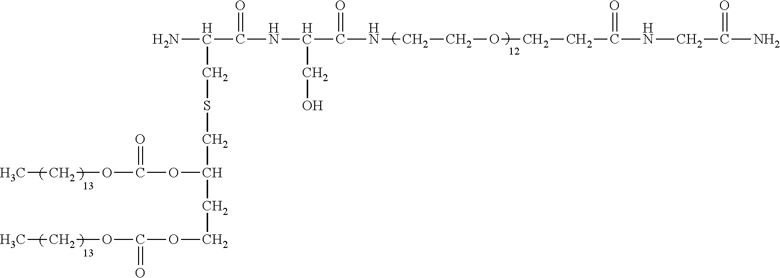 | A217 |
| 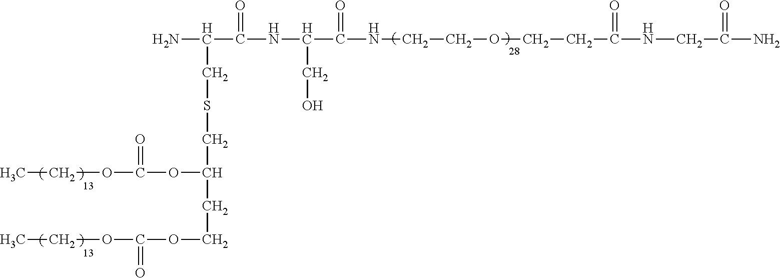 | A218 |
| 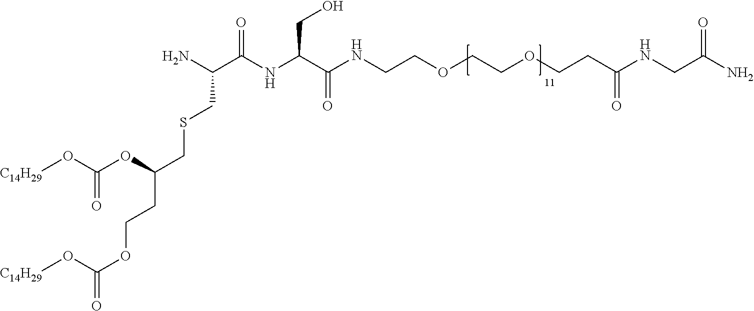 | A219 |
| 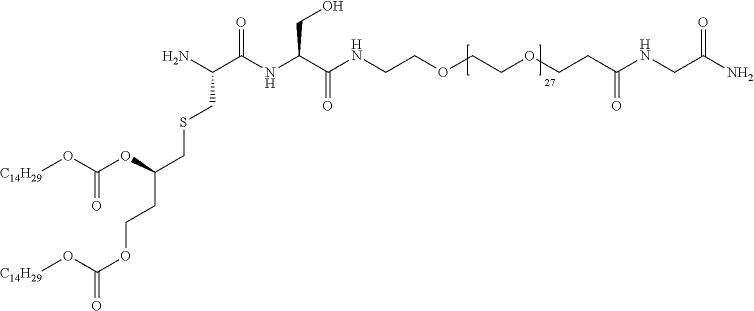 | A220 |
| 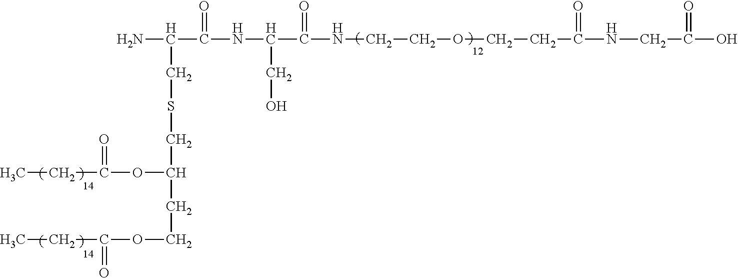 | A221 |
| 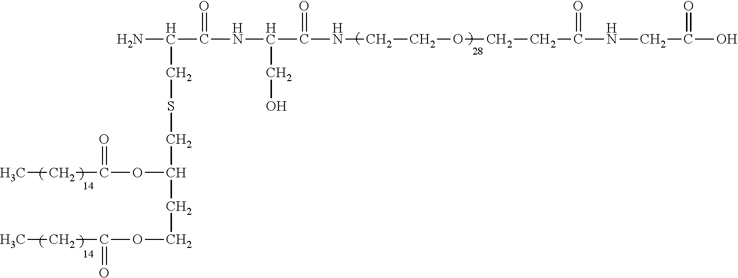 | A222 |
| 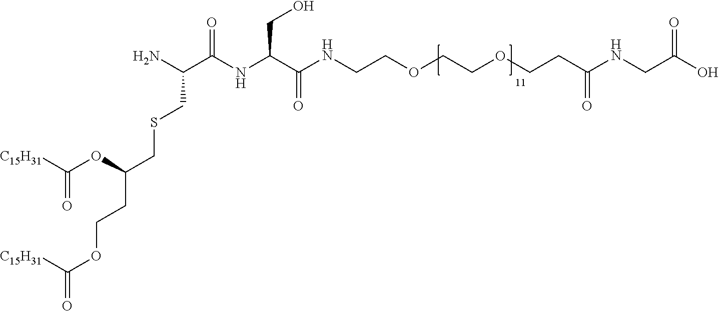 | A223 |
| 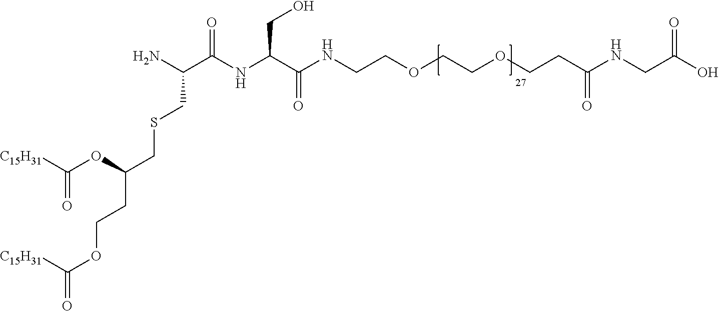 | A224 |
| 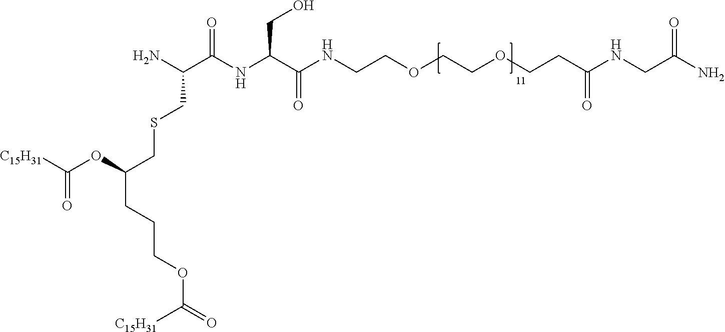 | A225 |
| 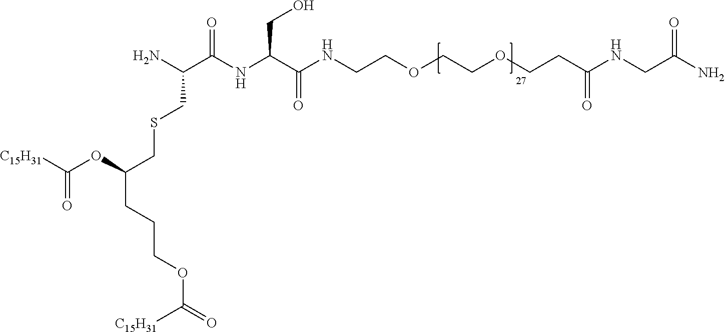 | A226 |
| 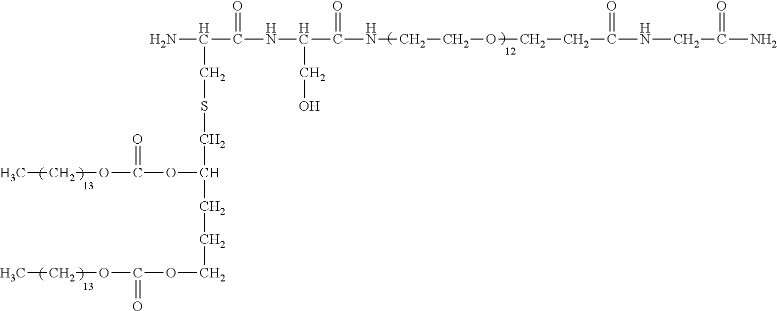 | A227 |
| 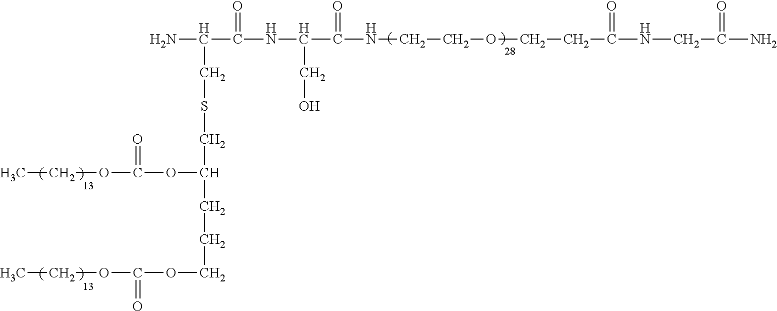 | A228 |
| 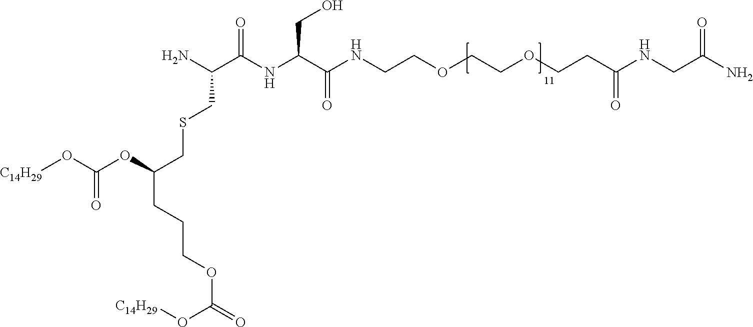 | A229 |
| 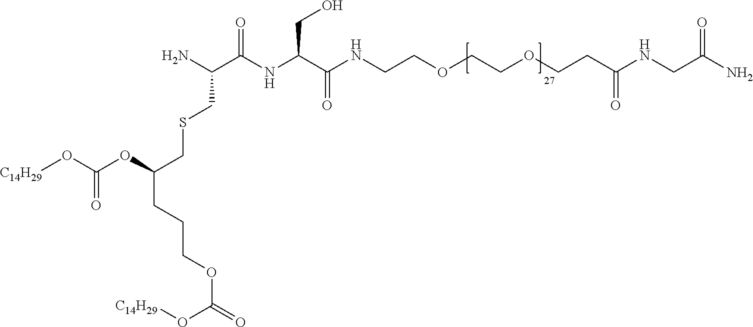 | A230 |
| 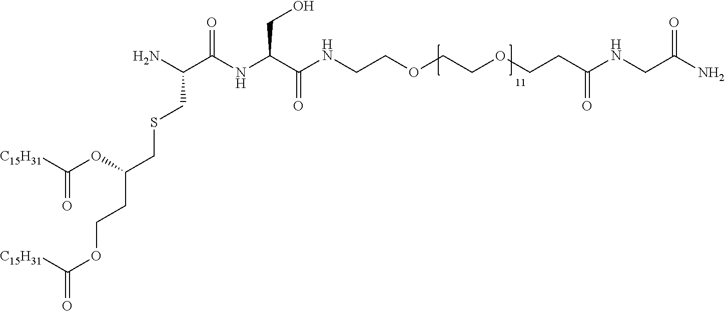 | A231 |
| 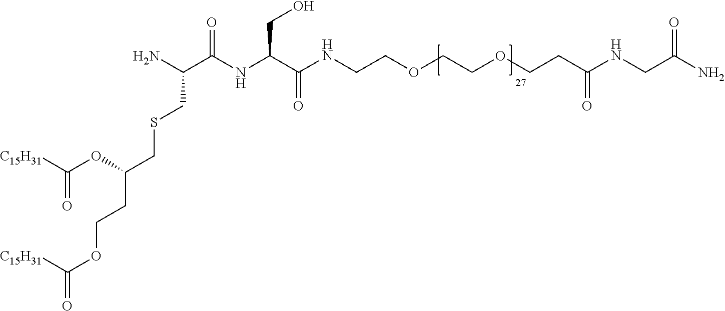 | A232 |
| 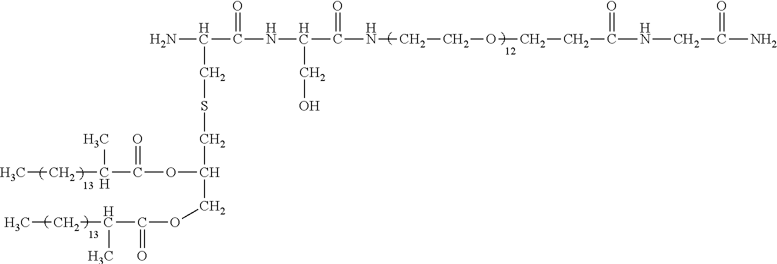 | B1 |
| 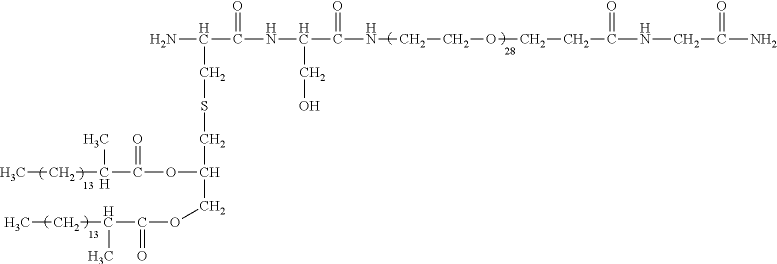 | B2 |
| 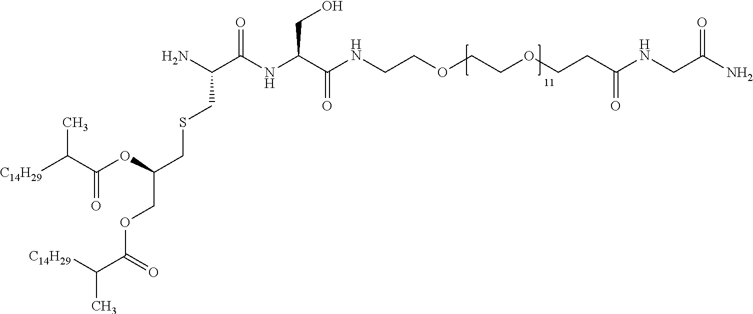 | B3 |
| 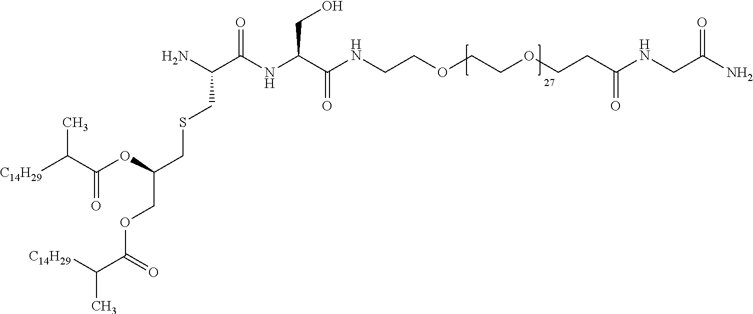 | B4 |
| 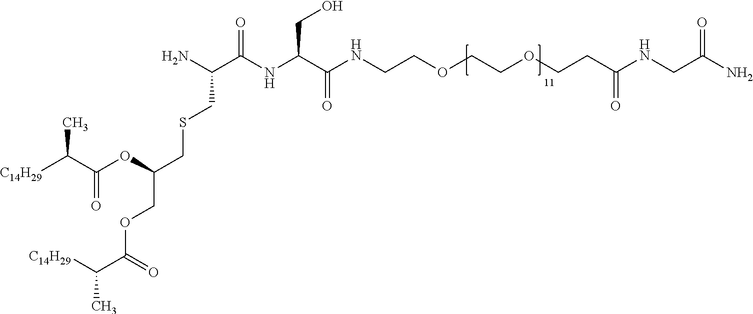 | B5 |
| 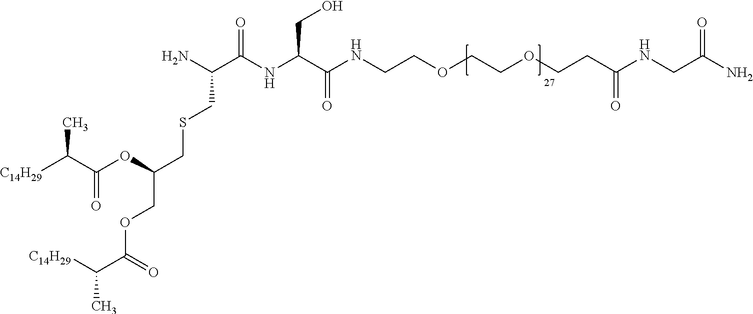 | B6 |
| 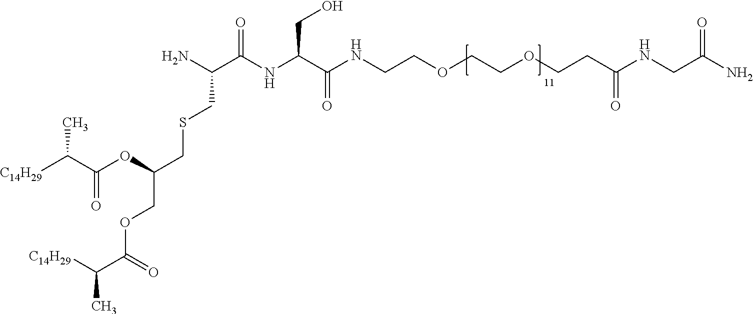 | B7 |
| 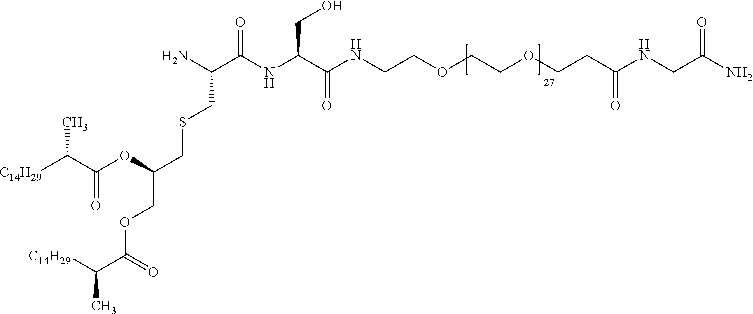 | B8 |
| 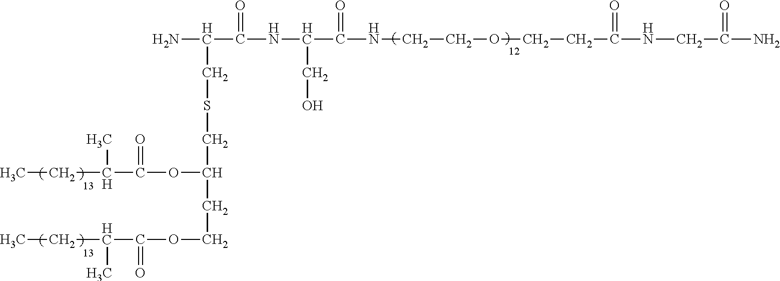 | B9 |
| 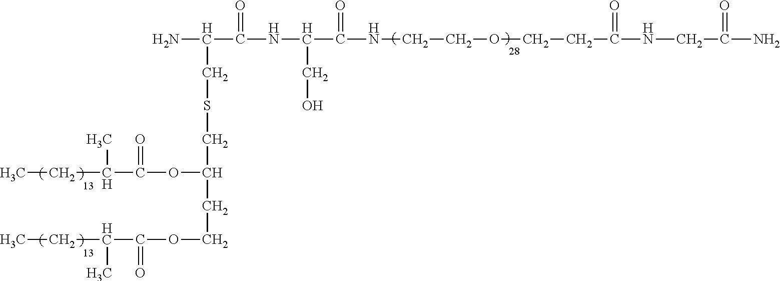 | B10 |
| 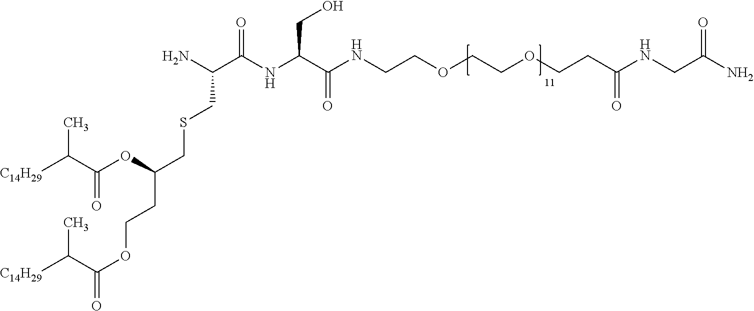 | B11 |
| 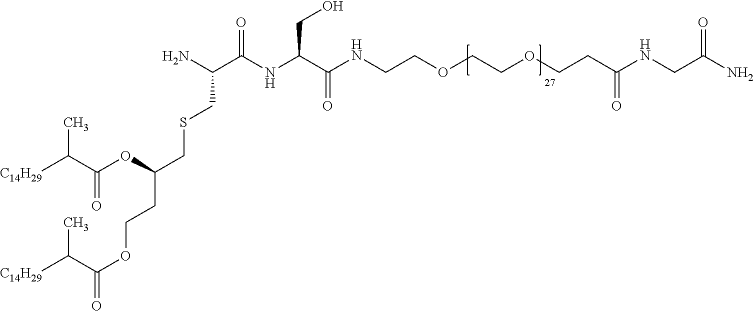 | B12 |
| 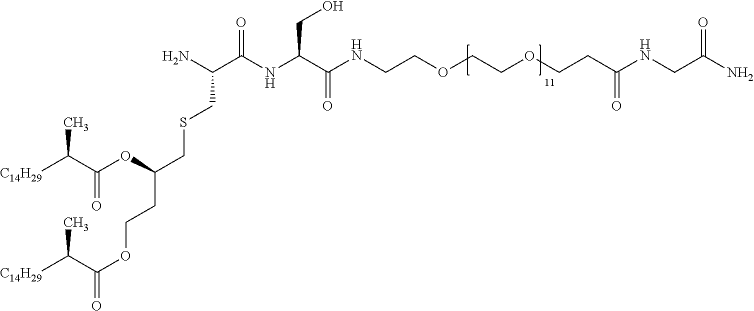 | B13 |
| 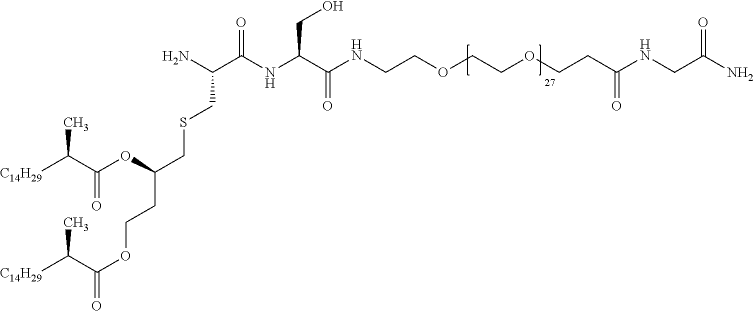 | B14 |
| 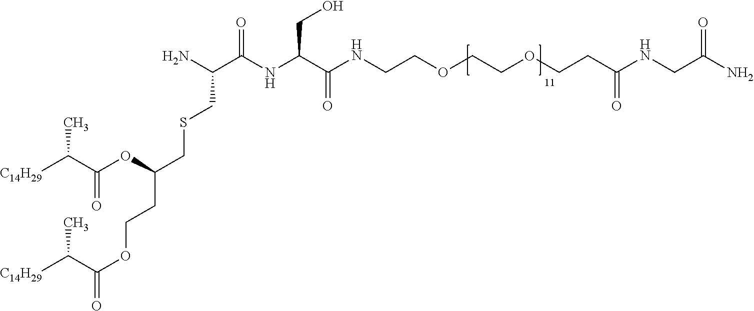 | B15 |
| 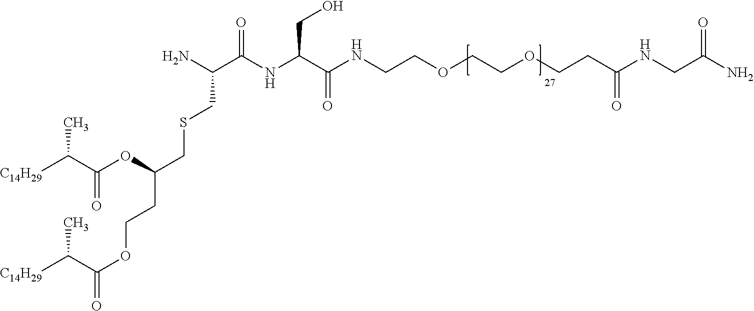 | B16 |

In some embodiments, the compound may be selected from A101-A118, A201-A232 and B1-B16, or a pharmaceutically acceptable salt, solvate, polymorph and/or prodrug thereof.

Compounds of the invention may be prepared by techniques known in the art. For example, compounds of the invention including any one of formulas (I)-(XXIII) comprising an A1 moiety may be prepared by techniques described in WO2019/119067 (US 2021/0230217 A1), compounds of the invention including any one of formulas (I)-(XXIII) comprising an A2 moiety may be prepared by techniques described in WO 2020/257870; and compounds of the invention including any one of formulas (XX)-(XXII) may be prepared by techniques described in international application no. PCT/AU2021/050667.

The pharmaceutical compositions may additionally comprise a pharmaceutically acceptable carrier, diluent or excipient.

### TLR2 Agonist Treatments

As discussed above, the present invention provides formulations of Toll-Like Receptor 2 protein (TLR2) agonist compounds. In humans, TLR2 plays a fundamental role in the recognition of pathogens and activation of the innate immunity response. It is encoded by the TLR2 gene and is expressed on the surface of specific cells.

Without wishing to be bound by any theory or mode of action, it is believed that the compounds of the invention described herein are agonists of TLR2 and show activity by binding at TLR2 and stimulating the innate immune system. The innate immune system forms an immediate defence against pathogens such as pathogens that infect and replicate in cells lining the respiratory tract. Research has shown that agents which stimulate the innate immune system may be useful for limiting respiratory infections, which may provide protection from infections both in isolation and during the period between inoculation and the formation of antibodies and immune cells. Such agents are considered to be useful for the treatment and/or prevention of respiratory infections, or respiratory conditions caused by or associated with infectious agents such as a virus (such as Influenza A or coronavirus) or bacterium (such as pneumonia) in a non-antigen specific manner.

In one aspect, therefore, the present invention provides a method of treating and/or preventing a disease, comprising raising an innate immune response in a subject by administering an effective amount of a pharmaceutical composition of the invention as described herein to the subject in need thereof.

In another aspect, the present invention provides a method of treating and/or preventing a disease caused by an infectious agent, comprising administering to a subject in need thereof an effective amount of a pharmaceutical composition of the invention as described herein.

In another aspect, the present invention provides a method of treating and/or preventing a respiratory disease or condition associated with a viral or bacterial infection, comprising administering to a subject in need thereof a pharmaceutical composition of the invention as described herein.

In another aspect, the present invention provides a method of treating and/or preventing a respiratory infection, comprising administering to a subject in need thereof a pharmaceutical composition of the invention as described herein. Preferably the method further comprises a step of identifying a subject having a respiratory infection.

In another aspect, the present invention provides a method for reducing airway inflammation, comprising administering to a subject in need thereof a pharmaceutical composition of the invention as described herein.

The present invention also provides a method of improving the ability of a subject to control a respiratory disease or condition during a respiratory viral infection, the method comprising administering to a subject in need thereof a pharmaceutical composition of the invention as described herein. Preferably the infection is not a rhinovirus infection.

The present invention also provides a method of treating and/or preventing a disease or condition associated with the TLR2 receptor, the method comprising administering to a subject in need thereof a pharmaceutical composition of the invention as described herein.

The present invention also provides a method of agonising TLR2 activity in a cell, the method comprising contacting the cell with a pharmaceutical composition of the invention as described herein. In some embodiments, the cell is contacted with a pharmaceutical composition of the invention as described herein. In some embodiments, the cell is provided in the form of a cell culture.

In another aspect, the present invention provides for use of a compound of the invention as described herein or a pharmaceutically acceptable salt, solvate or prodrug thereof in the preparation of a medicament for raising an innate immune response in a subject.

In another aspect, the present invention provides for use of a compound of the invention as described herein or a pharmaceutically acceptable salt, solvate or prodrug thereof in the preparation of a medicament for treating and/or preventing a disease caused by an infectious agent.

In another aspect, the present invention further provides for use of a compound of the invention as described herein or a pharmaceutically acceptable salt, solvate or prodrug thereof in the preparation of a medicament for treating and/or preventing a respiratory disease or condition associated with a viral or bacterial infection in a subject.

In another aspect, the present invention further provides for use of a compound of the invention as described herein or a pharmaceutically acceptable salt, solvate or prodrug thereof in the preparation of a medicament for treating and/or preventing a respiratory infection in a subject.

In yet another aspect, the present invention provides for use of a compound of the invention as described herein or a pharmaceutically acceptable salt, solvate or prodrug thereof in the preparation of a medicament for treating and/or preventing a respiratory infection.

In another aspect, the present invention further provides use of a compound of the invention as described herein or a pharmaceutically acceptable salt, solvate or prodrug thereof in the preparation of a medicament for reducing airway inflammation.

In another aspect, the present invention further provides use of a compound of the invention as described herein or a pharmaceutically acceptable salt, solvate or prodrug thereof in the preparation of a medicament for improving the ability of a subject to control a respiratory disease or condition during a respiratory viral infection.

In another aspect, the present invention further provides use of a compound of the invention as described herein or a pharmaceutically acceptable salt, solvate or prodrug thereof in the preparation of a medicament for treating and/or preventing a disease or condition associated with the TLR2 receptor.

In one aspect, the present invention provides for use of a compound of the invention as described herein or a pharmaceutically acceptable salt, solvate or prodrug thereof, for raising an innate immune response in a subject.

In another aspect, the present invention provides for use of a compound of the invention as described herein or a pharmaceutically acceptable salt, solvate or prodrug thereof, for preventing a disease caused by an infectious agent, in a subject.

In another aspect, the present invention provides for use of a compound of the invention as described herein or a pharmaceutically acceptable salt, solvate or prodrug thereof, for treating and/or preventing a respiratory disease or condition associated with a viral or bacterial infection in a subject.

In a further aspect, the invention provides for use of a compound of the invention as described herein or a pharmaceutically acceptable salt, solvate or prodrug thereof, for (a) treating and/or preventing a respiratory infection in a subject; (b) reducing airway inflammation in a subject; (c) controlling a respiratory disease or condition during a respiratory viral infection in a subject; (d) for treating and/or preventing a disease or condition associated with the TLR2 receptor.

In any aspect of the invention, the compound of the invention as described herein or a pharmaceutically acceptable salt, solvate or prodrug thereof may be conjugated with other compounds. Other compounds are any of those described herein.

In any aspect of the invention, the a pharmaceutical composition of the invention as described herein is administered once daily or once weekly.

In any aspect of the invention, where prevention or prophylaxis is intended or required, the composition is administered to the subject before any clinically or biochemically detectable symptoms of viral infection.

In any aspect of the invention, administration of the a pharmaceutical composition of the invention as described herein to a subject reduces viral load in a subject. Preferably, the viral load is reduced in the respiratory tract, for example the upper and/or lower respiratory tract. Preferably, the viral load is reduced in the lungs.

In any aspect herein, the infectious agent may be a virus. Preferably, the virus is one associated with infection of the respiratory tract. Even more preferably, the virus is influenza, rhinovirus or coronavirus, or any other virus described herein.

Influenza (commonly referred to as “the flu”) is an infectious disease caused by RNA viruses of the family Orthomyxoviridae (the influenza viruses) that affects birds and mammals. The most common symptoms of the disease are chills, fever, sore throat, muscle pains, severe headache, coughing, weakness/fatigue and general discomfort.

The influenza viruses make up three of the five genera of the family Orthomyxoviridae. Influenza Type A and Type B viruses co-circulate during seasonal epidemics and can cause severe influenza infection. Influenza Type C virus infection is less common but can be severe and cause local epidemics.

Influenza Type A virus can be subdivided into different serotypes or subtypes based on the antibody response to these viruses. Influenza A viruses are divided into subtypes based on two proteins on the surface of the virus: the hemagglutinin (H) and the neuraminidase (N). There are 18 different hemagglutinin subtypes and 11 different neuraminidase subtypes. (H1 through H18 and N1 through N11 respectively.) The sub types that have been confirmed in humans are H1N1, H1N2, H2N2, H3N2, H5N1, H7N2, H7N3, H7N7, H9N2 and H10N7.

Influenza has an enormous impact on public health with severe economic implications in addition to the devastating health problems, including morbidity and even mortality. Accordingly, there is a need for therapeutic agents which can prevent infection, or reduce severity of infection in individuals.

In any aspect or embodiment of the invention, the influenza infection for which treatment or prevention is required is an infection with a virus selected from the group consisting of influenza Types A, B or C.

“Coronavirus” as used herein refers members of the subfamily Coronavirinae in the family Coronaviridae and the order Nidovirales (International Committee on Taxonomy of Viruses). This subfamily consists of four genera, Alphacoronavirus, Betacoronavirus, Gammacoronavirus and Deltacoronavirus, on the basis of their phylogenetic relationships and genomic structures. Subgroup clusters are labeled as 1a and 1b for the Alphacoronavirus and 2a, 2b, 2c, and 2d for the Betacoronavirus. The alphacoronaviruses and betacoronaviruses infect only mammals. The gammacoronaviruses and deltacoronaviruses infect birds, but some of them can also infect mammals. Alphacoronaviruses and betacoronaviruses usually cause respiratory illness in humans and gastroenteritis in animals. The three highly pathogenic viruses, SARS-CoV, MERS-CoV and SARS-CoV2, cause severe respiratory syndrome in humans, and the other four human coronaviruses (HCoV-NL63, HCoV-229E, HCoV-OC43 and HKU1) induce only mild upper respiratory diseases in immunocompetent hosts, although some of them can cause severe infections in infants, young children and elderly individuals. Alphacoronaviruses and betacoronaviruses can pose a heavy disease burden on livestock; these viruses include porcine transmissible gastroenteritis virus, porcine enteric diarrhoea virus (PEDV) and the recently emerged swine acute diarrhoea syndrome coronavirus (SADS-CoV). On the basis of current sequence databases, all human coronaviruses have animal origins: SARS-CoV, MERS-CoV, SARS-CoV2, HCoV-NL63 and HCoV-229E are considered to have originated in bats; HCoV-OC43 and HKU1 likely originated from rodents.

The coronaviruses include antigenic groups I, II, and Ill. Nonlimiting examples of coronaviruses include SARS coronavirus, MERS coronavirus, transmissible gastroenteritis virus (TGEV), human respiratory coronavirus, porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus, as well as any others described herein, and including those referred to in Cui, et al. Nature Reviews Microbiology volume 17, pages 181-192 (2019), and Shereen et al. Journal of Advanced Research, Volume 24, July 2020 (published online 16 Mar. 2020), Pages 91-98.

In any aspect of the present invention, the coronavirus may be from any of the genera Alphacoronavirus, Betacoronavirus, Gammacoronavirus or Deltacoronavirus. Preferably, the coronavirus is from one of the Alphacoronavirus subgroup clusters 1a and 1b or one of the Betacoronavirus subgroup clusters 2a, 2b, 2c, and 2d. The coronavirus may be any one that infects humans. Exemplary coronaviruses are SARS-CoV, MERS-CoV, SARS-CoV2, HCoV-NL63, HCoV-229E, HCoV-OC43 and HKU1, although the coronavirus may be any one as described herein. Most preferably, the coronavirus is SARS-CoV2.

As used herein, a viral infection may be a coronavirus infection.

The term ‘respiratory disease’ or ‘respiratory condition’ refers to any one of several ailments that involve inflammation and affect a component of the respiratory system including the upper (including the nasal cavity, pharynx and larynx) and lower respiratory tract (including trachea, bronchi and lungs). The inflammation in the upper and lower respiratory tract may be associated with or caused by viral infection or an allergen. It is expected that the anti-inflammatory activity of the compounds either alone or when co-administered with a glucocorticoid would make them particularly suitable for treatment of these disease or conditions.

A symptom of respiratory disease may include cough, excess sputum production, a sense of breathlessness or chest tightness with audible wheeze. Exercise capacity may be quite limited. In asthma the FEV1.0 (forced expiratory volume in one second) as a percentage of that predicted nomographically based on weight, height and age, may be decreased as may the peak expiratory flow rate in a forced expiration. In COPD the FEV1.0 as a ratio of the FVC is typically reduced to less than 0.7. The impact of each of these conditions may also be measured by days of lost work/school, disturbed sleep, requirement for bronchodilator drugs, requirement for glucocorticoids including oral glucocorticoids.

The existence of, improvement in, treatment of or prevention of a respiratory disease may be determined by any clinically or biochemically relevant method of the subject or a biopsy therefrom. For example, a parameter measured may be the presence or degree of lung function, signs and symptoms of obstruction; exercise tolerance; nighttime awakenings; days lost to school or work; bronchodilator usage; Inhaled corticosteroid (ICS) dose; oral glucocorticoid (GC) usage; need for other medications; need for medical treatment; hospital admission.

As used herein, the term respiratory infection means an infection by virus or bacteria anywhere in the respiratory tract. Examples of respiratory infection include but are not limited to colds, sinusitis, throat infection, tonsillitis, laryngitis, bronchitis, pneumonia or bronchiolitis. Preferably, in any embodiment of the invention the respiratory infection is a cold.

An individual may be identified as having a respiratory tract infection by viral testing and may exhibit symptoms of itchy watery eyes, nasal discharge, nasal congestion, sneezing, sore throat, cough, headache, fever, malaise, fatigue and weakness. In one aspect, a subject having a respiratory infection may not have any other respiratory condition. Detection of the presence or amount of virus may be by PCR/sequencing of RNA isolated from clinical samples (nasal wash, sputum, BAL) or serology.

The compounds of the invention demonstrate improved solution stability under accelerated degradation conditions relative to other related compounds. Solution stability may be assess by measuring the concentration of compound in a solution at day 0 and comparing the concentration of the compound after a period of time, such as 14 days. Solution stability may be assessed under ambient conditions, eg 25° C. and 65% relative humidity, or under accelerated conditions, eg 40° C. and 75% relative humidity. Typically, an acceptable stability for a compound of interest for the indications of the invention when stored for 14 days in solution under accelerated conditions would be retention of at least 80% concentration in the solution of the compound relative to the initial concentration of the compound in the solution. Typically, the solution may be a saline solution (eg 0.9% aq. NaCl) or phosphate-buffered saline (PBS; eg pH 7.4). In some embodiments, the compounds of the invention after 14 day storage in pH 7.4 PBS buffer is at least about 80%, 85%, 90%, 91%, 92% or greater relative to the amount of compound detected in the solution at day 0.

The compounds of the invention as described herein or a pharmaceutically acceptable salt, solvate or prodrug thereof may be covalent irreversible or covalent reversible agonists of the active site of a protein.

Where a protecting group (PG) is referred to, a person skilled in the art would readily understand what type of protecting group would be suitable. Examples of suitable amine protecting groups for the purposes described herein include (but are not limited to) tert-butyloxycarbonyl (t-Boc) and 9H-fluoren-9-ylmethoxycarbonyl (Fmoc).

Pharmaceutical compositions may be formulated from compounds of the invention as described herein for any appropriate route of administration including, for example, topical (for example, transdermal or ocular), oral, buccal, respiratory (for example, nasal, inhalation, intrapulmonary), vaginal, rectal or parenteral administration. The term parenteral as used herein includes subcutaneous, intradermal, intravascular (for example, intravenous), intramuscular, spinal, intracranial, intrathecal, intraocular, periocular, intraorbital, intrasynovial and intraperitoneal injection, as well as any similar injection or infusion technique. Suitable oral forms include, for example, tablets, troches, lozenges, aqueous or oily suspensions, dispersible powders or granules, emulsions, hard or soft capsules, or syrups or elixirs. For intravenous, intramuscular, subcutaneous, or intraperitoneal administration, one or more compounds may be combined with a sterile aqueous solution which is preferably isotonic with the blood of the recipient. Such formulations may be prepared by dissolving solid active ingredient in water containing physiologically compatible substances such as sodium chloride or glycine, and having a buffered pH compatible with physiological conditions to produce an aqueous solution, and rendering said solution sterile. The formulations may be present in unit or multi-dose containers such as sealed ampoules or vials. Examples of components are described in Martindale—The Extra Pharmacopoeia (Pharmaceutical Press, London 1993) and Martin (ed.), Remington's Pharmaceutical Sciences. Preferably, the compositions are formulated for administration to the respiratory tract, for example, by intrapulmonary administration (eg. inhalation) or intranasal administration. The compositions may be administered to the upper and/or lower respiratory tract.

Preferably, the pharmaceutical compositions are in a form suitable for administration via the respiratory route, and may be in any form such as a powder, liquid or suspension. Such compositions may target tissue including pulmonary tissue (including alveolus, terminal bronchiole, bronchiole, and bronchus) or the nasal cavity (including paranasal cavity, frontal sinus, ethmoid sinus, maxillary sinus, sphenoidal sinus, superior turbinate, middle turbinate, and inferior turbinate).

In the context of this specification the term “administering” and variations of that term including “administer” and “administration”, includes contacting, applying, delivering or providing a compound or composition of the invention to an organism, or a surface by any appropriate means.

The dose of the biologically active compound according to the invention may vary within wide limits and may be adjusted to individual requirements. Active compounds according to the present invention are generally administered in a therapeutically effective amount.

A composition according to the present invention is to be administered in an effective amount. The phrase ‘therapeutically effective amount’ or ‘effective amount’ generally refers to an amount of a compound of the invention described herein, a pharmaceutically acceptable salt, polymorph or prodrug thereof of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g. side effects, are sometimes manifested along with the desired therapeutic effect; hence, a practitioner balances the potential benefits against the potential risks in determining what is an appropriate “effective amount”.

The exact amount required will vary from subject to subject, depending on the species, age and general condition of the subject, mode of administration and the like. However, an appropriate “effective amount” in any individual case may be determined by one of ordinary skill in the art using only routine experimentation. In one aspect, the dose administered to a subject is any dose that reduces viral load. Preferably, the dose does not significantly increase inflammation, for example does not significantly increase absolute neutrophil numbers or the proportion of neutrophils of total BAL cells in the lung. The terms “therapeutically effective amount” or “effective amount” may also refer to an amount of the compound of the invention or a pharmaceutically acceptable salt, solvate or prodrug thereof, which results in an improvement or remediation of the symptoms of a respiratory infection, or respiratory disease or condition associated with a viral or bacterial infection.

In some embodiments, an effective amount for a human subject lies in the range of about 500 nmoles/dose to about 0.005 nmoles/dose, or about 250 nmoles/dose to 0.005 nmoles/dose. Preferably, the range is about 250 nmoles/dose to 0.05 nmoles/dose. In some embodiments, the dose range is about 500 nmoles to about 0.1 nmoles, about 250 nmoles to 0.1 nmoles, about 50 nmoles to 0.1 nmoles, about 5 nmoles to 0.1 nmol, about 2.5 nmoles to 0.25 nmoles, or about 0.5 nmoles to 0.1 nmoles. In some embodiments, the amount is at, or about, 500 nmoles, 250 nmoles, 50 nmoles, 5 nmoles, 2.5 nmoles, 0.5 nmoles, 0.25 nmoles, 0.1 nmoles or 0.05 nmoles of the compound. Dosage regimes are adjusted to suit the exigencies of the situation and may be adjusted to produce the optimum therapeutic dose.

In some embodiments, an effective amount for a single dose for a human subject may be in the range of about 0.1 μg to about 1000 μg. Preferably, the effective amount for a single dose may be from about 1 μg to about 1000 μg. In some embodiments, the effective amount may be from about 1 μg to about 1000 μg, about 1 μg to about 500 μg, about 1 μg to about 250 μg, about 1 μg to about 100 μg, about 1 μg to about 50 μg, about 1 μg to about 45 μg, about 1 μg to about 40 μg, about 1 μg to about 30 μg, about 5 μg to about 30 μg, about 10 μg to about 30 μg, about 5 μg to about 25 μg, about 10 μg to about 25 μg, about 15 μg to about 25 μg. In some embodiments, the effective amount for a single dose for a human subject may be at, or about 20 μg. In some embodiments, the dosage may be titrated, with the initial dose being within the dosage ranges described herein.

The dosage may be delivered through administration of a unit dosage form containing the entire effective amount of the active compound. Alternatively, the dosage may be delivered by administering a number of discrete unit dosage forms, which collectively contain the effective amount of the active compound.

The pharmaceutical compositions of the invention may be suitable for administration via inhalation, and therefore may be provided in a suitable form, including dry powder, sprays, mists, or aerosols. This may be particularly preferred for treatment of a respiratory infection. For inhalation formulations, the composition or combination provided herein may be delivered via any inhalation methods known to a person skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient. Alternatively, the composition may be a dry powder and administered to the respiratory tract as defined herein.

It will be understood, that the specific dose level for any particular patient will depend upon a variety of factors including the activity of the specific compound employed, the age, body weight, general health, sex, diet, time of administration, route of administration, and rate of excretion, drug combination (i.e. other drugs being used to treat the patient), and the severity of the particular disorder undergoing therapy.

It will be understood, however, that the specific dose level for any particular subject will depend upon a variety of factors including the activity of the specific compound employed, the age, body weight, general health, sex, diet, time of administration, route of administration, and rate of excretion, drug combination (i.e. other drugs being used to treat the subject), and the severity of the particular disorder undergoing therapy. The dosage will generally be lower if the compounds are administered locally rather than systemically, and for prevention rather than for treatment. Such treatments may be administered as often as necessary and for the period of time judged necessary by the treating physician. A person skilled in the art will appreciate that the dosage regime or therapeutically effective amount of the compound of the invention, or a pharmaceutically acceptable salt, solvate or prodrug thereof, to be administered may need to be optimized for each individual. The pharmaceutical compositions may contain active ingredient in the range of about 0.01 to 2000 mg, preferably in the range of about 0.05 to 500 mg and most preferably between about 0.01 and 200 mg or 0.01 and 100 mg. A daily dose of about 0.01 to 100 mg, preferably between about 0.1 and about 50 mg, may be appropriate. The daily dose can be administered in a single or multiple doses per day.

It will also be appreciated that different dosages may be required for treating different disorders.

As used herein, the terms “treatment” or “treating” of a subject includes the application or administration of a compound or composition of the invention to a subject (or application or administration of a compound of the invention to a cell or tissue from a subject) with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

As used herein, “preventing” or “prevention” is intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). Biological and physiological parameters for identifying such patients are provided herein and are also well known by physicians.

“Subject” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs.

### Spray Drying

In another aspect, there is provided a method of preparing a powder comprising a compound comprising a TLR2 agonist moiety conjugated with a solubilising moiety, or a pharmaceutical acceptable salt, solvate, stereoisomer or prodrug thereof, and a cyclodextrin, the method comprising:
forming a solution comprising the compound and the cyclodextrin; and
spray drying the solution to provide the powder.

Spray drying is reviewed in Alhajj, N. et al. Powder Technology 384 (2021) 313-331, which is entirely incorporated herein by reference. Any suitable spray drying methodology may be employed.

Any suitable spray dryer may be used in these methods. In some embodiments, the spray dryer comprises an ultrasonic nozzle or a pressure swirl nozzle. Accordingly, the spray drying step of the methods described herein comprises spraying the solution through a nozzle.

Parameters that can be adjusted to control the spray drying process include the outlet temperature of the solution as it exits the nozzle and the flow rate of the solution fed into the nozzle. In the methods described herein, any suitable outlet temperature and flow rate may be employed.

In embodiments, the outlet temperature may be from about 40° C. to about 90° C., about 65° C. to about 85° C. or about 70° C. to about 80° C. As the liquid carrier is the component removed from the solution during the spray drying step, the selection of liquid carrier may influence the target outlet temperature. The means of achieving the outlet temperature may vary depending on the nozzle-type selected, for example an ultrasonic nozzle may control the outlet temperature depending on the frequency selected for the ultrasonic wave.

In embodiments, the flow rate of the solution fed into the nozzle may be from about 0.1 g/min (gram per minute) to about 10 g/min, about 1 g/min to about 5 g/min or about 1.5 g/min to about 2.0 g/min.

Preferably, the parameters of the spray drier are adjusted to provide a powder possessing <10% of particles below 10 μm. The particle size may be determined by any suitable method, preferably by laser particle size analysis.

The powder produced by these methods may be amorphous or crystalline. The crystallinity of the powder may depend on the particular combination of compound and excipients included.

The powder is produced by removing liquid components from the solution. The powder may be any powder form of a pharmaceutical composition described herein. Accordingly, the solution may comprise any pharmaceutical composition described herein and a liquid carrier. Any liquid carrier compatible with the ingredients included in the pharmaceutical composition and removable under spray drying conditions may be used. Typically the liquid carrier comprises one or more polar solvents. Suitable polar solvents include water, acetonitrile (ACN), dimethylsulfoxide (DMSO), methanol (MeOH), ethanol (EtOH) and combinations thereof. In the solutions to be spray dried, the liquid carrier does not need to be pharmaceutically acceptable provided it is able to be substantially removed during the spray drying process. However, preferably the liquid carrier is also pharmaceutically acceptable in case residual carrier remains in the powder.

The solution may comprise the compound in a minimum concentration of at least about 0.1 wt %, 0.5 wt % or 1 wt %. The solution may comprise the compound in a maximum concentration of not more than about 20 wt %, 15 wt %, 10 wt % or 5 wt %. The solution may comprise the compound from any of these minimum amounts to any maximum amount, for example, from about 0.1 wt % to about 20 wt % or about 0.5 wt % to about 10 wt %. The concentration of compound (and any excipient present, including cyclodextrin) will be lower in the solution than in the powder produced by the process.

In some embodiments, the method of preparing a powder further comprises a step of combining the powder produced by spray drying the solution with one or more further excipients. The further excipient may be any pharmaceutically acceptable excipient in a form suitable for the desired route of administration for the powder. For example for administration by inhalation, the further excipient will preferably possess suitable particle size properties. In some embodiments, the further excipient may be a sugar compound, preferably mannitol.

Another aspect provides a solution comprising a pharmaceutical composition and a liquid carrier. The solution may be any of the solutions capable of being spray dried to provide a powdered form of a pharmaceutical composition of the invention described herein.

### Specific Embodiments

In some embodiments, the pharmaceutical composition comprises the compound comprising a TLR2 agonist moiety conjugated with a solubilizing moiety, a cyclodextrin and one or more further excipients. The one or more further excipients preferably include a sugar compound, such as mannitol, and optionally leucine. In these embodiments, the pharmaceutical composition may be a solid form, such as a powder. The powder may be provided by spray drying a solution comprising all of the components of the pharmaceutical composition and a liquid carrier, wherein the liquid carrier is substantially removed during the spray drying process. The spray dried powder is preferably adapted for administration by inhalation and may have any of the particle size properties described herein (eg X₅₀ values). The particles of the powder form may preferably possess an X₅₀ of about 20-50 μm (more preferably about 20-30 μm) with <10% of particles below 10 μm.

In some embodiments, the pharmaceutical composition in powder form, comprises:
a compound comprising a TLR2 agonist moiety conjugated with a solubilizing moiety as described herein;
a cyclodextrin;
a sugar compound selected from mannitol, myo-inositol and any combination thereof; and
optionally one or more further excipients.

In some embodiments, the pharmaceutical composition in powder form comprises:
about 0.5 wt % to about 5 wt % of a compound comprising a TLR2 agonist moiety conjugated with a solubilizing moiety;
about 5 wt % to about 94.5 wt % of a cyclodextrin
about 5 wt % to about 94.5 wt % sugar compound, wherein the sugar compound is selected from mannitol, myo-inositol and a combination thereof; and
about 0 wt % to about 20 wt % one or more further excipients.

In some embodiments, the pharmaceutical composition in powder form comprises:
about 1 wt % to about 5 wt % of a compound comprising a TLR2 agonist moiety conjugated with a solubilizing moiety;
about 5 wt % to about 94 wt % of a cyclodextrin
about 5 wt % to about 94 wt % sugar compound, wherein the sugar compound is selected from mannitol, myo-inositol and a combination thereof; and
about 0 wt % to about 20 wt % one or more further excipients.

In some embodiments, the pharmaceutical composition in powder form comprises:
about 1 wt % to about 5 wt % of a compound comprising a TLR2 agonist moiety conjugated with a solubilizing moiety;
about 30 wt % to about 84 wt % of a cyclodextrin
about 15 wt % to about 69 wt % sugar compound, wherein the sugar compound is selected from mannitol, myo-inositol and a combination thereof; and
about 0 wt % to about 20 wt % one or more further excipients.

In some embodiments, the pharmaceutical composition in powder form comprises:
about 0.5 wt % to about 5 wt % of a compound comprising a TLR2 agonist moiety conjugated with a solubilizing moiety;
about 5 wt % to about 93.5 wt % of a cyclodextrin
about 5 wt % to about 93.5 wt % sugar compound, wherein the sugar compound is selected from mannitol, myo-inositol and a combination thereof; and
about 1 wt % to about 20 wt % one or more further excipients.

In some embodiments, the pharmaceutical composition in powder form comprises:
about 1 wt % to about 5 wt % of a compound comprising a TLR2 agonist moiety conjugated with a solubilizing moiety;
about 30 wt % to about 84 wt % of a cyclodextrin
about 15 wt % to about 69 wt % sugar compound, wherein the sugar compound is selected from mannitol, myo-inositol and a combination thereof; and
about 0 wt % to about 20 wt % one or more further excipients, wherein the powder comprises, or consists of, spray dried particles with an X₅₀ of about 20-30 μm and wherein <10% of the spray dried particles have a particle size below 10 μm. The particle size may be determined by any means described herein.

In embodiments comprising both cyclodextrin and mannitol, the ratio of cyclodextrin to mannitol may be from about 1:1 to about 2:1.

In embodiments, the cyclodextrin is selected from β-cyclodextrin, hydroxypropyl-β-cyclodextrin or a combination thereof. In embodiments, the cyclodextrin is β-cyclodextrin. In embodiments, the cyclodextrin is hydroxypropyl-β-cyclodextrin.

In embodiments, the sugar compound is mannitol.

In embodiments, the one or more further excipients are selected from a surface modification agent and a polymer (preferably a viscosity modifying agent) or a combination thereof. In embodiments, the one or more further excipients consist of a viscosity modifying agent, such as any of the viscosity modifying agents described herein, including polymers.

In some embodiments, the pharmaceutical compositions are substantially free of polymer. In embodiments, the pharmaceutical compositions are substantially free of hydroxypropylmethyl cellulose and/or microcrystaline cellulose.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1—Synthesis of Compounds

### Example 1.1—Synthesis a Using Fmoc Solid Phase Chemistry

The synthesis of compounds of formula (I) are described in WO 2019/119067 (US 2021/0230217 A1) and WO 2020/257870 (U.S. application Ser. No. 17/622,451). This example describes the synthesis of compounds of formula (XX) (also described in WO 2021/258154). The syntheses of all of these compounds follow a similar synthetic strategy and the skilled person will be able to make the necessary variations to prepare the desired compound(s) of the invention based on similar techniques or by other protocols known in the art.

Compounds of formula (XX) may be provided by coupling a compound of the formula A-1:

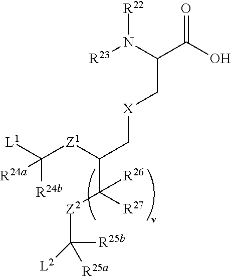

wherein R²¹, R²², R^{24a}, R^{24b}, R^{25a}, R^{25b}, R²⁶, R²⁷, X, Z¹, Z², v, L¹ and L² have the meanings as defined for any compound of the invention defined herein and R²² is an amino protecting group
with a compound of formula YB-I:

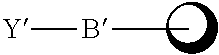

wherein
Y′ is

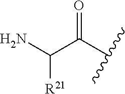

wherein R²¹ is selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
B′ is a radical of PEG as defined for any compound of the invention; and
[symbol] is a solid support resin.

In some embodiments, B′ comprises a substituted PEG of Formula B-I. In these embodiments, the following sequence of solid phase reactions may be employed:
a) Optionally coupling 1 to 10 alpha amino acids or compounds derived from a natural alpha amino acid, that constitutes L, to a solid phase resin using Fmoc chemistry
b) Coupling PG-NH—(CH₂)ₚ—O—(CH₂CH₂O)ₙ—(CH₂)ₘ—COOH to a solid phase resin or substituted resin if L is present, wherein PG represents an amino protecting group compatible with Fmoc chemistry;
c) Removing PG;
d) Coupling PG-NH—CR₁₃R₁₄—COOH, wherein PG′ represents an amino protecting group compatible with Fmoc chemistry;
e) Removing PG′;
f) Coupling an acid of the formula (A-1);
g) Optionally removing R²² and optionally acylating and/or alkylating to introduce R²² and/or R²²; and
h) Removing the compound from the solid phase support

In some embodiments, B′ comprises a substituted PEG according to formula (B-II) and the following sequence of solid phase reactions may be employed:
a) Optionally coupling 1 to 10 alpha amino acids or compounds derived from a natural alpha amino acid, that constitute L, to a solid phase resin using Fmoc chemistry
b) Coupling PG-NH—(CH₂)ₜ—O—(CH₂CH₂O)ₖ—(CH₂)ₕ-COOH to a solid phase resin or substituted resin if L is present, wherein PG represents an amino protecting group compatible with Fmoc chemistry;
c) Removing PG;
d) Coupling PG′-NH—(CH₂)ₚ—O—(CH₂CH₂O)ₙ—(CH₂)ₘ—COOH, wherein PG′ represents an amino protecting group compatible with Fmoc chemistry;
e) Removing PG′;
f) Coupling PG″-NH—CR₁₃R₁₄—COOH, wherein PG″ represents an amino protecting group compatible with Fmoc chemistry;
g) Removing PG″;
h) Coupling an acid of the formula (A-1);
i) Optionally removing R²² and optionally acylating and/or alkylating to incorporate R²² and/or R²²; and
j) Removing the compound from the solid phase resin.

It will be appreciated that the exact sequence of events can be varied from that outlined, and additional steps added where necessary and synthetically expedient, for example oxidation of the cysteine sulfur to the sulfoxide.

### Example 1.2—Synthesis of Intermediate for Use in the Solid Phase Coupling A

Some embodiments of the intermediate acid of formula A-II:

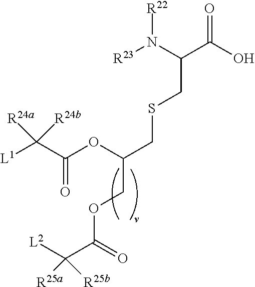

wherein R²², R²³, R^{24a}, R^{24b}, R^{25a}, R^{25b}, L¹, L² and v are as defined for the compound of formula A-I above; may be prepared by the synthesis shown in Scheme 1.

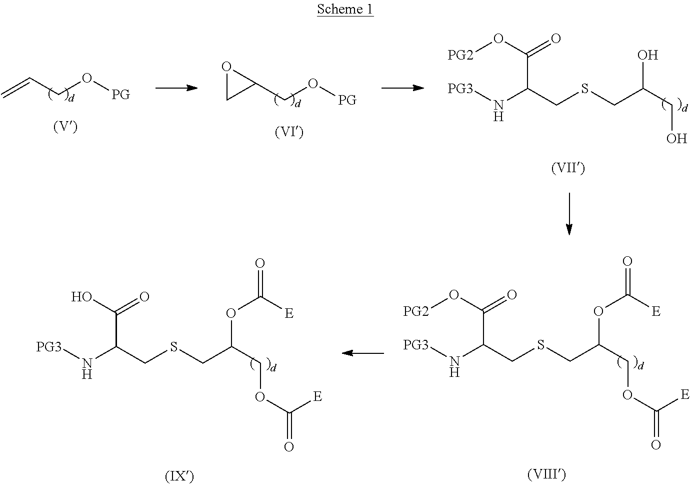

In scheme 1, PG represents an alcohol protecting group, PG2 represents an carboxylic acid protecting group, PG3 represents an amide protecting group (and corresponds to R²³) and E represents:

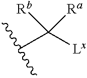

wherein L^{x} is as defined for L¹ and L², R^{a} is as defined for R^{24a} and R^{25a} and R^{b} is as defined for R^{24b} and R^{25b}.

Reaction of protected alkene alcohols of the formula (V′), where PG is a suitable protecting group, for example a silyl group such as TBDMS, forms an epoxide of the formula (VI′). It will be appreciated that the epoxide formation may be carried out to give the product racemically or to give enantioenriched material. If a racemic or scalemic mixture of enantiomers is produced preparative chiral chromatography is employed to separate the enantiomers if required.

Epoxides of the formula (VI′) are reacted with suitably protected cystine analogues, for example tert-butyl N-(((9H-fluoren-9-yl)methoxy)carbonyl)-S—(((R)-2-((((9H-fluoren-9-yl)methoxy)carbonyl)amino)-3-(tert-butoxy)-3-oxopropyl)thio)-D-cysteinate, where PG2 is a tert-butyl ester and PG3 is Fmoc, under reducing conditions to give alcohols of the formula (VII′). It will be appreciated that alcohols of the formula (VII′) can be comprised of more than one stereoisomer and where stereoisomers are present these can be separated by chiral preparative chromatography as required.

Alcohols of the formula (VII′) can be acylated to give carbonyl containing adducts of the formula (VIII′) using suitable reagents, for example with a suitably substituted acid chloride reacted in the presence of suitable bases and solvents. Carbonyl containing adducts of the formula (VIII′) can then be deprotected to reveal carboxylic acids of the formula (IX′) using suitable reagents, for example where PG2 is tert-butyl, trifluoroacetic acid can be used to preferentially remove the tert-butyl group.

Acids of the formula (IX′) can then be used as reagents in solid phase synthesis such as those described herein.

### Example 1.3—Synthesis B Using Fmoc Solid Phase Chemistry

Compounds of the invention, including those according to Formula (I) may be provided by preparing a resin bound peptide of the following formula:

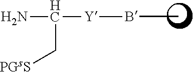

wherein
Y′ is

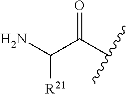

wherein R²¹ is selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
B′ is a Polyethylene Glycol (PEG);
PG^{s} is H or a sulphur protecting group, such as tert-butyl; and
◯ is a solid support resin.

Following optional sulphur deprotection, this resin bound peptide may be reacted with a 1,2-epoxy-alkanol of the following formula:

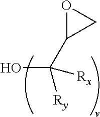

wherein Rₓ, R_{y} and v have the meanings given for Formula (I)
to provide an alkylated thiol of formula S—1:

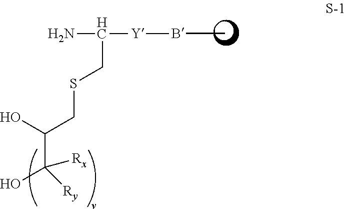

wherein Y′ and B′ have the meaning given above, and v has the meaning given for the compound of formula (I), or a sulfone or sulfoxide thereof.

The diol moieties of resin bound compound S—1 may be further reacted with an appropriately substituted carboxylic acid (typically in an activated form such as acid chloride, mixed anhydride, etc. or in the presence of a coupling reagent, such as diisopropylcarbodiimide (DIC), dicyclohexyl carbodiimide (DCC), Hydroxybenzotiazole (HOBt, available as Oxyma Pure), etc., optionally together with a catalyst such as 4-dimethylaminopyridine (DMAP)) to provide a compound of the invention.

### Example 1.4—Synthesis and Characterisation of Compounds B4, B6, B8, B12, B14 and B16

Synthesis of compound B12. Compound B12 was synthesized by standard Fmoc Solid Phase Peptide Synthesis, starting with Fmoc-RINK MBHA PS Resin. Removal of the Fmoc group after each coupling was achieved using 20% Piperidine in DMF. Couplings of Fmoc-Gly-OH (2-fold excess), Fmoc-NH-PEG₂₈-CH₂CH₂COOH (1.4-fold excess), Fmoc-Ser(tBu)-OH (2-fold excess), and N-(Boc)-S—((R)-2,3-dihydroxybutyl)-L-cysteine (1.5-fold excess) were performed in diemethyl formamide (DMF) using equivalent excess of Oxyma Pure and DIC as coupling agents. 2-Methyl-Palmitic Acid coupling was performed using 2-Methyl-Palmitic Acid (20 eq. vs. moles resin), DIC (20 eq.), DMAP (2 eq.) in dichloromethane (DCM)/tetrahydrofuran (THF) (85/15) (v/v) for ˜20 hours at room temperature (˜25° C.). Optionally following each coupling step any unreacted peptide fragments are capped with an acetyl group by reaction with acetic anhydride, which may assist in purification of the compound from deletion products.

Cleavage of the peptide from the resin, removal of N-terminal Boc group, and serine side-chain deprotection were achieved by exposure of the resin to a solution of 93% TFA, 5% H₂O, 3% TIPS for 1.5 hours. Following the cleavage reaction, the mixture was evaporated and the resulting residue was re-dissolved in 30% Acetonitrile/Water and lyophilized.

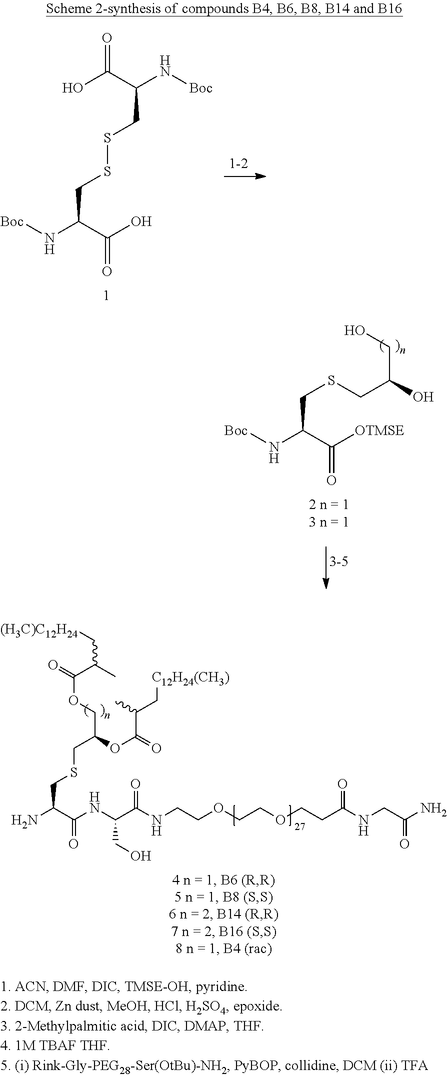

### Procedures for Synthetic Steps of Scheme 2

### Synthesis of Rink-Gly-PEG28-Ser(OtBu)-NH₂

1. β-[(R,S)-α-[1-(9H-Fluoren-9-yl)-methoxyformamido]-2,4-dimethoxybenzyl]-phenoxyacetic (Fmoc-Rink) amide AM resin 0.47 meq/g, 5 g 2.35 mmol). The resin was swollen in dimethyl formamide (DMF; 30 mL) for 15 minutes (min) and then the solvent filtered off.

2. The resin was then treated with 20 mL of 20% piperidine/DMF twice (1×5 min and then 1×10 min)

3. The resin was washed with DMF×2 and then DCM×2. Bromophenol blue (BPB) test positive

4. Fmoc-Gly-OH (3 equivalents (eq), 7.05 mmol, 2.1 g) in DMF 15 mL was added PyBOP (7.05 mmol, 3.67 g) and then diisopropylethyl amine (DIPEA; 4 eq, 9.4 mmol, 1.64 mL) and mixed and left to stand for 5-10 min.

5. The mixture was added to the resin and the resin/mixture was shaken for 2 hours (h). After 2 h BPB test was negative.

6. The resin was filtered and then washed with DMF×2 and then dichloromethane (DCM)×2 and finally DMF.

7. The resin was then treated with 20 mL of 20% piperidine/DMF twice (1×5 min and then 1×10 min)

8. The resin was washed with DMF×2 and then DCM×2. BPB test positive

9. 200 mg of the NH₂-Gly-Rink-resin (assume 0.47 meq/g, 0.094 mmol) was swollen in DCM, then filtered and washed several times with DCM.

10. Fmoc-NH-PEG28—(CH2)2-CO2H (MW=1544.75, 1.5 eq, 0.141 mmol 145 mg) was taken up in 4 mL DCM and PyBOP (1.6 eq, 0.150 mmol 78 mg) was added followed by DIPEA (4 eq, 0.376 mmol 0.065 mL) and stirred for several minutes then added to the resin, which was shaken overnight.

11. BPB test was negative. The resin was washed with DMF×2 and then DCM×2 and finally DMF.

12. The resin was then treated with 1 mL of 20% piperidine/DMF twice (1×5 min and then 1×10 min).

13. The resin was washed with DMF×2 and then DCM×2. BPB test positive

14. Fmoc-Ser(OtBu)OH (MW=383.4, 1.5 eq, 0.141 mmol 54 mg) was taken up in 4 mL DMF and PyBOP (1.6 eq, 0.150 mmol 78 mg) was added followed by DIPEA (4 eq, 0.376 mmol 0.065 mL) and stirred for several minutes then added to the resin, which was shaken overnight.

15. BPB test was negative. The resin was washed with DMF×2 and then DCM×2 and finally DMF.

16. The resin was then treated with 1 mL of 20% piperidine/DMF twice (1×5 min and then 1×10 min).

17. The resin was washed with DMF×2 and then DCM×2. BPB test positive.

### Step 1

N′N″-Bis-Boc-L-cystine 1 (6.61 g, 15 mmol) was dissolved in ACN (30 mL) and DMF (11.25 mL) in a two neck 100 mL flask in an N2 atm and chilled on an ice bath. To this mixture was added 2-(trimethylsilyl)ethanol (5.11 mL, 35.63 mmol) and pyridine (4.80 mL, 59.33 mmol) and left to stir on an ice bath for ten minutes then DCC (6.75 g, 32.70 mmol) was added and stirred on an ice bath for 16h without recharging the ice bath. To the mixture was added solid citric acid and stirred a further 1-2h. The mixture was diluted with ether and filtered through a silica plug. The organic layer was washed with 5% citric acid, water, bicarbonate then brine, dried and concentrated to a clear resin that was used without further purification 10.3 g 1H NMR (401 MHz, CDCl3) δ 5.38 (d, J=7.1 Hz, 1H), 4.56 (d, J=7.1 Hz, 1H), 4.33-4.16 (m, 3H), 3.16 (s, 2H), 1.46 (d, J=5.2 Hz, 14H), 1.03 (dd, J=9.0, 8.4 Hz, 3H), 0.11-0.02 (m, 13H).

### Step 2

The product of step 1 (10.3 g, 15 mmol based on intermediate 1 reaction) was taken up in DCM (80 mL) and Zn dust (8.12 g, 124.15 mmol) was added. The mixture was cooled on an ice bath. To the mixture was added freshly prepared methanol (MeOH):cHCl:cH₂SO₄ (100:7:1) (32 mL) and stirred on ice for 0.5h and then the ice bath was removed, (R)-(+)-oxirane-2-methanol added and stirred at 40° C. overnight. The mixture was cooled to room temperature (rt), diluted with dichloromethane (DCM) and filtered through celite. The organic phase was washed with water and then brine. The combined aqueous phases were back extracted with diethyl ether. The combined organics were dried, filtered and concentrated to provide intermediate compound 2 as a clear colourless resin 11.52 g 98% yield and used without further purification.

Intermediate compound 3 was prepared by a similar route to that followed for the preparation of intermediate compound 2, except (R)-(+)-Oxirane-2-ethanol was used instead of (R)-(+)-Oxirane-2-methanol). ¹H NMR (400 MHz, CDCl3) δ 4.62-4.36 (m, 1H), 4.32-4.18 (m, 2H), 3.97-3.79 (m, 3H), 3.02 (dt, J=18.4, 9.2 Hz, 1H), 2.94-2.70 (m, 2H), 2.65-2.46 (m, 2H), 1.85-1.66 (m, 2H), 1.45 (s, 9H), 1.12-0.95 (m, 2H), 0.07-0.02 (m, 9H).

### Steps 3-5

Synthesis of compound B6 (R, R). A portion of intermediate compound 2 (100 mg, 0.253 mmol) was taken up in anhydrous THE (1.5 mL) in an N₂ atmosphere and to this mixture was added (R)-2-methylpalmitic acid (205 mg, 0.758 mmol), N-dimethylaminopyridine (DMAP; 12 mg, 0.101 mmol) and finally diisopropyl carbodiimide (DIC; 118 μL, 0.758 mmol) at rt in an N2 atmosphere and stirred at rt overnight. Then diluted in ether and filtered. The organic layer was washed with 1M HCl, water, bicarbonate, water then brine, dried (MgSO₄), filtered and concentrated to a residue that solidifies to a waxy solid. The crude material was taken up in 1M tert-butylammonium fluroride complex with tetrahydrofuran (TBAF THF; 1.4 mL) and stirred at rt for 3 h. The mixture was diluted in ether and washed with 1M HCl, then water×4 and finally brine. The organic layer was dried (MgSO₄), filtered and concentrated to a residue (89 mg). The crude material was taken up in DCM (1 mL) and benzotriazol-1-yl-oxytripyrrolidinophosphonium hexafluorophosphate (PyBOP) added (65 mg), followed by collidine (27 μL) stirred for 1 min, then added to Rink-Gly-PEG28-Ser(OtBu)-NH₂ resin (100 mg) in DCM (2 mL). After 2h BPB test was negative. The resin was thoroughly washed with DMF×2 and then DCM×4, MeOH and then diethyl ether×4. Then left under high vacuum overnight. The resin was treated with 95% trifluoroacetic acid (TFA) 5% tiisopropylsilane (TIPS) for 2 h. Then filtered and TFA removed under N₂ flow. The remaining residue was taken up in water and freeze dried. The lyophilised material was purified on by high-performance liquid chromatography (HPLC) isocratic flow 80:20 A:B (A=50:50 acetonitrile (ACN):MeOH, B=1% A in water) to provide after lyophilisation 35.2 mg of amorphous solid 6.5% yield. LCMS R_{f} (min)=6.73. MS m/z 1074.4.0 (M+2H)/2, 722.4 (M+3H+H2O)/3. HR-ESI calcd for C₁₀₄H₂₀₃N₅O₃₇S (M+2H)/2, 1074.7028. found, 1074.7030.

Synthesis of compound B8 (S, S). A portion of intermediate compound 2 (100 mg, 0.253 mmol) was taken up in anhydrous THE (1.5 mL) in an N2 atmosphere and to this mixture was added (S)-2-methylpalmitic (205 mg, 0.758 mmol), DMAP (12 mg, 0.101 mmol) and finally DIC (118 μL, 0.758 mmol) at rt in an N2 atmosphere and stirred at rt overnight. Then diluted in diethyl ether and filtered. The organic layer was washed with 1M HCl, water, sodium bicarbonate (aq.), water then brine, dried (MgSO₄), filtered and concentrated to a residue that solidifies to a waxy solid. The crude material was taken up in 1M TBAF THE (1.5 mL) and stirred at rt for 3 h. The mixture was diluted in diethyl ether and washed with 1M HCl, then water×4 and finally brine. The organic layer was dried (MgSO₄), filtered and concentrated to a residue 104 mg. The crude material was taken up in DCM (1 mL) and PyBOP added (76 mg), followed by collidine (32 μL) stirred for 1 min, then added to Rink-Gly-PEG28-Ser(OtBu)-NH₂ resin (117 mg) in DCM (2 mL). After 2h BPB test was negative. The resin was thoroughly washed with DMF×2 and then DCM×4, MeOH and then diethyl ether×4. Then left under high vacuum overnight. The resin was treated with 95% TFA 5% TIPS for 2 h. Then filtered and TFA removed under N₂ flow. The remaining residue was taken up in water and freeze dried. The lyophilised material was purified on by HPLC isocratic flow 80:20 A:B (A=50:50 ACN:MeOH, B=1% A in water) to provide after lyophilisation 44.2 mg of amorphous solid 8.1% yield. LCMS R_{f} (min)=6.64. MS m/z 1074.4.0 (M+2H)/2, 722.4 (M+3H+H2O)/3. HR-ESI calcd for C₁₀₄H₂₀₃N₅O₃₇S (M+2H)/2, 1074.7028. found, 1074.7038.

Synthesis of compound B14 (R, R). A portion of intermediate compound 3 (104 mg, 0.253 mmol) was taken in anhydrous THE (1.5 mL) in an N2 atmosphere and to this mixture was added (R)-2-methylpalmitic (205 mg, 0.758 mmol), DMAP (12 mg, 0.101 mmol) and finally DIC (118 μL, 0.758 mmol) at rt in an N₂ atmosphere and stirred at rt overnight. Then diluted in diethyl ether and filtered. The organic layer was washed with 1M HCl, water, sodium bicarbonate (aq.), water then brine, dried (MgSO₄), filtered and concentrated to a residue that solidifies to a waxy solid. The crude material was taken up in 1M TBAF THE (1.4 mL) and stirred at rt for 3 h. The mixture was diluted in diethyl ether and washed with 1M HCl, then water×4 and finally brine. The organic layer was dried (MgSO₄), filtered and concentrated to a residue (150 mg). The crude material was taken up in DCM (1 mL) and PyBOP added (110 mg), followed by collidine (46 μL) stirred for 1 min, then added to Rink-Gly-PEG28-Ser(OtBu)-NH₂ resin (168 mg) in DCM (2 mL). After 2h BPB test was negative. The resin was thoroughly washed with DMF×2 and then DCM X₄, MeOH and then diethyl ether×4. Then left under high vacuum overnight. The resin was treated with 95% TFA 5% TIPS for 2 h. Then filtered and TFA removed under N2 flow. The remaining residue was taken up in water and freeze dried. The lyophilised material was purified on by HPLC isocratic flow 80:20 A:B (A=50:50 ACN:MeOH, B=1% A in water) to provide after lyophilisation 70 mg of amorphous solid 12.8% yield. LCMS R₆ (min)=6.25. MS m/z 1081.7 (M+2H)/2, 727.1 (M+3H+H2O)/3. HR-ESI calcd for C₁₀₅H₂₀₅N₅O₃₇S (M+2H)/2, 1081.7107. found, 1081.7108.

Synthesis of compound B16 (S, S). A portion of intermediate compound 3 (104 mg, 0.253 mmol) was taken in anhydrous THE (1.5 mL) in an N2 atmosphere and to this mixture was added (S)-2-methylpalmitic (205 mg, 0.758 mmol), DMAP (12 mg, 0.101 mmol) and finally DIC (118 μL, 0.758 mmol) at rt in an N2 atmosphere and stirred at rt overnight. Then diluted in ether and filtered. The organic layer was washed with 1M HCl, water, sodium bicarbonate (aq.), water then brine, dried (MgSO₄), filtered and concentrated to a residue that solidifies to a waxy solid. The crude material was taken up in 1M TBAF THE (1.4 mL) and stirred at rt for 3 h. The mixture was diluted in diethyl ether and washed with 1M HCl, then water×4 and finally brine. The organic layer was dried (MgSO₄), filtered and concentrated to a residue (77 mg). The crude material was taken up in DCM (1 mL) and PyBOP added (56 mg), followed by collidine (23 μL) stirred for 1 min, then added to Rink-Gly-PEG28-Ser(OtBu)-NH₂ resin (87 mg) in DCM (2 mL). After 2h BPB test was negative. The resin was thoroughly washed with DMF×2 and then DCM×4, MeOH and then diethyl ether×4. Then left under high vacuum overnight. The resin was treated with 95% TFA 5% TIPS for 2 h. Then filtered and TFA removed under N2 flow. The remaining residue was taken up in water and freeze dried. The lyophilised material was purified on by HPLC isocratic flow 80:20 A:B (A=50:50 ACN:MeOH, B=1% A in water) to provide after lyophilisation 47.7 mg of amorphous solid 8.7% yield. LCMS R₆ (min)=6.35. MS m/z 1081.7 (M+2H)/2, 727.1 (M+3H+H2O)/3. HR-ESI calcd for C₁₀₅H₂₀₅N₅O₃₇S (M+2H)/2, 1081.7107. found, 1081.7132.

Synthesis of compound B4. A portion of intermediate compound 2 (100 mg, 0.253 mmol) shown in scheme 2 was taken in anhydrous THE (1.5 mL) in an N₂ atmosphere and to this mixture was added 2-methylpalmitic acid (205 mg, 0.758 mmol), DMAP (12 mg, 0.101 mmol) and finally DIC (118 μL, 0.758 mmol) at rt in an N2 atm and stirred at rt overnight. Then diluted in ether and filtered. The organic layer was washed with 1M HCl, water, bicarbonate, water then brine, dried (MgSO₄), filtered and concentrated to a residue that solidifies to a waxy solid. The crude material was taken up in 1M TBAF THE (1.5 mL) and stirred at rt for 3 hr. The mixture was diluted in ether and washed with 1M HCl, then water×4 and finally brine. The organic layer was dried (MgSO₄), filtered and concentrated to a residue (75 mg). The crude material was taken up in DCM (1 mL) and PyBOP added (55 mg), followed by collidine (23 μL) stirred for 1 min, then added to Rink-Gly-PEG28-Ser(OtBu)-NH₂ resin (84 mg) in DCM (2 mL). After 2h BPB test was negative. The resin was thoroughly washed with DMF×2 and then DCM×4, MeOH and then Ether×4. Then left under high vac overnight. The resin was treated with 95% TFA 5% TIPS for 2 h. Then filtered and TFA removed under N2 flow. The remaining residue was taken up in water and freeze dried. The lyophilised material was purified on by HPLC isocratic flow 80:20 A:B (A=50:50 ACN:MeOH, B=1% A in water) to provide after lyophilisation 9.2 mg of amorphous solid 1.7% yield. HR-ESI calc'd for C₁₀₄H₂₀₃N₅O₃₇S (M+2H)/2, 1074.7028. found, 1074.7058.

### Purification and Characterisation

Purification and characterisation: Following cleavage from the solid support, each of the analogs were purified by reversed-phase HPLC conducted using a Novasep Axial Compression Column (5-cm diameter) loaded with cyano media (Daisogel SP-120-CN-P), with a gradient of Acetonitrile in [0.1% TFA/Water]. Following intermediate lyophilization, ion-exchange was performed on Dowex ion-exchange resin in order to obtain the peptide as the acetate salt.

Identification and purity determination of the target materials were carried out using an in-line analytical reverse phase HPLC with a cyano column (Daiso Fine Chem, SP-120-3-CN-P, 150×4.6 mm, 3 μm, 120 Å). The peptide was also analyzed by ESI LC-MS in Positive Ion Mode, using a Finnigan LCQ Deca XPMax.

Compounds B4, B6, B8, B12, B14 and B16 prepared and purified as described above, were found to be greater than 95% pure.

Experimental masses (m/z) accorded with calculated molecular weights for each compound.

### Peptide Quantitation

Quantitation of compounds was carried out by in vacuo hydrolysis at 110° C. of samples in sealed glass vials in the presence of 6N HCl containing 0.1% phenol. Derivatisation of amino acids was then carried out using Waters AccQTag reagents according to the manufacturer's instructions followed by analysis on a Waters Acquity UPLC System (Waters Millipore) using an AccQTag ultra column (2.1 mm×100 mm; Waters Millipore).

### 1.5—Synthesis of Sulfone and Sulfoxide Analogues of Compounds

Sulfone and sulfoxide derivatives of compounds of this invention may be accessed by a similar synthetic routes as described above, with the omission of ethylmethylsulfide scavenger, and optional omission of nitrogen sparging, from the carbamate formation step. This reaction may yield a mixture of thiol, sulfone and sulfoxide derivatives, which may be separated and purified by HPLC.

Alternatively, sulfone or sulfoxide derivatives may be prepared by oxidation of the corresponding sulfide with an oxidant such as meta-chloroperoxybenzoic acid (MCPBA) or tert-butyl hydroperoxide (t-BuOOH) under appropriate conditions.

### Example 2—Activation of Human TLR2

The potency of the compounds as activators of human and mouse TLR-2s is tested in an in vitro assay. The assay assesses NF-kB activation in the HEKBlue-mTLR-2 cell line. These cells have been stably transfected with mouse TLR-2 and express TLR-1 and TLR-6 endogenously at sufficient levels to allow for fully-functional TLR-1/2 and TLR-2/6 activation.

Toll-Like Receptor 2 (TLR2) stimulation is tested by assessing NF-kB activation in the HEKBlue-hTLR2 cell line. These cells have been stably transfected with human TLR2 and express TLR1 and TLR6 endogenously at a level sufficient to allow for fully-functional TLR1/2 and TLR2/6 activation. The activity of the test articles are tested on human TLR2 as potential agonists. The test articles are evaluated at seven concentrations and compared to control ligands. These steps are performed in triplicate.

NF-kB reporter gene assay protocol: This assay is carried out as described previously (Jackson et al. 2004; Lau et al. 2006; Sandor et al. 2003; Zeng et al 2010). HEK293T cells were cultured in 96-well plates at 4×10⁴ cells/well and transfected 24 h later with 100 ng of the NF-kB luciferase reporter gene [50 ng of TK-Renilla-luciferase expressing plasmid (Promega corporation, Madison, USA)] with or without 5 ng TLR2-expressing plasmid in the presence of 0.8 μl Fugene 6 (Roche Diagnostic). Compounds are added to the wells 24h later at the concentrations indicated in the histograms. Cell lysates are prepared 5 h after stimulation using reporter lysis buffer (Promega Corporation, Madison, USA). Luciferase activities in the cell lysates were determined using a reagent kit (Promega Corporation, Madison, USA) and using a FLUOstar microplate reader (BMG Labtech, Ortenberg, Germany). The NF-kB-dependent firefly luciferase activity is normalised with NF-kB-independent renilla luciferase activity. The relative stimulation is calculated as the ratio of the stimulated to non-stimulated samples.

### Example 3—URT Virus Challenge

In these Examples, an upper respiratory tract (URT) influenza virus challenge model is utilised in mice, using a dose of infectious virus which replicates in the URT and then progress to the lungs. The URT model is used to determine which compounds can prevent replication and dissemination of influenza virus from the URT to the lungs.

Cytokine and chemokine profiles in the nasal turbinates, trachea, lungs and sera of animals following URT treatment with three doses or a single dose of the compounds are also measured.

The cytokine profiles of mice which were pre-treated with three doses of compounds of the invention followed by challenge with Udorn virus are also measured.

### Experimental Animals

Groups of male or female C57BL/6 mice of similar age (e.g. about 6-8 week old) are used for all studies. After administration of saline, the compound or viral challenge, mice are monitored daily for weight changes, and behavioural or physical changes.

### URT Administration of Compounds

Mice are anaesthetized by isoflurane inhalation and saline or various doses of the compounds, diluted in saline, are administered intranasally using a pipettor. For the multi-treatment experiments, mice receive 3 doses of the compounds of the invention every second day over a 5 day period.

### Preparation of Influenza Virus

A/Udorn/307/72 (H3N2) influenza virus (ie. Udorn virus) is propagated in the allantoic cavity of 10 day-old embryonated hens' eggs. Eggs are inoculated with approximately 10³ pfu of virus in 0.1 ml of saline. After 2 days incubation at 35° C. the eggs are chilled at 4° C. and allantoic fluid harvested and clarified by centrifugation. Viral infectivity titre (pfu/mL) is determined by plaque assay as described below and aliquots of the allantoic fluid were stored at −80° C. until used.

### URT Virus Challenge

Mice are anaesthetised with isofluorane and inoculated intranasally with 500 pfu of Udorn virus in 10 μl of saline, using a pipettor. On day 5 post-challenge, the nasal turbinates, trachea and lung are harvested to assess viral loads.

### Extraction and Preparation of Nasal Turbinates, Trachea and Lung Homogenates

Mice are killed by CO2 asphyxiation 24 hours post-treatment or 5 days post-influenza challenge. Nasal turbinates, trachea and lungs from each mouse are collected in 1.5 mL of RPMI-1640 medium with antibiotics (100 ug/mL penicillin, 180 ug/mL streptomycin and 24 ug/mL gentamicin) and kept on ice until processed. Tissues were homogenised using a tissue homogeniser and the resulting organ homogenates then centrifuged at 2,000 rpm for 5 min to remove cell debris. Supernatants are collected and stored at −80° C. for subsequent measurements.

### Assessment of Viral Titres

Titres of infectious Udom virus are determined by plaque assay on confluent monolayers of Madin Darby canine kidney (MDCK) cells. Six-well tissue culture plates were seeded with 1.2×10⁶ MDCK cells per well in 3 ml of RP10 (RPMI-1640 medium supplemented with 10% (v/v) heat inactivated FCS, 260 ug/mL glutamine, 200 ug/mL sodium pyruvate and antibiotics). After overnight incubation at 37° C. in 5% CO₂ confluent monolayers were washed with RPMI. Test supernatants serially diluted in RPMI with antibiotics, are added to duplicate wells of monolayers. After incubation at 37° C. in 5% CO₂ for 45 min, monolayers are overlaid with 3 mL of agarose overlay medium containing 0.9% agarose and 2 ug/mL trypsin-TPCK treated in Leibovitz L15 medium pH6.8 with glutamine and antibiotics. Plates are incubated for 3 days at 37° C. in 5% CO₂ and virus-mediated cell lysis then counted as plaques on the cell layer. The total organ viral titres (plaque forming units, PFU) for individual animals are calculated.

### Determination of Cytokine Levels in Nasal Turbinates, Trachea, Lungs and Sera

IFN-γ, IL-2, IL-4, TNF, IL-10, IL-6, KC, MCP-1, RANTES, IL-12/IL-23p40 and IL-17A present in nasal turbinates, trachea, lung homogenates and serum samples were measured using a BD Cytometric Bead Array (CBA) Flex Kit according to the manufacturer's instructions with the exception that a total of 0.15 μl of each capture bead suspension and 0.15 μl of each PE-detection reagent is used in each 50 μl sample. Samples were analysed using a Bection Dickinson FACSCanto II flow cytometer and the data analysed using FCAP Array multiplex software.

### Statistical Analyses

A one-way analysis of variance (ANOVA) with Tukey comparison of all column tests may be used. A two-way ANOVA with Bonferroni's test may be used to compare the same treatment groups in the single and 3 repeat dose regimes. A β-value≤0.0322 was considered statistically significant. Statistical analyses are performed using suitable software, such as GraphPad Prism, version 7.0.

### Example 4—Assessing the Effect of Pre-Treatment with Different Doses of Compounds of the Invention on the Outcome of URT Challenge with Udorn Virus

This experiment is performed to determine the anti-viral effect of URT pre-treatment with various doses of the compounds of the invention.

On day 0 mice (5 animals/group) receive either saline, 5 nmoles, 0.1 nmoles or 0.005 nmoles of compound of the invention, administered intranasally in 10 μl after being anaesthetized with isoflurane. On day 1 following administration with compound of the invention, mice are challenged intranasally with 500 pfu of Udorn virus in a volume of 10 μl after being anaesthetized with isoflurane. Mice are killed on day 5 and nasal turbinates trachea and lungs were removed, homogenised and frozen for subsequent analyses.

The experimental design is summarised in the schematic below

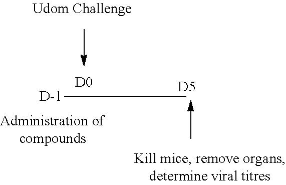

### Example 5—TLR2 Activation by Various Compounds

Comparison of the abilities of various compounds to stimulate luciferase activity in an NF-κB cell-based reporter system is determined. HEK293T cells, transiently co-transfected with a human TLR2 plasmid and a luciferase-NF-κB plasmid reporter system, are exposed to various dilutions of each compound. Successful receptor binding and subsequent signal transduction events are determined by measuring the luminescence due to luciferase activity

### Example 6—TLR Binding and Specificity

The compound of the invention is assessed for its ability to activate a range of other TLR pattern recognition receptors. These assessments are conducted using both human and mouse TLR panels. These assays detect a secreted embryonic alkaline phosphatase (SEAP) reporter under the control of a promoter which is inducible by NF-κB activation in HEK293 cells.

The secreted embryonic alkaline phosphatase (SEAP) reporter is under the control of a promoter inducible by the transcription factor NF-κB. This reporter gene allows the monitoring of signaling through the TLR, based on the activation of NF-κB. In a 96-well plate (200 μL total volume) containing the appropriate cells (50,000-75,000 cells/well), 20 μL of the test article or the positive control ligand is added to the wells. The media added to the wells is designed for the detection of NF-κB induced SEAP expression. After a 16-24 hr incubation the optical density (OD) is read at 650 nm on a Molecular Devices SpectraMax 340PC absorbance detector.

### Control Ligands

hTLR2: HKLM (heat-killed Listeria monocytogenes) at 1×108 cells/mL
hTLR3: Poly(I:C) HMW at 1 μg/mL
hTLR4: E. coli K12 LPS at 100 ng/mL
hTLR5: S. typhimurium flagellin at 100 ng/mL
hTLR7: CL307 at 1 μg/mL
hTLR8: CL075 at 1 μg/mL
hTLR9: CpG ODN2006 at 1 μg/mL.

### Example 7—Stability I

Stability is assessed by tracking changes in the absolute peak area and % peak area of the compound subjected to the following conditions to the peak area and % peak area obtained from freshly prepared solutions of the relevant compound. The compound is formulated in each of the following formulations:
2. Phosphate buffered saline (PBS), pH 7.4. For example, the PBS buffer may comprise 8 g NaCl, 0.2 g KCl, 1.15 g disodium hydrogen phosphate and 0.2 g potassium dihydrogen phosphate in 1 litre of MilliQ water.
3. 0.9% w/w saline (pH 5.8). For example, saline solution may be prepared by dissolving sodium chloride (1.855 g) in 200 mL of Milli-Q water.

Stability for each formulation is assessed under the following conditions:
1. 25° C./60% relative humidity (ICH ambient)
2. 40° C./75% relative humidity (ICH accelerated)

### Sample Preparation

Solutions of approximately 1 mg/mL of each compound (2 mL) are accurately prepared in the PBS and saline diluent systems.

All compounds are heated to approximately 60° C. under hot running tap water for approximately 30 seconds, followed by vortex mixing for a further 30 seconds, and then are further sub-aliquoted into 3 separate HPLC vials which are then placed into storage at 4-8° C. (fridge), 25° C./65% RH and 40° C./75% relative humidity (RH) for 2 weeks. The vials are wrapped in aluminium foil to exclude light for the storage duration.

### Equipment and Operational Parameters

A Shimadzu Nexera UHPLC with diode array detector is used to monitor peak area changes at t=0 and t=2 weeks.

A Shimadzu LCMS-8030 system is used to identify impurity and degradant peaks, and to verify the selectivity of the HPLC methods by checking across the main HPLC peak for possible co-eluting components. Exemplary ultra high-performance liquid chromatography (UHPLC) parameters are outlined below.

### UHPLC Parameters

Column—Phenomenex Kinetex Biphenyl, 50×2.1 mm, 2.6 μm, part no. 00B-4622-AN
Vials—Agilent clear glass, 2 mL with multi-injection septa, part no. 226-50512-00
Mobile Phase A—5 mM ammonium formate in Milli-Q water
Mobile Phase B—acetonitrile, Merck LC-MS grade
Needle Rinse Solution—1:1 water:methanol
Injection Volume: 1 μL
Column Temperature: 40° C.
Autosampler Temp: 20° C.
Total Flow Rate: 0.5 mL/min
Total Run Time: 10 min
UV-vis wavelength: 205 nm

TABLE 1
Gradient 1

| Time (min) | % A | % B |
|---|---|---|
| Init | 55 | 45 |
| 0.1 | 55 | 45 |
| 8.0 | 25 | 75 |
| 8.5 | 25 | 75 |
| 8.6 | 55 | 45 |

TABLE 2
Gradient 2

| Time (min) | % A | % B |
|---|---|---|
| Init | 55 | 45 |
| 0.1 | 55 | 45 |
| 8.0 | 25 | 65 |
| 8.5 | 25 | 65 |
| 8.6 | 55 | 45 |

### LCMS Parameters

LC injection volume: 0.1 μL
Interface: ESI
Interface Temperature: 350° C.
Desolvation Temperature: 250° C.
Nebuliser Flow: 3 L/min
Heat Block: 400° C.
Drying Gas Flow: 15 L/min
Q3 scan mode: Positive
Start Time: 1 min
End Time: 8 min
Start m/z: 400
End m/z 2000
Scan Speed: 15000 μ/sec

### Example 8—Stability II

The relative stabilities of compound B12 and that of compound (8) of WO2019/119067 (compound A107) were evaluated under accelerated conditions (40° C./75% RH) for 9 days. Each compound was prepared at 1 mg/mL concentration in an aqueous formulation of 0.1% w/v ethylenediaminetetraacetic acid (EDTA)/0.9% saline w/v buffered to pH 5.

Stability was measured using reversed phase HPLC with a UV detector analytical wavelength of 205 nm. Peak areas of each compound at the 9 day time point were compared with areas at time zero. Compound stability at day 9 was calculated as a percentage of the time zero peak area data.

Compound stability was further assessed by comparison with a reference sample of the same compound. Reference samples were prepared at 1 mg/mL concentration in an aqueous formulation of 0.1% w/v EDTA/0.9% saline w/v buffered to pH 5 and frozen during the period of testing. Thawed samples were sonicated and measured by HPLC.

Results are summarised in Table 3.

TABLE 3

| | | % RSD (relative | | |
|---|---|---|---|---|
| | area % recovery | standard deviation) | % RSD in | Estimated total |
| Compound | (Day 9) | in reference | samples | % RSD (RMS) |
| Compound (8) of | 94.4 | 0.01 | 0.02 | 0.02 |
| WO2019/119067 | | | | |
| B12 | 98.4 | 0.00 | 0.23 | 0.23 |

Compound B12 is shown to possess substantial stability over the 9 day test period. Compound B12 also possesses superior stability under the accelerated conditions than the comparator compound.

### Example 9—Activation of Human TLR2 II

The potency of the compounds as activators of human TLR-2s is tested in an in vitro assay in HEK-BLUE-hTLR2 cells.

### Culturing of HEK-BLUE-hTLR2 Cells

HEK-BLUE-hTLR2 cells are designed for studying the stimulation of human TLR2 (hTLR2) by monitoring the activation of NF-kB. HEK-BLUE-hTLR2 cells are obtained by co-transfection of the hTLR2 and SEAP (secreted embryonic alkaline phosphatase) reporter genes into HEK293 cells. Stimulation with a TLR2 ligand activates NF-kB which induces the production of SEAP.

HEK-BLUE-hTLR2 cells were purchased from InvivoGen (San Diego, CA, USA). Cells were grown in DMEM supplemented with 10% FCS, 100U/ml penicillin, 100 ug/ml streptomycin and 2 mM L-glutamine, 100 μg/mL Normocin in the presence of selection antibiotic purchased from InvivoGen and passaged when 70% confluence was reached per manufacturer's recommendation. Cells were dislodged and resuspended in test media as suggested by manufacturer for testing.

### Testing of Compounds

i) A serial dilution of respective compounds were prepared in saline and added in 20 ml of each dilution in triplicates per well in a flat bottom 96-well plate and placed in the incubator while waiting for the cells.
ii) Remove HEK-BLUE-hTLR2 cells in a T-75 flask from incubator and discard the growth media.
iii) Gently rinse the cells with prewarmed 10 ml of PBS
iv) Add 5 ml of prewarmed PBS and place the cells in 37° C. for 2 mins and then detach the cells by gently pipetting up and down the PBS on the surface where the cells adhere.
v) Cells suspension at the density of 280,000 cells/ml is prepared in HEK-Blue™ Detection medium which is purchased from InvivoGen and prepared according to the manufacturer's instruction,
vi) Add immediately 180 ml of the cell suspension per well of the plate which contains the solution of the compounds. The plate is then returned to the incubator at 37° C. for 16 hr and was read at 620 nm by using an ELISA reader.

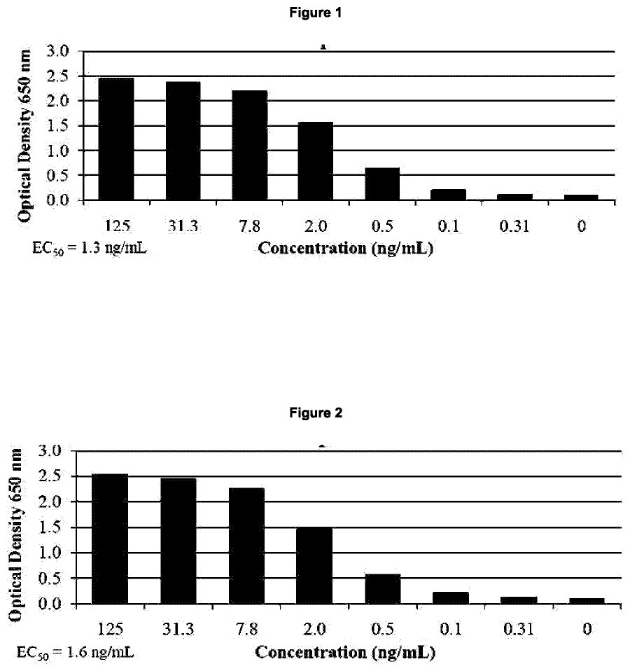

The results of this assay for compounds B4 and B12 are outlined in Tables 4 (B4) and 5 (B12) and shown in FIG. 1 (B4) and FIG. 2 (B12). These data show that the compounds B4 and B12 exhibit significant activity at TLR2, where the EC₅₀ of compound B4 is 1.3 ng/mL and the EC₅₀ of compound B12 is 1.6 ng/mL.

TABLE 4
Human TLR2 dose response for compound B4.

| | Compound B4 (ng/mL) | | | | | | | |
|---|---|---|---|---|---|---|---|---|
| Screening | 125 | 31.3 | 7.8 | 2.0 | 0.5 | 0.1 | 0.31 | 0 |
| 1 | 2.451 | 2.438 | 2.214 | 1.660 | 0.719 | 0.182 | 0.126 | 0.093 |
| 2 | 2.411 | 2.327 | 2.163 | 1.529 | 0.594 | 0.206 | 0.115 | 0.115 |
| 3 | 2.467 | 2.350 | 2.214 | 1.498 | 0.617 | 0.197 | 0.115 | 0.101 |

Fold induction*

| | | | | | | | |
|---|---|---|---|---|---|---|---|
| 23.7 | 23.0 | 21.3 | 15.2 | 6.2 | 1.9 | 1.2 | 1.0 |

Notes:
Results are provided as optical density values (650 nm)
*Ratio of average induced value to average non-induced value

TABLE 5
Human TLR2 dose response for compound B12.

| | Compound B12 (ng/mL) | | | | | | | |
|---|---|---|---|---|---|---|---|---|
| Screening | 125 | 31.3 | 7.8 | 2.0 | 0.5 | 0.1 | 0.31 | 0 |
| 1 | 2.409 | 2.422 | 2.187 | 1.429 | 0.585 | 0.239 | 0.116 | 0.104 |
| 2 | 2.564 | 2.423 | 2.257 | 1.308 | 0.558 | 0.202 | 0.132 | 0.093 |
| 3 | 2.615 | 2.449 | 2.302 | 1.638 | 0.559 | 0.208 | 0.137 | 0.094 |
| | Fold induction* | | | | | | | |
| | 26.1 | 25.1 | 23.2 | 15.0 | 5.8 | 2.2 | 1.3 | 1.0 |

Notes:
Results are provided as optical density values (650 nm)
*Ratio of average induced value to average non-induced value

### Example 9—Activation of Human TLR2 III

Toll-Like Receptor (TLR) stimulation is tested by assessing NF-κB activation in the TLR expressing cell lines. HEK-Blue h/mTLR2 cells have been stably transfected with human or mouse TLR2 and CD14. The activity of the test articles are tested on human and mouse TLR2, as potential agonists. The test articles are evaluated at seven concentrations and compared to control ligands (see list below). These steps are performed in triplicate.

### Control Ligands

HEK-Blue hTLR2 Dose Response:
HKLM (heat-killed Listeria monocytogenes) at 1.0×10⁸, 2.5×10⁷, 6.25×10⁶, 1.56×10⁶, 3.91×10⁵, 9.76×10⁴ and 2.44×10⁴ cells/mL

HEK-Blue mTLR2 Dose Response:
HKLM (heat-killed Listeria monocytogenes) at 1.0×10⁸, 2.5×10⁷, 6.25×10⁶, 1.56×10⁶, 3.91×10⁵, 9.76×10⁴ and 2.44×10⁴ cells/mL

### TLR—Control Cell Lines

HEK-Blue Null1 Dose Response:
TNFα at 100, 25, 6.25, 1.56, 0.39, 0.098 and 0.024 ng/mL
Control for human TLR2

HEK-Blue Null2 Dose Response:
TNFα at 100, 25, 6.25, 1.56, 0.39, 0.098 and 0.024 ng/mL
Control for mouse TLR2

### Test Articles and Materials

| Article 1: | B4 | |
|---|---|---|
| Weight: | 2.2 | mg |
| Resuspension: | 1.1 | mL PBS |
| Stock Concentration: | 2 | mg/mL |
| Final Concentrations: | 500, 125, 31.25, 7.81, 1.95, 0.49 and 0.12 ng/ml | |
| Storage Condition: | −20° | C. |

| Article 2: | B6 | |
|---|---|---|
| Weight: | 2.4 | mg |
| Resuspension: | 1.2 | mL PBS |
| Stock Concentration: | 2 | mg/mL |
| Final Concentrations: | 500, 125, 31.25, 7.81, 1.95, 0.49 and 0.12 ng/ml | |
| Storage Condition: | −20° | C. |

| Article 3: | B8 | |
|---|---|---|
| Weight: | 2.4 | mg |
| Resuspension: | 1.2 | mL PBS |
| Stock Concentration: | 2 | mg/mL |
| Final Concentrations: | 500, 125, 31.25, 7.81, 1.95, 0.49 and 0.12 ng/ml | |
| Storage Condition: | −20° | C. |

| Article 4: | B14 | |
|---|---|---|
| Weight: | 2.6 | mg |
| Resuspension: | 1.3 mL PBS | |
| Stock Concentration: | 2 | mg/mL |
| Final Concentrations: | 500, 125, 31.25, 7.81, 1.95, 0.49 and 0.12 ng/mL | |
| Storage Condition: | −20° | C. |

| Article 5: | B12 | |
|---|---|---|
| Weight: | 2.1 | mg |
| Resuspension: | 1.05 mL PBS | |
| Stock Concentration: | 2 | mg/mL |
| Final Concentrations: | 500, 125, 31.25, 7.81, 1.95, 0.49 and 0.12 ng/mL | |
| Storage Condition: | −20° | C. |

| Article 6: | B16 | |
|---|---|---|
| Weight: | 2.2 | mg |
| Resuspension: | 1.1 mL PBS | |
| Stock Concentration: | 2 | mg/mL |
| Final Concentrations: | 500, 125, 31.25, 7.81, 1.95, 0.49 and 0.12 ng/mL | |
| Storage Condition: | −20° | C. |

### Preparation of Test Articles

A series of two 1:10 serial dilutions are prepared in sterile PBS starting from the 2 mg/mL stock solution of each of compounds B4, B6, B8, B12, B14 and B16 and ending with 20 μg/mL. A 5 μg/mL working stock was then prepared for each compound from the 20 μg/mL dilution in sterile PBS. Starting from the 5 μg/mL working stock, a series of six 1:4 serial dilutions were made by mixing 100 μL of the previous highest dilution with 300 μL sterile PBS.

### General Procedure

The secreted embryonic alkaline phosphatase (SEAP) reporter is under the control of a promoter inducible by the transcription factor NF-κB. This reporter gene allows the monitoring of signaling through the TLR, based on the activation of NF-κB. In a 96-well plate (200 μL total volume) containing the appropriate cells (50,000-75,000 cells/well), 20 μL of the test article or the positive control ligand (HKLC) is added to the wells. The media added to the wells is designed for the detection of NF-κB induced SEAP expression. After a 16-24-hour incubation the optical density (OD) is read at 650 nm on a Molecular Devices SpectraMax 340PC absorbance detector.

### Results

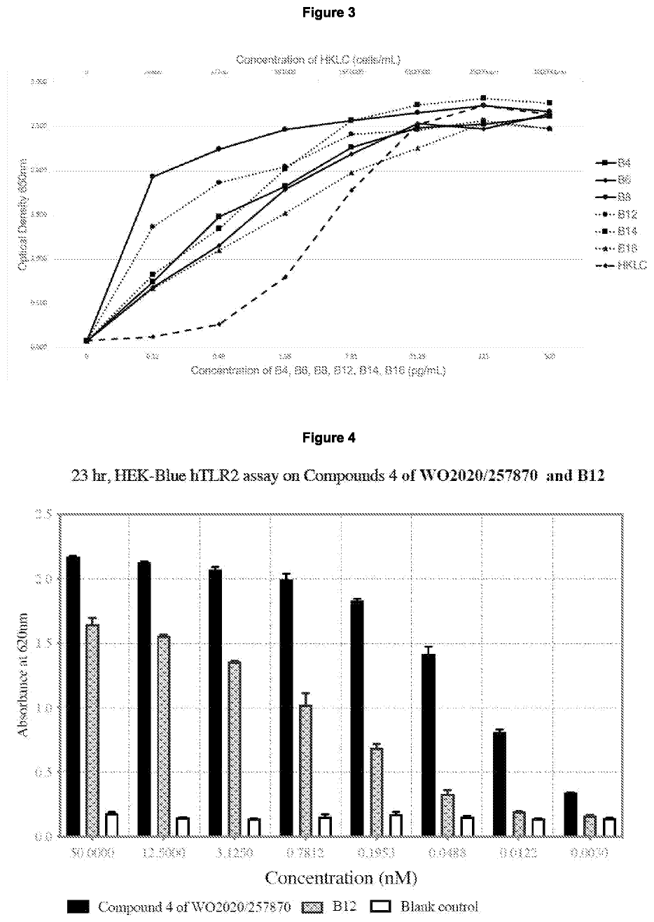

All test articles showed TLR2 agonist activity for both human TLR2 (hTLR2) and mouse TLR2 (mTLR2). Results for hTLR agonist are shown in Tables 6 to 12 and FIG. 3.

TABLE 6
HEK-Blue hTLR2 Dose Response for compound B4. Results
are provided as optical density values (650 nm)

| | Concentration (pg/mL) | | | | | | | |
|---|---|---|---|---|---|---|---|---|
| Screening | 0 | 0.12 | 0.49 | 1.95 | 7.81 | 31.25 | 125 | 500 |
| 1 | 0.071 | 0.653 | 1.409 | 1.795 | 2.288 | 2.440 | 2.569 | 2.717 |
| 2 | 0.072 | 0.759 | 1.507 | 1.757 | 2.168 | 2.485 | 2.508 | 2.590 |
| 3 | 0.074 | 0.809 | 1.521 | 1.916 | 2.323 | 2.518 | 2.487 | 2.526 |
| Average | 0.072 | 0.740 | 1.479 | 1.823 | 2.260 | 2.481 | 2.521 | 2.611 |
| | Fold Induction* | | | | | | | |
| | 1.0 | 10.3 | 20.5 | 25.3 | 31.4 | 34.5 | 35.0 | 36.3 |

*Ratio of average induced value to average non-induced value.

TABLE 7
HEK-Blue hTLR2 Dose Response for compound B6. Results
are provided as optical density values (650 nm)

| | Concentration (pg/mL) | | | | | | | |
|---|---|---|---|---|---|---|---|---|
| Screening | 0 | 0.12 | 0.49 | 1.95 | 7.81 | 31.25 | 125 | 500 |
| 1 | 0.069 | 0.591 | 1.230 | 1.755 | 2.154 | 2.512 | 2.522 | 2.649 |
| 2 | 0.072 | 0.742 | 1.151 | 1.776 | 2.186 | 2.527 | 2.466 | 2.606 |
| 3 | 0.074 | 0.704 | 1.088 | 1.824 | 2.219 | 2.547 | 2.422 | 2.656 |
| Average | 0.072 | 0.679 | 1.156 | 1.785 | 2.186 | 2.529 | 2.470 | 2.637 |
| | Fold Induction* | | | | | | | |
| | 1 | 9.4 | 16.1 | 24.8 | 30.4 | 35.1 | 34.3 | 36.6 |

*Ratio of average induced value to average non-induced value.

TABLE 8
HEK-Blue hTLR2 Dose Response for compound B8. Results
are provided as optical density values (650 nm)

| | Concentration (pg/mL) | | | | | | | |
|---|---|---|---|---|---|---|---|---|
| Screening | 0 | 0.12 | 0.49 | 1.95 | 7.81 | 31.25 | 125 | 500 |
| 1 | 0.072 | 1.936 | 2.345 | 2.469 | 2.326 | 2.563 | 2.746 | 2.648 |
| 2 | 0.072 | 1.856 | 2.229 | 2.420 | 2.636 | 2.643 | 2.716 | 2.664 |
| 3 | 0.083 | 1.992 | 2.154 | 2.507 | 2.734 | 2.751 | 2.743 | 2.693 |
| Average | 0.076 | 1.928 | 2.243 | 2.465 | 2.565 | 2.652 | 2.735 | 2.668 |
| | Fold Induction* | | | | | | | |
| | 1 | 25.4 | 29.5 | 32.4 | 33.8 | 34.9 | 36 | 35.1 |

*Ratio of average induced value to average non-induced value.

TABLE 9
HEK-Blue hTLR2 Dose Response for compound B12. Results
are provided as optical density values (650 nm)

| | Concentration (pg/mL) | | | | | | | |
|---|---|---|---|---|---|---|---|---|
| Screening | 0 | 0.12 | 0.49 | 1.95 | 7.81 | 31.25 | 125 | 500 |
| 1 | 0.071 | 1.443 | 1.892 | 2.149 | 2.420 | 2.492 | 2.608 | 2.584 |
| 2 | 0.072 | 1.313 | 1.785 | 2.008 | 2.446 | 2.500 | 2.541 | 2.404 |
| 3 | 0.077 | 1.329 | 1.902 | 1.979 | 2.366 | 2.380 | 2.555 | 2.426 |
| Average | 0.073 | 1.362 | 1.860 | 2.045 | 2.411 | 2.457 | 2.568 | 2.471 |
| | Fold Induction* | | | | | | | |
| | 1 | 18.7 | 25.5 | 28 | 33 | 33.7 | 35.2 | 33.9 |

*Ratio of average induced value to average non-induced value.

TABLE 10
HEK-Blue hTLR2 Dose Response for compound B14. Results
are provided as optical density values (650 nm)

| | Concentration (pg/mL) | | | | | | | |
|---|---|---|---|---|---|---|---|---|
| Screening | 0 | 0.12 | 0.49 | 1.95 | 7.81 | 31.25 | 125 | 500 |
| 1 | 0.073 | 0.826 | 1.256 | 1.994 | 2.569 | 2.820 | 2.928 | 2.722 |
| 2 | 0.073 | 0.869 | 1.364 | 2.061 | 2.545 | 2.720 | 2.800 | 2.765 |
| 3 | 0.072 | 0.774 | 1.406 | 2.000 | 2.571 | 2.691 | 2.713 | 2.791 |
| Average | 0.073 | 0.823 | 1.342 | 2.018 | 2.562 | 2.744 | 2.814 | 2.759 |
| | Fold Induction* | | | | | | | |
| | 1 | 18.7 | 25.5 | 28 | 33 | 33.7 | 35.2 | 33.9 |

*Ratio of average induced value to average non-induced value.

TABLE 11
HEK-Blue hTLR2 Dose Response for compound B16. Results
are provided as optical density values (650 nm)

| | Concentration (pg/mL) | | | | | | | |
|---|---|---|---|---|---|---|---|---|
| Screening | 0 | 0.12 | 0.49 | 1.95 | 7.81 | 31.25 | 125 | 500 |
| 1 | 0.071 | 0.688 | 1.080 | 1.478 | 1.903 | 2.093 | 2.531 | 2.466 |
| 2 | 0.074 | 0.591 | 1.097 | 1.513 | 1.897 | 2.297 | 2.544 | 2.488 |
| 3 | 0.072 | 0.718 | 1.121 | 1.573 | 2.133 | 2.367 | 2.545 | 2.472 |
| Average | 0.072 | 0.666 | 1.099 | 1.521 | 1.978 | 2.252 | 2.540 | 2.475 |
| | Fold Induction* | | | | | | | |
| | 1 | 9.2 | 15.3 | 21.1 | 27.5 | 31.3 | 35.3 | 34.4 |

*Ratio of average induced value to average non-induced value.

TABLE 12
HEK-Blue hTLR2 Dose Response for HKLM. Results
are provided as optical density values (650 nm)

| | Concentration (pg/mL) | | | | | | | |
|---|---|---|---|---|---|---|---|---|
| Screening | 0 | 24400 | 97700 | 391000 | 1560000 | 6200000 | 25000000 | 100000000 |
| 1 | 0.079 | 0.124 | 0.260 | 0.826 | 1.809 | 2.574 | 2.777 | 2.607 |
| 2 | 0.081 | 0.124 | 0.253 | 0.769 | 1.866 | 2.605 | 2.747 | 2.574 |
| 3 | 0.079 | 0.118 | 0.271 | 0.783 | 1.653 | 2.382 | 2.681 | 2.715 |
| Average | 0.080 | 0.122 | 0.261 | 0.793 | 1.776 | 2.520 | 2.735 | 2.632 |
| | Fold Induction* | | | | | | | |
| | 1 | 1.5 | 3.3 | 10 | 22.5 | 31.9 | 34.6 | 33.3 |

*Ratio of average induced value to average non-induced value.

TABLE 13
EC50 values for agonism of hTLR2

| Compound | EC₅₀ (hTLR2) |
|---|---|
| B4 | 0.46 ng/mL |
| B6 | 0.73 ng/mL |
| B8 | n.d. |
| B12 | 0.11 ng/mL |
| B14 | 0.57 ng/mL |
| B16 | 1.09 ng/mL |
| HKLM | 9.24 × 10⁵ cells/mL |

n.d. = EC₅₀ value could not be calculated due to lack of confidence in extrapolation of sigmoidal response curve due to high activity at lowest concentration tested.

### Conclusions

Each of compounds B4, B6, B8, B12, B14 and B16 possesses stimulatory activity for hTLR2 and mTLR2 in the described HEK-Blue assay. The stimulatory response was not observed in HEK-Blue Null1 (human) or HEK-Blue Null2 (mouse) cells, confirming that the compounds' efficacy is mediated by hTLR2 or mTLR2.

### Example 10—Activation of Human TLR2 IV

The potency of compound 4 of WO2020/257870 (Compound A204) and compound B12 as activators of human TLR-2s is tested in an in vitro assay in HEK-BLUE-hTLR2 cells.

### Culturing of HEK-BLUE-hTLR2 Cells

HEK-BLUE-hTLR2 cells are designed for studying the stimulation of human TLR2 (hTLR2) by monitoring the activation of NF-kB. HEK-BLUE-hTLR2 cells are obtained by co-transfection of the hTLR2 and SEAP (secreted embryonic alkaline phosphatase) reporter genes into HEK293 cells. Stimulation with a TLR2 ligand activates NF-kB which induces the production of SEAP.

HEK-BLUE-hTLR2 cells were purchased from InvivoGen (San Diego, CA, USA). Cells were grown in DMEM supplemented with 10% FCS, 100 U/ml penicillin, 100 ug/ml streptomycin and 2 mM L-glutamine, 100 μg/mL Normocin in the presence of selection antibiotic purchased from InvivoGen and passaged when 70% confluence was reached per manufacturer's recommendation. Cells were dislodged and resuspended in test media as suggested by manufacturer for testing.

### Testing of Compounds

vii) A serial dilution of respective compounds were prepared in saline and added in 20 ml of each dilution in triplicates per well in a flat bottom 96-well plate and placed in the incubator while waiting for the cells.
viii) Remove HEK-BLUE-hTLR2 cells in a T-75 flask from incubator and discard the growth media.
ix) Gently rinse the cells with prewarmed 10 ml of PBS
x) Add 5 ml of prewarmed PBS and place the cells in 37° C. for 2 mins and then detach the cells by gently pipetting up and down the PBS on the surface where the cells adhere.
xi) Cells suspension at the density of 280,000 cells/ml is prepared in HEK-Blue™ Detection medium which is purchased from InvivoGen and prepared according to the manufacturer's instruction,
xii) Add immediately 180 ml of the cell suspension per well of the plate which contains the solution of the compounds. The plate is then returned to the incubator at 37° C. for 16 hr and was read at 620 nm by using an ELISA reader.

The results of this assay for compound 4 of WO2020/257870 and compound B12 are shown in FIG. 4. These data show that these compounds exhibit significant activity at TLR2.

### Example 11—Stability III

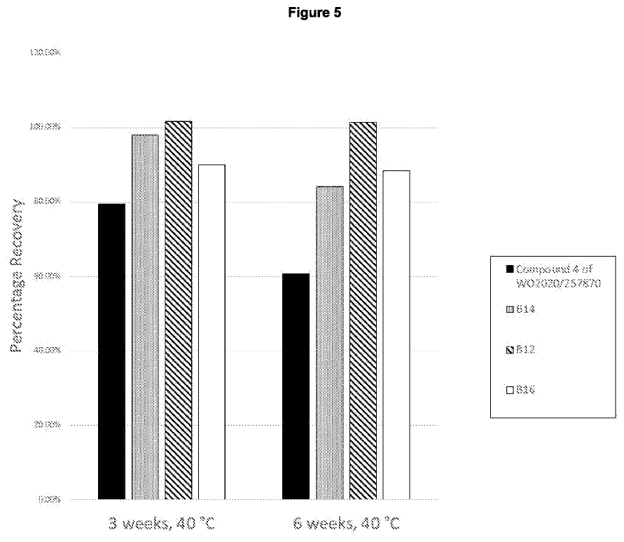

Following a similar procedure to that described in Example 8, compounds B12, B14, B16 and compound 4 of WO2020/257870 (Compound A204) were subjected to accelerated aging conditions at 40° C. for either 3 weeks or 6 weeks. Recovery of the target compound was assessed by HPLC, and the values as a percentage of the reference sample are reported in Table 14 and in FIG. 5.

TABLE 14
Percentage recovery results at 40° C.

| | Recovery compared to reference sample (%) | |
|---|---|---|
| Compound ID | 3 weeks | 6 weeks |
| Compound 4 of | 79.40% | 60.70% |
| WO2020/257870 (A204) | | |
| B14 | 97.91% | 83.99% |
| B12 | 101.60% | 101.33% |
| B16 | 89.97% | 88.35% |

These data show that each of compounds B12, B14 and B16 possess excellent stabilities under the accelerated storage conditions. Further, each of compounds B12, B14 and B16 demonstrated improved storage stability compared with compound A204.

### Example 12—Stability IV

Following a similar procedure to that described in Example 8, compound 8 of WO2019/119067 (Compound A107), compound 4 of WO2020/257870 (Compound A204), and compounds B4 and B12 were assessed for storage stability under accelerated conditions over a period of 28 days.

Each compound was formulated in 0.9% w/w saline with addition of 0.1% w/w EDTA at about pH 5. The formulations were stored in the dark under accelerated conditions (40° C./75% RH) and reference formulations were stored at −80° C. At days 0, 9 and 28, samples were analysed by UHPLC and pH was measured.

This experiment shows that following storage under the accelerated conditions after 28 days, B4 and B12 are the most stable compounds.

### Sample Preparation

Approximately 1.5 mg of each compound was weighed into glass HPLC vials and 1.5 mL of saline/EDTA formulation adjusted to pH 5.0 was added. The vials were vortex mixed then warmed under running hot water until full dissolution was observed. Actual weights and concentrations of compounds are presented in Table 15.

TABLE 15
Weights and concentrations of formulated compound

| | | | Concentration |
|---|---|---|---|
| Compound | Mass (mg) | Final Volume (mL) | (mg/mL) |
| A107 | 1.658 | 1.5 | 1.1 |
| A204 | 1.566 | 1.5 | 1.0 |
| B12 | 1.758 | 1.5 | 1.2 |
| B4 | 1.627 | 1.5 | 1.1 |

4×100 μL aliquots of each solution were transferred to Agilent polypropylene 200 μL HPLC vials for storage under accelerated and reference conditions (two aliquots at each condition). These vials were removed at each time point for HPLC analysis. The frozen reference solutions at Day 9 and Day 28 were gently warmed under running hot water followed by vortex mixing for ˜10 sec and sonication for 10 min to encourage complete dissolution.

The remaining ˜1 mL of solution in the clear glass HPLC vials was stored at 40° C./75% RH and was used to measure pH at each time point.

### UHPLC Analysis

UHPLC analysis of compounds A107, A204, B4 and B12, was performed using the following conditions:
Column: Phenomenex Kinetex Biphenyl, 50 mm×2.1 mm, 2.6 μm, part no. 00B-4622-AN
Mobile Phase A: 5 mM ammonium formate in Milli-Q water, pH unadjusted
Mobile Phase B: acetonitrile, Merck LC-MS grade
Needle Rinse Solution: 1:1 water:methanol
Injection Volume: 5 μL
Column Temperature: 40° C.
Autosampler Temp: 20° C.
Total Flow Rate: 0.5 mL/min
Total Run Time: 10 min
UV-vis wavelength: 205 nm

Compounds A107 and A204 were analysed according to Gradient 1:

TABLE 16
Gradient 1

| Time (min) | % A | % B |
|---|---|---|
| Init | 55 | 45 |
| 0.1 | 55 | 45 |
| 8.0 | 25 | 75 |
| 8.5 | 25 | 75 |
| 8.6 | 55 | 45 |
| 10.0 | 55 | 45 |

Compounds B4 and B12 were analysed according to Gradient 2:

TABLE 17
Gradient 2

| Time (min) | % A | % B |
|---|---|---|
| Init | 55 | 45 |
| 0.1 | 55 | 45 |
| 7.0 | 25 | 80 |
| 8.5 | 25 | 80 |
| 8.6 | 55 | 45 |
| 10.0 | 55 | 45 |

### UHPLC Results

UHPLC results were analysed in a similarly to that described in Example 8. Results are provided as percentage recovery of main peak area (Table 18).

Percentage recovery of main peak area is calculated according to the following equation (1):

$$Percent Recovery = \frac{Area of Main Peak ( accelerated )}{Area of Main Peak ( frozen )} \times 100 \qquad ( 1 )$$

TABLE 18
Stability results calculated as percentage recovery of main peak area

| | Day 0 | | Day 9 | | Day 28 | |
|---|---|---|---|---|---|---|
| | | Standard | | Standard | | Standard |
| | % | Error | % | Error | % | Error |
| | Recovery | (% RSD) | Recovery | (% RSD) | Recovery | (% RSD) |
| Compound 8 of | 100 | NA | 94.5 | 0.14 | 71.3 | 11.50 |
| WO2019/119067 | | | | | | |
| Compound 4 of | 100 | NA | 92.6 | 0.41 | 70.3 | 4.80 |
| WO2020/257870 | | | | | | |
| B12 | 100 | NA | 98.9 | 1.60 | 94.3 | 0.36 |
| B4 | 100 | NA | 102.9 | 1.15 | 92.9 | 0.44 |

The greatest differentiation in compound stability was observed at the 28-day time point (Table 18). Too little degradation had occurred at Day 9 for any clear distinctions between compounds to be observed. On the basis of the 28-day data in Table 18, compounds B4 and 1B12 are the most stable compounds.

### pH Results

The formulation pH data recorded at each time point are compiled in Table 19. There was a general trend for the pH to increase with time but the effect was not pronounced. This indicates that gross degradation had not occurred.

TABLE 19
pH results at 0, 9 and 28 days

| Compound | 0-days | 9-days | 28-days |
|---|---|---|---|
| A107 | 5.5 | 5.6 | 5.7 |
| A204 | 5.0 | 5.2 | 5.2 |
| B12 | 5.6 | 5.7 | 5.7 |
| B4 | 5.6 | 5.7 | 5.7 |

### Conclusions

These UHPLC and pH stability data suggest that all compounds of the invention possess substantial storage stability. These data show that compounds B4 and B12 demonstrate improved stability compared to other structurally related TLR2 agonists.

### Example 13

This example details the development of a spray dried formulation of compound A204 for nasal delivery, as a broad-spectrum prophylaxis against respiratory viral and bacterial infections in at risk-populations.

Several placebo development batches were produced initially through the spray drying of mannitol or trehalose with hydroxypropyl methylcellulose (HPMC). The anticipated dose of A204 is around 20 μg, so the API loading in the spray-dried formulations was initially targeted as 1% w/w. Spray dried powders produced were blended with a suitable carrier (Pearlitol 50C).

### 13.1 Methods

### 13.1.1 Processing Methods

### 13.1.1.1 Feed Solution Preparation

All spray drying feed solutions were prepared in deionised (DI) water. The A204 (sometimes referred to in this example as “API”) was shown to be fully soluble in deionised (DI) water at a concentration of up to 2 mg/mL. All other excipient components (mannitol, trehalose and HPMC) were also shown readily soluble in the DI water.

For spray drying feed solutions where Hydroxypropyl methylcellulose (HPMC) was included in the formulation, the polymer component was first added, under stirring, to heated (˜50-60° C.) DI water. This enabled a better dispersion of the HPMC. The mixture was then quickly allowed to return to an ambient (˜20° C.) temperature to complete the dissolution of the HPMC solids. Where HPMC was not included in the formulation, excipient components (e.g., mannitol or trehalose) were added to ambient DI water.

Once the excipient components had fully dissolved, the API was added, under stirring, until fully dissolved. The solution was spray dried immediately after the addition of the API.

### 13.1.1.2 Spray Drying

All feed solutions were spray dried on a ProCepT 4M8-Trix spray dryer, fitted with an ultrasonic nozzle and set to operate at a frequency of 25 kHz.

For the majority of formulations spray dried, a target outlet temperature of 80° C. and liquid feed rate between 1.5-2.0 g/min was used. The liquid feed rate did fluctuate throughout the spray drying process, primarily as a result of the ultrasonic nozzle, thus the range of feed rate was recorded.

In all cases, the spray dried material was collected directly into amber glass vials which were sealed with parafilm, to prevent moisture ingress, and stored at 2-8° C.

Any subsequent powder handling was performed within a reduced humidity cabinet at ambient lab temperatures (20-25% RH and 18-22° C.).

### 13.1.1.3 Blending

Blends of spray dried powder and a mannitol diluent were prepared. The API loading within the blended mixture was up to 100 times lower than the spray dried powder (from 1% w/w down to 0.01% w/w).

Blends were prepared using two different mixing techniques; firstly using a single-sandwich method, where half the mannitol would be placed into the mixing container. This was followed by the whole amount of spray dried powder and the remaining half of the mannitol on top. The second mixing method was a doubling up method, where the spray dried powder was initially combined with an equal amount of mannitol diluent. This would be followed by incorporating ever increasing volumes of the mannitol diluent (equal to the mixed amount) until all the required mannitol was added.

For both methods, the combined powder mixtures were blended using a Tubular T2C low shear mixer set to 48 rpm.

### 13.1.2 Analytical Methods

The methods summarised in this section were used throughout the work detailed in this example. They include testing of the liquid feed solutions as well as the resulting spray dried powders.

### 13.1.2.1 Viscosity Measurements of Feed Solutions

The viscosity measurements of feed solutions were measured using a Brookfield DV-III+Rheometer. A CP-40 cone spindle was used for the measurements (0.8° cone angle) and the rheometer was calibrated using a 21.05 cP reference standard at 25° C., prior to commencing sample measurements. Samples were pipetted (0.5 mL, P1000 pipette) into the centre of the cup and analysed in triplicate at 10 rpm.

### 13.1.2.2 pH Measurements of Feed Solutions

The pH of the feed solutions was measured using a Mettler Toledo Seven Compact pH meter.

### 13.1.2.3 High Performance Liquid Chromatography (HPLC)

The content and impurity profile of A204 within the starting feed solutions, spray dried powders and blends was determined using an Agilent 1200 Series HPLC instrument. Details of the HPLC method used are shown in Table 20.

TABLE 20
A204 HPLC Assay Method

| | A204 HPLC Method | | |
|---|---|---|---|
| Column | Phenomenex Kinetex Biphenyl, 50 × | | |
| | 2.1 mm, 2.6 μm | | |
| Column Temperature | 40° C. | | |
| Detection | UV (205 nm) | | |
| Auto Sampler Temperature | 20° C. | | |
| Wash Vial | Water:Methanol (1:1) | | |
| Run Time | 10 min | | |
| Flow Rate | 0.5 mL/min | | |
| Mobile Phase A | 5 mM Ammonium Formate | | |
| Mobile Phase B | Acetonitrile | | |
| Gradient | Time | Mobile | Mobile |
| | (min) | Phase A (%) | Phase B (%) |
| | 0 | 55 | 45 |
| | 0.1 | 55 | 45 |
| | 8.0 | 25 | 75 |
| | 8.5 | 25 | 75 |
| | 8.6 | 55 | 45 |

| | |
|---|---|
| Injection Volume | 5 μL (increased to 50 μl) |
| Sample Diluent | Deionised water |

### 13.1.2.4 Residual Moisture Content (RMC) Determination by Loss on Drying (LOD)

Residual moisture content (RMC) within spray dried powders was determined by loss on drying (LOD). Analysis was performed using a TA Instruments Q5000 SA Dynamic Vapour Sorption (DVS) analyser. For each measurement approximately 15 to 20 mg of powder was loaded into a quartz glass sample crucible. The sample was measured using the following method:
1: Equilibrate at 25.00° C.
2: Humidity 0.00%
3: Ramp 1.00° C./min to 60.00° C.
4: Abort next iso if weight (%)<0.0100 for 5.00 min
5: Isothermal for 240.00 min
6: End of method

The residual moisture was calculated as a percentage weight loss on drying.

### 13.1.2.5 Dry Powder Particle Sizing Analysis by Sympatec

Laser particle size analysis of spray dried powders was performed using a Sympatec HELOS particle size analyser with an ASPIROS disperser, fitted with an R3 lens (0.5-175.0 μm range).

Approximately ˜50 mg powder was used for each measurement. Dispersal was achieved using compressed air at a pressure of 1 bar.

### 13.1.2.6 Differential Scanning Calorimetry (DSC)

DSC analysis was undertaken using a TA instruments Q20 MDSC with an autosampler and refrigerated cooling accessory. Approximately 1-5 mg of sample was sealed in a T Zero aluminium pan using a T Zero pan press. Samples were analysed under an N₂ flow (50 mL/min) and data analysis was undertaken using TA Instrument Universal Analysis 2000 software (build 4.5.0.5).

The samples were analysed using a standard DSC method outlined below.

### 13.1.2.7 Standard DSC Method

1: Data Storage: Off
2: Equilibrate at −20° C.
3: Isothermal for 5.00 min
4: Data Storage: On
5: Ramp 10.00° C./min to 300.00° C.

### 13.1.2.8 Differential Vapour Sorption (DVS)

DVS analysis was performed using a TA Instruments Q5000 SA Dynamic Vapour Sorption (DVS) analyser. For each measurement approximately 10 mg of powder was loaded into a quartz glass sample crucible. Powered samples were subject to initial desorption from ambient to 0% RH, followed by adsorption up to 90% RH and subsequent desorption to 0% RH. The method of analysis is outlined below:
1: Equilibrate at 25.00° C.
2: Humidity 0.00%
3: Abort next iso if weight (%)<0.01 for 5 min
4: Isothermal for 240 min
5: Abort next iso if weight (%)<0.01 for 5 min
6: Step humidity 10% every 240 min to 90%
7: Abort next iso if weight (%)<0.01 for 5 min
8: Step humidity 10% every 240 min to 0%
9: End of humidity

### 13.1.2.9 Scanning Electron Microscopy (SEM)

SEM images were obtained using a FEI Quanta650 ESEM (JEOL 6490LV SEM). The accelerating voltage was set at 5 kV with a working distance of 10.00 mm. SEM images were taken at a magnification of ×100, ×250, ×500, ×1000, ×2000 and ×4000. To prepare samples, powder was scattered onto an adhesive carbon tab on an aluminium SEM stub. Excess powder was removed using a compressed air duster, and the sample stubs were platinum coated in a Polaron SC7640 sputter coater for 90 s at ˜2.2 kv, 15 mA (approximately 12-15 nm). Samples were then transferred to the SEM for image analysis where they were imaged in high vacuum mode.

### 13.2 Accelerated Stability Study

### 13.2.1 Stability Study Set Up

The spray dried powders and blends were aliquoted under reduced humidity into 4 mL amber vials and 30 mL amber jars, respectively.

The containers were sealed using Parafilm before being heat-sealed into foil laminate pouches with desiccant.

The sample pull points and storage conditions for the spray dried powder samples are shown in Table 21 and Table 22 below. At each timepoint, samples were evaluated by DSC, Residual Moisture Content (by LOD), PSD and HPLC assay.

TABLE 21
Stability schedule for spray dried powders

| Storage condition | T = 0 | T = 7 d | T = 3 w | T = 6 w | Spare |
|---|---|---|---|---|---|
| 5° C. | N/A | | | | x |
| 25° C./60% RH | | x | x | x | x |
| 40° C./75% RH | | x | x | x | x |

For blended powders, samples were analysed by HPLC assay and Residual Moisture Content (by LOD) at the sample pull points detailed in Table 22:

TABLE 22
Stability schedule for blended powders

| Storage condition | T = 0 | T = 3 w | T = 6 w | Spare |
|---|---|---|---|---|
| 5° C. | N/A | | | x |
| 25° C./60% RH | | x | x | |
| 40° C./75% RH | | x | x | |

### 13.3 Results

13.3.1 Incorporation of A204 into Spray Dried Formulations

### Feed Solution Preparation & Spray Drying

A204 was added in the formulation at either a 1% or 5% w/w loading. The compositions of batches with the incorporation of A204, the spray drying parameters and processing yields are detailed in Table 23 below. The processing parameters are excipient mix were selected based on pre-trial screening data optimised to provide particles of an X₅₀ of 20-30 μm and <10% of particles below 10 μm. This particle size distribution is preferred for nasal drug delivery of A204.

TABLE 23
Formulation details and spray drying - A204 batches

| Batch | | Solids | pH of | Spray Drying | | |
|---|---|---|---|---|---|---|
| Number/ | | content | Feed | Feed Rate | Outlet Temp | Yield |
| Size | Formulation Details | (% w/v) | Solution | (g/min) | (° C.) | (%) |
| 163#008A-01 | A204:Mannitol:HPMC | 7.5 | 5.9 | 1.4-1.9 | 81-86 | 86.0 |
| 3.75 g | (1:69:30 w/w/w) | | | | | |
| 163#012A-01 | A204:Trehalose:HPMC | 7.5 | 7.3 | | 80-85 | 88.6 |
| 3.75 g | (1:69:30 w/w/w) | | | | | |
| 163#012B-01 | A204:Mannitol:HPMC | 7.5 | 6.2 | | 64-69 | 70.9 |
| 3.88 | (1:67:32 w/w/w) | | | | | |
| 163#020A-01 | A204:Mannitol:HPMC | 7.5 | 5.5 | | 74-85 | 78.0 |
| 1.125 g | (5:65:30 w/w/w) | | | | | |

All feed solutions spray dried to produce a white powder with processing yields ranging from 71% up to 89%.

When the outlet spray drying temperature was reduced, the percentage yield was lower (for batch 163 #012B-01). This again was primarily as a result inefficient droplet drying and product loss to the spray dryer glassware.

The percentage yield for batch 163 #020A-01, where the A204 content was increased to 5% w/w, was also marginally lower in comparison to batches spray dried with a 1% w/w A204 content. However, this is more likely to be due to the comparatively smaller batch size on this occasion.

### 13.3.2 Particle Size Analysis

Particle size analysis of A204 incorporated spray dried powders was carried out and data is summarised below in Table 24.

TABLE 24
Particle size distribution

| | | %<5 | %<10.5 | X50 | X90 | VMD |
|---|---|---|---|---|---|---|
| Batch | Formulation | μm | μm | (μm) | (μm) | (μm) |
| 163#008A-01 | A204:Mannitol:HPMC | 1.75 | 2.29 | 29.49 | 43.95 | 30.56 |
| | (1:69:30 w/w/w) | | | | | |
| 163#012A-01 | A204:Trehalose:HPMC | 1.42 | 2.09 | 30.04 | 44.46 | 30.98 |
| | (1:69:30 w/w/w) | | | | | |
| 163#012B-01 | A204:Mannitol:HPMC | 1.38 | 1.92 | 29.19 | 41.70 | 29.90 |
| | (1:67:32 w/w/w) | | | | | |
| 163#020A-01 | A204:Mannitol:HPMC | 1.87 | 2.38 | 30.74 | 44.84 | 31.21 |
| | (5:65:30 w/w/w) | | | | | |

For all batches produced, a broadly comparable particle size distribution was measured with all batches falling within the defined specification for nasal drug delivery (≤10% below 10 μm).

Increasing the API content, for batch 163 #020A-01, up to 5% w/w was shown not to influence the overall particle size distribution of the spray dried powder.

### 13.3.3 Residual Moisture Content (RMC) Determination by Loss on Drying (LOD)

The residual moisture content of the A204 incorporated spray dried batches is summarised below in Table 25.

TABLE 25
Residual moisture content by loss on drying

| | | | Residual |
|---|---|---|---|
| | | Outlet | Moisture |
| Batch | Formulation | Temp (° C.) | Content (%) |
| 163#008A-01 | A204:Mannitol:HPMC | 81-86 | 1.03 |
| | (1:69:30 w/w/w) | | |
| 163#012A-01 | A204:Trehalose:HPMC | 80-85 | 2.58 |
| | (1:69:30 w/w/w) | | |
| 163#012B-01 | A204:Mannitol:HPMC | 64-69 | 1.30 |
| | (1:67:32 w/w/w) | | |
| 163#020A-01 | A204:Mannitol:HPMC | 74-85 | 1.27 |
| | (5:65:30 w/w/w) | | |

The residual moisture content was notably lower in the mannitol-based formulations compared to the batch prepared with trehalose. The residual moisture content was shown to increase marginally in the mannitol-based formulations when the spray drying outlet temperature was decreased from ˜83° C. down to −65° C. or when the API loading was increased from 1% w/w up to 5% w/w.

### 13.3.4 A204 Content by HPLC Analysis

The A204 content within the powders was assayed using the HPLC method to determine the actual A204 loading (vs. the theoretical loading). A summary of assay data is shown below in Table 26.

TABLE 26
Residual moisture content by loss on drying

| | | % Assay (vs. |
|---|---|---|
| Batch | Formulation | theoretical loading) |
| 163#008A-01 | A204:Mannitol:HPMC | 93.6 (n = 2) |
| | (1:69:30 w/w/w) | |
| 163#012A-01 | A204:Trehalose:HPMC | 97.5 (n = 5) |
| | (1:69:30 w/w/w) | |
| 163#012B-01 | A204:Mannitol:HPMC | 92.6 (n = 5) |
| | (1:67:32 w/w/w) | |
| 163#020A-01 | A204:Mannitol:HPMC | 101.7 (n = 2) |
| | (5:65:30 w/w/w) | |

HPLC analysis showed that the A204 was broadly retained within all spray dried powders, with percentage assay values of 93-102% of nominal loadings.

13.4 Blending of Active API Spray Dried Powders with a Mannitol Diluent

Blends with a lower A204 loading were produced by combining the spray dried powder described in this example with the Pearlitol 50C mannitol diluent.

The spray dried powders containing A204 at a 1% w/w loading were combined with mannitol at a 1:99 ratio, to produce blends with a 0.01% w/w A204 loading.

Blends were prepared using the doubling up technique and were blended using a Turbula T2C low shear mixer operating at 48 rpm for 15 minutes.

Table 27 below summarises the content uniformity of A204 within the blended powders prepared.

TABLE 27
Content Uniformity of blended spray dried powders with mannitol

| | | Mixing | | Mean % Assay | |
|---|---|---|---|---|---|
| | Spray Dried | Duration | Mixing | (vs. theoretical | RSD |
| Batch Number | Component:Mannitol Ratio | (min) | Technique | loading, n = 10) | (%) |
| 163#013A-01 | A204:Mannitol:HPMC | 15 | Doubling | 113.1 | 8.2 |
| | (163#008A-01):Mannitol | | Up | | |
| | (50C) (1:99) | | Method | | |
| 163#013C-01 | A204:Trehalose:HPMC | 15 | | 109.6 | 3.5 |
| | (163#012A-01):Mannitol | | | | |
| | (50C) (1:99) | | | | |

Results from these blended batches showed that the spray dried powder containing the active API, was successfully blended with a mannitol diluent to produce a relatively homogenous final mixture.

### Residual Moisture Content (RMC) Determination by Loss on Drying (LOD)

The residual moisture content of both blended batches is summarised below in Table 28.

TABLE 28
Residual moisture content by loss on drying for blended powders

| | | Residual Moisture |
|---|---|---|
| Batch | Formulation | Content (%) |
| 163#013A-01 | A204:Mannitol:HPMC | 0.00* |
| | (163#008A-01):Mannitol | |
| | (50C) (1:99) | |
| 163#013C-01 | A204:Trehalose:HPMC | 0.02 |
| | (163#012A-01):Mannitol | |
| | (50C) (1:99) | |

The residual moisture content was practically zero for both blended batches, with batch 163 #013A-01 actually gaining mass (0.03%) over the loss on drying analysis run.

### 13.5 Accelerated Stability Study

The spray dried powders (163 #008A-01, 163 #012A-01 and 163 #020A-01) and blends (163 #013A-01 and 163 #013C-01) were placed on stability at accelerated storage conditions.

### 13.5.1 A204 Content & Related Substances of Spray Dried Powders by HPLC Analysis

The A204 content within the spray dried powders was assayed after storage at 25° C./60% RH and 40° C./75% RH for 7 days, 3 and 6 weeks. A summary of assay data is shown below in Table 29.

TABLE 28
A204 content analysis by HPLC - Accelerated stability study for spray dried powders

| | | % Assay (vs. theoretical loading) | | | | |
|---|---|---|---|---|---|---|
| Batch | Formulation | Storage | T = 0 | T = 7 days | T = 3 weeks | T = 6 weeks |
| 163#008A-01 | A204:Mannitol: HPMC | T = 0 | 93.6 | — | — | — |
| | (1:69:30 w/w/w) | | (n = 2) | | | |
| | | 25° C./60% RH | — | 94.4 | 93.4 | 86.4 |
| | | 40° C./75% RH | — | 87.5 | 81.8 | 70.5 |
| 163#012A-01 | A204:Trehalose:HPMC | T = 0 | 97.5 | — | — | — |
| | (1:69:30 w/w/w) | | (n = 5) | | | |
| | | 25° C./60% RH | — | 91.8 | 93.5 | 82.5 |
| | | 40° C./75% RH | — | 85.8 | 78.4 | 63.6 |
| 163#020A-01 | A204:Mannitol: HPMC | T = 0 | 101.7 | — | — | — |
| | (5:65:30 w/w/w) | | (n = 2) | | | |
| | | 2-8° C. | | — | 99.8 | — |
| | | | | | (T = 4 wks) | |
| | | 25° C./60% RH | — | — | 77.1 | — |
| | | 40° C./75% RH | — | — | 42.0 | — |

In all cases, there was a notable decrease in the A204 content within the spray dried powders when stored at accelerated storage conditions. The decline in A204 content was greatest at the 40° C./75% RH storage condition, within powders prepared with trehalose/HPMC and in the batch prepared at the higher 5% w/w API loading (163 #020A-01).

### 13.5.2 A204 Content of Blended Powders by HPLC Analysis

The A204 content within the blended powders was assayed after storage at 25° C./60% RH and 40° C./75% RH for 3 and 6 weeks. A summary of assay data is shown below in Table 29.

TABLE 29
A204 content analysis by HPLC - Accelerated stability study for blended powders

| | | % Assay | | | |
|---|---|---|---|---|---|
| | | (vs. theoretical loading) | | | |
| | | | | T = | T = |
| Batch | Formulation | Storage | T = 0 | 3 weeks | 6 weeks |
| 163#013A-01 | A204:Mannitol:HPMC | T = 0 | 111.9 | — | — |
| | (163#008A-01):Mannitol | 25° C./60% RH | — | 57.9 | 45.9 |
| | (50C) (1:99) | 40° C./75% RH | — | 51.1 | 31.7 |
| 163#013C-01 | A204:Trehalose:HPMC | T = 0 | 99.6 | — | — |
| | (163#012A-01):Mannitol | 25° C./60% RH | — | 51.4 | 38.7 |
| | (50C) (1:99) | 40° C./75% RH | — | 36.7 | 31.8 |

The A204 content of the blended powders saw a very significant decrease at the 3 week testing time point, which continued to decrease at the 6 week time point also. This data implied that the stability of the compound of the invention was heavily compromised by being in a low API loaded blend with the mannitol diluent.

### 13.5.3 Residual Moisture Content (RMC) Determination by Loss on Drying (LOD)

The residual moisture content of the A204 incorporated spray dried batches and the blended powders stored at accelerated stability conditions is summarised below in Table 30.

TABLE 30
Residual moisture content by loss on drying - Accelerated
stability study for spray dried and blended powders

| | | % Residual Moisture (by LOD) | | | | |
|---|---|---|---|---|---|---|
| Batch | Formulation | Storage | T = 0 | T = 7 days | T = 3 weeks | T = 6 weeks |
| 163#008A-01 | A204:Mannitol:HPMC | T = 0 | 1.03 | — | — | — |
| | (1:69:30 w/w/w) | 25° C./60% RH | — | 0.65 | 0.81 | 0.86 |
| | | 40° C./75% RH | — | 0.60 | 0.83 | 0.65 |
| 163#012A-01 | A204:Trehalose:HPMC | T = 0 | 2.58 | — | — | — |
| | (1:69:30 w/w/w) | 25° C./60% RH | — | 2.10 | 2.14 | 2.76 |
| | | 40° C./75% RH | — | 2.03 | 2.61 | 4.07 |
| 163#020A-01 | A204:Mannitol:HPMC | T = 0 | 1.27 | — | — | — |
| | (5:65:30 w/w/w) | 25° C./60% RH | — | — | 0.72 | — |
| | | 40° C./75% RH | — | — | 0.48 | — |
| 163#013A-01 | A204:Mannitol:HPMC | T = 0 | 0.00 | — | — | — |
| | (163#008A- | 25° C./60% RH | — | — | 0.00 | 0.075 |
| | 01):Mannitol (50C) | 40° C./75% RH | — | — | 0.00 | 0.00 |
| | (1:99) | | | | | |
| 163#013C-01 | A204:Trehalose:HPMC | T = 0 | 0.02 | — | — | — |
| | (163#012A- | | | | | |
| | 01):Mannitol (50C) | 25° C./60% RH | — | — | 0.11, 0.13 | 0.11 |
| | (1:99) | 40° C./75% RH | — | — | 0.04 | 0.04 |

For batches 163 #008A-01 and 163 #020A-01, prepared with mannitol, HPMC and A204 at 1% w/w and 5% w/w loadings respectively, the residual moisture content remained relatively consistent after 6 and 3 weeks at both storage conditions. The slight downward trend in residual moisture content with storage duration suggests that the desiccant sachet added to the stored vials may have had a drying effect on the powdered formulations.

For batch 163 #012-01, prepared with trehalose, HPMC and A204 at 1% w/w loading, there was an increase in the residual moisture content of the sample stored at 40° C./75% RH after 6 weeks (cf. 25° C./60% RH). This may account for some of the additional API losses observed for this particular formulation.

For both blended powders, there was negligible residual moisture, which was shown not change with storage duration. This suggested that the large API losses incurred in the blended powder was not associated with any moisture uptake on storage.

### 13.5.4 Particle Size Analysis—Spray Dried Powders

Particle size analysis of spray dried powders stored at accelerated storage conditions was carried out and results are shown in Table 31.

TABLE 31
Summary of particle size distribution measurements for spray dried and blended batches

| | | | %<5 | %<10.5 | X50 | X90 | VMD |
|---|---|---|---|---|---|---|---|
| Batch | Formulation | Storage | μm | μm | (μm) | (μm) | (μm) |
| 163#008A-01 | A204:Mannitol:HPMC | T = 0 | 1.75 | 2.29 | 29.49 | 43.95 | 30.56 |
| | (1:69:30 w/w/w) | T = 7 days | 1.44 | 2.01 | 29.66 | 43.69 | 30.45 |
| | | 25° C./60% RH | | | | | |
| | | T = 7 days | 2.25 | 2.65 | 31.16 | 43.25 | 31.07 |
| | | 40° C./75% RH | | | | | |
| | | T = 3 weeks | 1.57 | 1.62 | 28.10 | 39.73 | 28.42 |
| | | 25° C./60% RH | | | | | |
| | | T = 3 weeks | 1.84 | 2.03 | 27.87 | 38.37 | 27.88 |
| | | 40° C./75% RH (n = 2) | | | | | |
| | | T = 6 weeks | 1.56 | 1.99 | 29.31 | 42.11 | 29.76 |
| | | 25° C./60% RH (n = 1) | | | | | |
| | | T = 6 weeks | 1.23 | 1.62 | 29.99 | 44.92 | 31.07 |
| | | 40° C./75% RH (n = 2) | | | | | |
| 163#012A-01 | A204:Trehalose:HPMC | T = 0 | 1.42 | 2.09 | 30.04 | 44.46 | 30.98 |
| | (1:69:30 w/w/w) | T = 7 days | 7.42 | 7.96 | 28.53 | 39.32 | 27.22 |
| | | 25° C./60% RH | | | | | |
| | | T = 7 days | 3.70 | 4.24 | 28.61 | 40.29 | 28.37 |
| | | 40° C./75% RH | | | | | |
| | | T = 3 weeks | 1.48 | 2.19 | 28.76 | 41.81 | 29.44 |
| | | 25° C./60% RH | | | | | |
| | | T = 3 weeks | 1.84 | 2.47 | 29.22 | 41.39 | 29.42 |
| | | 40° C./75% RH | | | | | |
| | | T = 6 weeks | 2.44 | 3.05 | 30.52 | 45.20 | 31.05 |
| | | 25° C./60% RH (n = 2) | | | | | |
| | | T = 6 weeks | 1.40 | 2.12 | 29.81 | 42.97 | 30.46 |
| | | 40° C./75% RH | | | | | |
| 163#020A-01 | A204:Mannitol:HPMC | T = 0 | 1.87 | 2.38 | 30.74 | 44.84 | 31.21 |
| | (5:65:30 w/w/w) | T = 3 weeks | 1.28 | 1.80 | 30.09 | 43.08 | 30.75 |
| | | 25° C./60% RH (n = 2) | | | | | |
| | | T = 3 weeks | 1.34 | 1.78 | 28.72 | 40.45 | 29.01 |
| | | 40° C./75% RH (n = 1) | | | | | |

Particle size data showed that for batch 163 #008A-01, prepared with mannitol, the size distribution remained largely unchanged at both storage conditions over the 6-week accelerated stability study. During testing at the 3- and 6-week time points, the particle sizer software did indicate the presence of course particles (outside of the measuring range of the lens) in the powders and on a number of occasions this resulted in null and void data, and consequently only single or duplicate measurements were possible.

The particle size data for batch 163 #012A-01, prepared with trehalose, showed that the size distribution remained largely unchanged at both storage conditions over the 6-week accelerated stability study.

As observed for both previous batches, the particle size data for batch 163 #020A-01, prepared with mannitol with 5% w/w A204, showed that the size distribution again remained unchanged at both storage conditions over the 3-week storage period.

### 13.6 Device Filling

The spray dried formulations were filled into Aptar Unidose nasal devices as detailed below in Table 32.

TABLE 32
Aptar Unidose Device Filling

| | Fill | API | Number |
|---|---|---|---|
| Formulation | Weight (mg) | Dose (μg) | of Devices |
| A204:Mannitol:HPMC | 10 mg | 100 μg | 10 |
| (1:69:30 w/w/w) | 30 mg | 300 μg | 10 |
| A204:Mannitol:HPMC | 25 mg | 1000 μg | 10 |
| (5:65:30 w/w/w) | | | |

All devices were filled within a reduced humidity cabinet (˜20% RH) and individually heat sealed within a foil laminate pouches with a desiccant sachet.

### 13.7 Conclusions

This example details development of spray dried formulations of A204. The above data show that A204 can effectively be spray dried. However, in order to prepare formulations comprising the very low concentration of A204 required (due to its extreme potency), blended formulations of the spray dried powders with a mannitol diluent were required. The A204 in the spray-dried and the blended formulations suffered unacceptable stability losses under accelerated conditions.

### Example 14—Excipient Compatibility

In view of the instability of A204 identified above, an excipient compatibility study was undertaken and detailed in this example.

The formulations prepared in Example 13 all contain the A204, HPMC and either mannitol or trehalose. The solids components are dissolved in deionised water and spray dried on a ProCepT 4M8-Trix, fitted with an ultrasonic nozzle. Powdered material has been placed on storage at the accelerated storage conditions and observed significant decreases in A204 content.

This example describes the results of combinations of A204 with potential excipients outlined in Table 33.

TABLE 33
Excipients to be screened with A204

| Sample | Supplier |
|---|---|
| Hydroxypropyl-β-Cyclodextrin (HP-β-CD), Parental | Roquette |
| β-Cyclodextrin (β-CD) | Roquette |
| Mannitol (Pearlitol Pyrogen Free PF) | Barentz |
| Erythritol (Zerose) | Cargill |
| Xylitol (Xylisorb 90) | Barentz |
| Sorbitol (Neosorb P100T) | Barentz |
| Myo-Inositol | Merck |
| Adonitol | Sigma |
| Pectin (Unipectine RougePH 320 NH) | Cargill |
| Hydroxypropyl methylcellulose (HPMC -PharmaCoat 606) | Shinetsu |
| Hydroxypropyl cellulose(HPC - Klucel EXF) | Ashland |
| Hydroxyethyl cellulose(HEC- Natrosol 250HX) | Ashland |
| Methylcellulose (MC) | Alfa Aesar |

Formulations comprising A204 alone (2 mg/mi) and combined as a solution of A204 (2 mg/mi) with an equivalent volume of various combinations of excipient component(s) detailed in Table 33 into a single solution. All test solutions where then freeze dried according to the protocol set out below. The freeze dried samples were then stored at 25° C./60% RH and 40° C./75% RH for 3 weeks. Following storage, the formulations were be assayed by HPLC according to the protocol described in Example 13 for A204 content and impurities.

### Protocol for Preparation of Samples

Within a volumetric flask a 2 mg/ml A204 solution in water was prepared.

All other excipient components were prepared at 10 mg/ml in water.

The appropriate volumes of API and excipient(s) were pipetted into 10 mL glass vials, suitable for freeze drying. The vials were stoppered and gently swirled. Except where otherwise noted, the ratio of A204 to each excipient included in the solution was 1:5 w/w A204:excipient.

The rubber stoppers were loosened and vials placed at −40° C. (within freeze drier) and allowed to freeze for a minimum of 4 hours.

Vials were placed under vacuum, allowed to stabilise before initialising the following freeze drying parameters—
Ramp from −40° C. up to +20° C. at 2° C./hour.
Hold for 24 hours (minimum).

At the end of the freeze drying cycle, the chamber was purged with nitrogen and moved down the shelf so that the rubber stoppers close down on the vials. The vials were removed from the freeze drier. Vials were then capped and crimped.

All vials placed within foil laminate pouch(es) with desiccant sachets in the pouch and heat sealed.

Pouch placed at 40° C. for 3 weeks. Sample 1 vials (A204 only) at 2-8° C. as well and used as a reference sample.

The entire content of each vial was assayed for A204 content by HPLC.

### Results

Results from this excipient screen are summarised in Table 34.

TABLE 34
3 week stability results for freeze dried powders
of various combinations of A204 and excipient(s)

| | | Mean % |
|---|---|---|
| Formulation | Conditions | Recovery |
| A204 only | 2-8° C./3 weeks | 96.9 |
| A204 only | 40° C./3 weeks | 91.7 |
| A204:HP-β-CD | 40° C./3 weeks | 96.5 |
| A204:β-CD | 40° C./3 weeks | 96.4 |
| A204:mannitol (pyrogen free) | 40° C./3 weeks | 95.1 |
| A204:erythritol | 40° C./3 weeks | 92.8 |
| A204:xylitol | 40° C./3 weeks | 96.5 |
| A204:sorbitol | 40° C./3 weeks | 94.2 |
| A204:myo-inositol | 40° C./3 weeks | 96.4 |
| A204:adonitol | 40° C./3 weeks | 84.7 |
| A204:HP-β-CD:mannitol | 40° C./3 weeks | 98.3 |
| (pyrogen free) | | |
| A204:HP-β-CD:erythritol | 40° C./3 weeks | 96.2 |
| A204:HP-β-CD:xylitol | 40° C./3 weeks | 93.3 |
| A204:HP-β-CD:myo-inositol | 40° C./3 weeks | 97.0 |
| A204:HP-β-CD:adonitol | 40° C./3 weeks | 67.0 |
| A204:β-CD:mannitol (pyrogen | 40° C./3 weeks | 95.0 |
| free) | | |
| A204:β-CD:erythritol | 40° C./3 weeks | 88.7 |
| A204:β-CD:xylitol | 40° C./3 weeks | 91.6 |
| A204:β-CD:sorbitol | 40° C./3 weeks | 90.7 |
| A204:β-CD:myo-inositol | 40° C./3 weeks | 95.0 |
| A204:β-CD:adonitol | 40° C./3 weeks | 69.9 |
| A204:pectin 1:5 w/w | 40° C./3 weeks | 83.7 |
| A204:HPMC 1:5 w/w | 40° C./3 weeks | 85.3 |
| A204:HPC 1:5 w/w | 40° C./3 weeks | 93.3 |
| A204:HEC 1:1 w/w | 40° C./3 weeks | 79.4 |
| A204:MC 1:2.5 w/w | 40° C./3 weeks | 88.1 |
| A204:HP-β-CD:pectin | 40° C./3 weeks | 87.1 |
| A204:HP-β-CD:HPMC | 40° C./3 weeks | 88.7 |
| A204:HP-β-CD:HEC | 40° C./3 weeks | 79.9 |
| A204:HP-β-CD:MC | 40° C./3 weeks | 90.9 |
| A204:β-CD:pectin | 40° C./3 weeks | 88.8 |
| A204:β-CD:HPMC | 40° C./3 weeks | 95.4 |
| A204:β-CD:HPC | 40° C./3 weeks | 93.1 |
| A204:β-CD:MC | 40° C./3 weeks | 96.3 |
| A204:HP-β-CD:mannitol(pyrogen | 40° C./3 weeks | 84.5 |
| free):pectin | | |
| A204:HP-β-CD:mannitol(pyrogen | 40° C./3 weeks | 90.0 |
| free):HEC | | |
| A204:HP-β-CD:mannitol(pyrogen | 40° C./3 weeks | 95.8 |
| free):MC | | |
| A204:β-CD:mannitol(pyrogen | 40° C./3 weeks | 84.7 |
| free):pectin | | |
| A204:β-CD:mannitol(pyrogen | 40° C./3 weeks | 92.4 |
| free):HPMC | | |
| A204:β-CD:mannitol(pyrogen | 40° C./3 weeks | 95.1 |
| free):HPC | | |
| A204:β-CD:mannitol(pyrogen | 40° C./3 weeks | 81.8 |
| free):HEC | | |
| A204:β-CD:mannitol(pyrogen | 40° C./3 weeks | 96.8 |
| free):MC | | |

In the formulations described in Table 34 the inclusion of a cyclodextrin improves the stability of A204, if not relative to the A204 only formulation then relative to a formulation of an excipient absent the cyclodextrin.

### Example 15—Liquid Formulation Stability Study

Liquid formulations (F1 to F9) were prepared in 0.9% w/v NaCl, 0.1% w/v EDTA solution, with their compositions detailed in table 35. Unlike in Example 11 the samples tested in this Example did not include a buffer. All samples were filtered (0.2 μm PTFE) and aliquoted into amber HPLC insert vials for stability storage at 25° C./60% RH, 40° C./75% RH, 2-8° C. and −20° C. The samples were tested for active compound content at 3, 6 and 9 weeks.

TABLE 35

| | Composition | | Mass of β-CD in |
|---|---|---|---|
| Formulation | (ratio by | Mass of active | 5 mL volumetric |
| ID | weight) | compound (mg) | flask (mg) |
| F1 | A204 | 5 | 0 |
| F2 | A204:β-CD (1:5) | 5 | 13.30 |
| F3 | B14 | 5 | 0 |
| F4 | F14:β-CD (1:5) | 5 | 13.13 |
| F5 | B12 | 5 | 0 |
| F6 | B12:β-CD (1:5) | 5 | 13.13 |
| F7 | B16 | 5 | 0 |
| F8 | B16:β-CD (1:5) | 5 | 13.13 |
| F9 | A204:β-CD (1:1) | 5 | 2.66 |

### Results

The results for F1 and F2 at 25° C./60% RH and 40° C./75% RH are set out in Table 36.

TABLE 36

| | |
|---|---|
| 25° C./60% RH | 40° C./75% RH |

| Timepoint (wks) | F1 | F2 | F1 | F2 |
|---|---|---|---|---|
| 3 | 93.80% | 97.79% | 79.40% | 86.53% |
| 6 | 92.65% | 95.69% | 60.70% | 77.67% |
| 9 | 88.13% | 96.41% | 51.40% | 72.88% |

These results show improvement in active stability when β-CD is included in the formulation under ambient storage conditions and under accelerated storage conditions.

TABLE 37

| | |
|---|---|
| 25° C./60% RH | 40° C./75% RH |

| Timepoint (wks) | F3 | F4 | F3 | F4 |
|---|---|---|---|---|
| 3 | 100.51 | 101.08 | 97.91 | 103.63 |
| 6 | 98.30 | 98.92 | 83.99 | 94.44 |
| 9 | 93.07 | 94.14 | 57.62 | 70.31 |

Example 11 describes that B14 possesses improved stability over A204. These results show that formulation of B14 with β-CD improves storage stability under both aging conditions.

The results for F5 and F6 at 25° C./60% RH and 40° C./75% RH are set out in Table 38.

TABLE 38

| | |
|---|---|
| 25° C./60% RH | 40° C./75% RH |

| Timepoint (wks) | F5 | F6 | F5 | F6 |
|---|---|---|---|---|
| 3 | 112.28 | 110.85 | 101.60 | 102.74 |
| 6 | 109.55 | 108.18 | 101.33 | 93.70 |
| 9 | 109.74 | 108.64 | 84.90 | 89.71 |

While B12 appears to be more stable under accelerated conditions compared with A204 and B14, these results show that formulation of B12 with β-CD improves storage stability under accelerated conditions over the 9 week storage period. Stability of B12 under ambient aging conditions (25° C./60% RH) is comparable for both F5 and F6, the lack of difference appears to relate to the general stability of B14.

The results for F7 and F8 at 25° C./60% RH and 40° C./75% RH are set out in Table 39.

TABLE 39

| | |
|---|---|
| 25° C./60% RH | 40° C./75% RH |

| Timepoint (wks) | F7 | F8 | F7 | F8 |
|---|---|---|---|---|
| 3 | 96.28 | 98.33 | 89.97 | 92.67 |
| 6 | 106.89 | 109.88 | 88.35 | 98.12 |
| 9 | 106.59 | 109.21 | 70.77 | 84.75 |

While B16 appears to be more stable under accelerated conditions compared with A204 and B14, these results show that formulation of B16 with β-CD improves storage stability under both aging conditions.

### Example 16—Spray Dried Formulation of A204 12 Week Stability Study

Following a similar spray drying procedure described in Example 13, the following formulations described in Table 40 were prepared.

Batches were spray dried using a ProCepT 4M8-TriX spray dryer fitted with a large cyclone and an ultrasonic nozzle set to 25 kHz operated using the general conditions outlined in Table 40. The formulations were spray dryed from a solution comprising water as the liquid carrier.

TABLE 40

| Inlet | Outlet | | | Cyclone | Nozzle | Liquid |
|---|---|---|---|---|---|---|
| temperature | temperature | Cyclone in | Airflow in | cooling air | cooling air | feed rate |
| (° C.) | (° C.) | temperature | (m³/min) | (bar) | (bar) | (g/min) |
| ~105 | ~80-85 | ~70 | ~0.70 | 0.50 | 2.0 | ~1.8-2.0 |

TABLE 41

| | | Mean Particle Size Distribution (n = 3)* | | | | |
|---|---|---|---|---|---|---|
| Formulation | | %<5 | %<10.5 | X₅₀ | X₉₀ | VMD |
| ID | Composition (w/w) | μm | μm | (μm) | (μm) | (μm) |
| 16.1 | A204:HP-β-CD (1:99) | 5.64 | 15.30 | 22.22 | 35.81 | 22.23 |
| 16.2 | A204:HP-β-CD:mannitol | 1.80 | 3.09 | 25.50 | 35.2 | 25.72 |
| | (1:49.5:49.5) | | | | | |
| 16.3 | A204:β- | 8.49 | 24.41 | 16.06 | 26.66 | 16.06 |
| | CD:mannitol:methylcellulose | | | | | |
| | (MC) (1:20:69:10) | | | | | |
| 16.4 | A204:β-CD:myo-inositol:MC | 5.40 | 18.88 | 16.00 | 24.41 | 16.06 |
| | (1:20:69:10) | | | | | |
| 16.5 | A204:β- | 1.71 | 2.93 | 22.63 | 33.07 | 23.13 |
| | CD:mannitol:hydroxypropylcellulose | | | | | |
| | (HPC) (1:20:69:10) | | | | | |

Notes:
*particle size measured on preparation (t = 0)

Formulations 16.2 and 16.5 possess particle sizes suitable for nasal administration (due to low proportion of particles under 10 μm) without further optimisation. Formulations 16.1, 16.3 and 16.4 were suitable for assessing stability and further optimisation of spray drying conditions may provide suitable particle size distributions for nasal administration.

Aliquots of each formulations of Table 41 were stored concurrently at (i) 2-84° C., (ii) 30° C. and 65% RH (30/65) and (iii) 40° C. and 75% RH (40/75). Each aliquot was assayed at 0 weeks, 4 weeks, 8 weeks and 12 weeks storage under each condition, with results shown in Tables 42 and 43.

TABLE 42
Loss on drying (DVS) of formulations 16.1, 16.2, 16.3, 16.4
and 16.5 under various storage conditions over 12 weeks

| | | | | T = 4 | | |
|---|---|---|---|---|---|---|
| Formulation | | | | weeks | | |
| ID | Composition (w/w) | Storage | T = 0 | (w) | T = 8 w | T = 12 w |
| 16.1 | A204:HP-β-CD | 2-8° C. | 2.49 | — | — | 2.90 |
| | (1:99) | 30° C./65% RH | | 3.71 | 4.36 | 4.24 |
| | | 40° C./75% RH | | 4.20 | 3.31 | 4.25 |
| 16.2 | A204:HP-β-CD:mannitol | 2-8° C. | 0.82 | — | — | 2.80 |
| | (1:49.5:49.5) | 30° C./65% RH | | 2.48 | 2.25 | 2.34 |
| | | 40° C./75% RH | | 2.39 | 2.22 | 2.15 |
| 16.3 | A204:β- | 2-8° C. | 1.00 | — | — | 1.64 |
| | CD:mannitol:methylcellulose (MC) | 30° C./65% RH | | 1.59 | 1.34 | 1.41 |
| | (1:20:69:10) | 40° C./75% RH | | 1.45 | 1.52 | 1.40 |
| 16.4 | A204:β-CD:myo-inositol:MC | 2-8° C. | 0.63 | — | — | 1.40 |
| | (1:20:69:10) | 30° C./65% RH | | 1.21 | 1.53 | 2.29 |
| | | 40° C./75% RH | | 1.06 | 0.95 | 1.39 |
| 16.5 | A204:β- | 2-8° C. | 0.84 | — | — | 1.62 |
| | CD:mannitol:hydroxypropylcellulose | 30° C./65% RH | | 1.73 | 1.50 | 1.56 |
| | (HPC) (1:20:69:10) | 40° C./75% RH | | 1.52 | 1.43 | 1.42 |

Residual moisture contents of all formulations increased over the course of the experiment, with largest increase in the first 4 week period. The introduction of water to the formulations may have been due to the aliquoting process.

TABLE 43
A204 purity of formulations 16.1, 16.2, 16.3, 16.4 and 16.5 under various storage conditions over 12 weeks

| | | | | T = 4 | | |
|---|---|---|---|---|---|---|
| Formulation | | | | weeks | | |
| ID | Composition (w/w) | Storage | T = 0 | (w) | T = 8 w | T = 12 w |
| 16.1 | A204:HP-β-CD (1:99) | 2-8° C. | 96.72 ± | — | — | 95.28 ± |
| | | | 0.24 | | | 0.41 |
| | | 30° C./65% RH | | 92.72 ± | 94.47 ± | 89.95 ± |
| | | | | 1.79 | 3.76 | 1.98 |
| | | 40° C./75% RH | | 89.90 ± | 88.63 ± | 89.63 ± |
| | | | | 0.39 | 1.08 | 0.68 |
| 16.2 | A204:HP-β-CD:mannitol | 2-8° C. | 96.44 ± | — | — | 93.70 ± |
| | (1:49.5:49.5) | | 0.80 | | | 0.38 |
| | | 30° C./65% RH | | 92.68 ± | 87.39 ± | 88.85 ± |
| | | | | 0.42 | 2.46 | 0.79 |
| | | 40° C./75% RH | | 106.27 ± | 81.81 ± | 89.16 ± |
| | | | | 3.18 | 1.35 | 0.94 |
| 16.3 | A204:β- | 2-8° C. | 90.04 ± | — | — | 82.40 ± |
| | CD:mannitol:methylcellulose | | 1.93 | | | 1.32 |
| | (MC) (1:20:69:10) | 30° C./65% RH | | 77.74 ± | 77.16 ± | 70.38 ± |
| | | | | 4.42 | 1.14 | 1.28 |
| | | 40° C./75% RH | | 89.28 ± | 71.72 ± | 67.66 ± |
| | | | | 0.78 | 1.38 | 0.37 |
| 16.4 | A204:β-CD:myo- | 2-8° C. | 80.22 ± | — | — | 78.77 ± |
| | inositol:MC | | 1.89 | | | 1.89 |
| | (1:20:69:10) | 30° C./65% RH | | 79.18 ± | 78.48 ± | 72.39 ± |
| | | | | 2.58 | 2.63 | 0.45 |
| | | 40° C./75% RH | | 80.46 ± | 62.31 ± | 58.58 ± |
| | | | | 1.02 | 0.23 | 0.62 |
| 16.5 | A204:β- | 2-8° C. | 95.90 | — | — | 92.24 ± |
| | CD:mannitol:hydroxypropylcellulose | | | | | 1.60 |
| | (HPC) (1:20:69:10) | 30° C./65% RH | | 82.52 ± | 80.12 ± | 77.59 ± |
| | | | | 1.31 | 1.48 | 0.66 |
| | | 40° C./75% RH | | 80.32 ± | 60.24 ± | 55.75 ± |
| | | | | 0.87 | 0.13 | 0.29 |

The data presented in Table 43 show that formulations 16.1 and 16.2 possess the highest stability when stored under both accelerated conditions. Formulations containing MC or HPC were less stable under all conditions.

Scanning electron microscope images of the particles of these formulations were taken at each sampling timepoint. These images suggest changes to particle surface morphology after storage, with more pronounced changes for particles stored under either 30/65 or 40/75 accelerated conditions.

### Example 17—Spray Dried Formulation of A204 4 Week Stability Study

Following a similar spray drying procedure described in Example 16, the following formulations described in Table 44 were prepared.

TABLE 44

| | | |
|---|---|---|
| 17.1 | A204:HP-β-CD:Mannitol | Fine white powder with three notably |
| | (1:74.5:24.5) | large, soft (easily broken) agglomerates. |
| | | Some material adhered to the upper vial |
| | | wall which was difficult to remove by |
| | | scraping with a spatula. |
| 17.2 | A204:HP-β-CD:Mannitol | Fine white powder with a few small, |
| | (1:49.5:49.5) | easily broken agglomerates. |
| 17.3 | A204:HP-β- | Fine white powder. Some material |
| | CD:Mannitol:L- | adhered to the upper vial wall which |
| | Leucine (1:70.5:20.5:8) | was difficult to remove by scraping with |
| | | a spatula. Some coating of vial walls. |
| 17.4 | A204:HP-β- | Fine white powder with variously sized |
| | CD:Mannitol:L- | aggregates which required a little force |
| | Leucine (1:45.5:45.5:8) | to break with a spatula. Some coating to |
| | | vial walls. No notable visual differences |
| | | between material stored at each of the |
| | | three conditions (2-8° C., 30° C./65% |
| | | RH, 40°/75% RH). |
| 17.5 | A204:HP-β- | Fine white powder with some slightly |
| | CD:Mannitol:Tri- | harder aggregates that could be broken |
| | Leucine | using a spatula (requiring a little force). |
| | (1:49.25:49.25:0.5) | Some coating to vial walls. |

Formulations 17.1 and 17.2 have the same components as formulations 16.1 and 16.2 for comparison purposes.

Aliquots of each formulations of Table 44 were prepared in a low humidity environment and stored concurrently at (i) 2-8° C., (ii) 30° C. and 65% RH (30/65) and (iii) 40° C. and 75% RH (40/75). Each aliquot was assayed at 0 weeks and 4 weeks, with results shown in Tables 45 and 46. Additional aliquots are stored for testing at 8 weeks and 12 weeks under each condition.

TABLE 45
Loss on drying (LOD) determined by TGA 550 (n =
1) for formulations 17.1, 17.2, 17.3, 17.4 and 17.5
under various storage conditions over 4 weeks

% LOD (n = 1)

| Formulation | Storage | T = 0 | T = 2 w | T = 4 w |
|---|---|---|---|---|
| 17.1 | 2-8° C. | 1.74 | — | — |
| | 30° C./65% RH | | — | — |
| | 40° C./75% RH | | 1.96 | 2.09 |
| 17.2 | 2-8° C. | 0.98 | — | — |
| | 30° C./65% RH | | — | — |
| | 40° C./75% RH | | 1.67 | 1.77 |
| 17.3 | 2-8° C. | 1.45 | — | — |
| | 30° C./65% RH | | — | — |
| | 40° C./75% RH | | 1.50 | 1.62 |
| 17.4 | 2-8° C. | 0.99 | — | 1.10 |
| | 30° C./65% RH | | — | 1.22 |
| | 40° C./75% RH | | 1.11 | 1.35 |
| 17.5 | 2-8° C. | 1.22 | — | — |
| | 30° C./65% RH | | — | — |
| | 40° C./75% RH | | 1.34 | 1.34 |

Relatively smaller increases in residual moisture were observed compared to those of example 16 (Table 41) suggesting that moisture was introduced during aliquoting.

TABLE 46
A204 purity of formulations 16.1, 16.2, 16.3, 16.4 and
16.5 under various storage conditions over 4 weeks

| | | | | |
|---|---|---|---|---|
| 17.1 | 2-8° C. | 93.18 ± 0.58 | — | — |
| | 30° C./65% RH | | — | — |
| | 40° C./75% RH | | 90.51 ± 0.27 | 90.17 ± 0.82 |
| 17.2 | 2-8° C. | 96.57 ± 0.44 | — | — |
| | 30° C./65% RH | | — | — |
| | 40° C./75% RH | | 93.33 ± 0.88 | 92.57 ± 0.64 |
| 17.3 | 2-8° C. | 97.82 ± 1.03 | — | — |
| | 30° C./65% RH | | — | — |
| | 40° C./75% RH | | 94.28 ± 0.43 | 94.22 ± 1.16 |
| 17.4 | 2-8° C. | 97.25 ± 0.12 | — | 97.09 ± 0.30 |
| | 30° C./65% RH | | — | 94.91 ± 1.98* |
| | 40° C./75% RH | | 93.96 ± 0.78 | 94.83 ± 1.78** |
| 17.5 | 2-8° C. | 96.49 ± 0.95 | — | — |
| | 30° C./65% RH | | — | — |
| | 40° C./75% RH | | 94.12 ± 0.76 | 90.94 ± 0.23 |

Notes:
*where n = 2, % A204 assay is 96.05 ± 0.02
**where n = 2, % A204 assay is 95.86 ± 0.03

TABLE 47
A204 purity of formulations 16.1, 16.2, 16.3, 16.4 and 16.5 under various
storage conditions over 4 weeks adjusted for residual moisture content

| | | Average % A204 Assay (n = 3) | | |
|---|---|---|---|---|
| Formulation | Storage | T = 0 | T = 2 w | T = 4 w |
| 17.1 | 2-8° C. | 94.83 ± 0.59 | — | — |
| | 30° C./65% RH | | — | — |
| | 40° C./75% RH | | 92.32 ± 0.28 | 92.09 ± 0.83 |
| 17.2 | 2-8° C. | 97.53 ± 0.45 | — | — |
| | 30° C./65% RH | | — | — |
| | 40° C./75% RH | | 94.92 ± 0.90 | 94.23 ± 0.65 |
| 17.3 | 2-8° C. | 99.26 ± 1.05 | — | — |
| | 30° C./65% RH | | — | — |
| | 40° C./75% RH | | 95.72 ± 0.44 | 95.76 ± 1.18 |
| 17.4 | 2-8° C. | 98.22 ± 0.13 | — | 98.16 ± 0.30 |
| | 30° C./65% RH | | — | 96.08 ± 2.00* |
| | 40° C./75% RH | | 95.02 ± 0.79 | 96.13 ± 1.80** |
| 17.5 | 2-8° C. | 97.68 ± 0.96 | — | — |
| | 30° C./65% RH | | — | — |
| | 40° C./75% RH | | 95.40 ± 0.77 | 92.18 ± 0.24 |

The data shown in Tables 46 and 47 demonstrate that leucine is tolerated. The data for formulations 17.1 and 17.2 confirm the stability data from example 16.

### Example 18—Comparison of Liquid and Dry Powder Formulations Comprising A204 Following Intranasal Administration to Cynomolgus Macaques

To ensure that the spray dried formulations are suitable for intranasal administration and that the excipients do not impede the bioavailability of the active pharmaceutical ingredient (API), the performance of the following three formulations were compared to a liquid spray solution [1 mg/mL: Saline/EDTA+A204] and a spray dried powder formulation [70:30 mannitol:HPMC] (“Dry Powder”):
A204 Spray Dried Powder Formulation, 1% A204 with 99% 70:30 mannitol:HPMC [Dry Powder & A204]
A204 Spray Dried Powder Formulation, 1% A204 with 99% hydroxypropyl beta-cyclodextrin [Dry Powder & Hydroxypropyl-β-cyclodextrin].
A204 Spray Dried Powder Formulation, 1% A204 with 99% beta-cyclodextrin [Dry Powder & β-cyclodextrin]

Spray dried powder formulations are prepared according to procedures described in Example 13.

### Summary of Results of Example 18

The three Spray Dried Powder formulations were associated with increased levels of selected cytokines and chemokines in nasal secretions at 6 hours compared to baseline and in most cases, compared to placebo formulation,
The three Spray Dried Powder formulations exhibited an elevated level of IL-6 at 6 hours, comparable to the liquid spray solution, suggesting that spray dried powder formulations with excipients, including β-cyclodextrin and hydroxypropyl-β-cyclodextrin, do not severely impact on the biological activity of A204.
As observed for the liquid spray solution, IL-10 and IFN-α2a released in serum following administration of the Spray Dried Powder formulations remain low; <4 pg/mL for IL-10 and <12 pg/mL for IFN-α2a.
Serum levels of MCP-1, MIP1-α and Gro-α were observed for all formulations. Levels of MCP-1, MIP1-α and Gro-α observed in the dry powder A204 formulations were comparable to the A204 liquid spray solution.
Serum IFN-α2a, IL-1β, IL-6, IL-10, TNF-α and IFN-γ in either one or both replicates were below the detection range or below the fit curve range. This suggests that the excipients, including β-cyclodextrin and hydroxypropyl-β-cyclodextrin in the spray dried powder formulations were comparable to the A204 liquid spray solution and did not appear to affect the bioavailability of A204.

### Protocol

After quarantine 15 animals (13 males, 2 females) were subjected to sample collection (nasal swab, nasal brushing, Nasosorption and blood draw) at baseline (7 days or more prior to TA dosing). Animals received a single dose of excipient only, liquid control spray solution or dry powder formulations (see above) on Day 1. Administration was 2 actuations, one per nare. Blood was collected at several time points post dose for analysis. Post dose nasal collections were also obtained at 6 and 24 hours post dosing. The study was non-terminal and animals were transferred to an approved protocol upon completion of study activities.

### Formulation and Device Filling

A204 liquid formulation was prepared in a bulk formulation (stored at 2-8° C.) and nasal delivery devices (Aptar Unidose Dry Powder Device) were filled each day of dosing as required based on the stability under conditions of use at 1 mg/mL, per Preparation of A204 in 0.9% Saline with 0.1% w/w EDTA.

### Sampling

Samples were taken from the subjects according to the schedule outlined in Table 48.

TABLE 48
Sampling and sampling disposition schedule

| Time Point | Blood | Nasosrbtion ® | Nasal Swab | Nasal Brushing |
|---|---|---|---|---|
| Baseline^{A} | Serum and Plasma | Both Nares^{B} | Both Nares^{B} | Both Nares^{B} |
| 0.5 Hour (±5 Minutes) | Plasma | — | — | — |
| 1 Hour (±10 Minutes) | Plasma | — | — | — |
| 6 hours post dose | Serum and Plasma | Both Nares^{B} | Both Nares^{B} | n/a |
| 24 hours post dose | Serum and Plasma | Both Nares^{B} | Both Nares^{B} | Both Nares^{B} |
| Total number of Samples | Serum - 45 | 45 | 45 | 45 |
| | Plasma - 75 | | | |
| Disposition | Serum - Cytokines | Cytokines | Cytokines | Sponsor |
| | Plasma - hold for | | | bioanalysis |
| | possible Sponsor | | | |
| | bioanalysis | | | |

^{A}Samples to be taken in the following order: 1) Nasosrbtion ®, 2) Nasal Swab, 3) Nasal Brushing
^{B}Sample to be pooled post processing

### Blood Collections and Processing

Blood collections (≥1 mL into K₂EDTA or SST tube on each occasion as indicated in Table 48. Samples were then centrifuged (1300 g, 2-8° C., ≥10 minutes), plasma or serum was separated into appropriately labeled vials within 2 hours of blood collection and plasma stored frozen (−70 to −90° C.).

### Nasal Swabs and Nasosorption® Sampling

Nasal swab samples were collected with cotton tip applicators pre-soaked in sterile saline. The swab advanced into the nasal cavity and gently agitated. After collection, the tip of each swab was cut and placed in an EppendorfSafetyLock tube (with 300 μL of PBS) and immediately frozen on dry ice prior to storage at −70 to −90° C. until processed.

Nasosorption samples were obtained and processed by following the manufacturer's instructions. Unscrew the applicator using the device handle. Carefully advance the device applicator into the nasal cavity, and place flat on the on the nasal mucosa 2 minutes (±30 seconds). Remove the device, return the applicator and screw the lid closed close. Samples were placed on wet ice after collection until processing.

Nasal brushing samples were obtained with a cytology brush. After collection, the brush was placed in the sample tube. The brush was extended and retracted (3 to 5 times) to dislodged cells. The inner wire epithelial brush was advanced and cut with the wire cutter so that it fit into the sample tube and the tube was then closed. The tubes were placed on wet ice for up to 1 hour before processing.

### Nasal Sample Processing

Nasal Swabs: Swabs were vortexed with PBS to extract the sample. Complete methods will be included in the study file. Post processing pool extracts from both nares as indicated in Table 48.

Nasosorption: Samples were extracted from nasosorption strips according to the manufacturers advice as briefly described below. Post processing pool extracts from both nares as indicated in Table 48.
Step I—Add moist synthetic absorptive matrix (SAM; removed from the handle) to Eppendorf containing buffer (100 uL, when possible use PBS+1% BSA; if not available IX PBS may be substituted) vortex for 230 seconds
Step 2—SAM is removed (Cut from the applicator with clean forceps/tools used for each sample. Tools may be cleaned with 70% ethyl alcohol between samples) and is placed in a plastic mesh insert (Corning Spin-X tubes, Catalogue number: CLS9301, with a plastic mesh without a filter are preferred; any equivalent tube may be substituted): In the event that tubes with a mesh insert are not available at the time of collections, Qiagen DNeasy spin columns can be used as long as the filter is removed—the SAM can be placed into the spin column without the filter steps 3-5 can continue.
Step 3—The insert (with SAM) is then placed back into the original tube containing buffer from Step 1
Step 4—The Eppendorf containing the mesh insert, SAM and buffer is centrifuged for 20 min at 4 C at 16,000G
Step 5—The mucosa! lining fluid (and buffer) is aliquoted into labelled cryotubes in up to 2, approximately 1 00 uL aliquots (aliquoted by micropipette, noting aliquots <100 uL) and frozen until use at −70 to −90° C.

Nasal Brushing: To release the epithelial cells from the brushes, within one hour after placing the brushes into the tube with approximately 200 μL RNALater (or equivalent) reagent, the tubes were vortexed on a low setting for 3 minutes.

Post processing extracts were pooled from both nares as indicated in Table 47. The epithelial cells in the RNALater (or equivalent) tube were stored at 2 to 8° C. overnight, after which the tubes were frozen at −70 to −90° C.

### Cytokine Analysis

Nasal swab and nasosorption samples obtained from all animals were used for cytokine analysis using the MSD platform. Cytokines analysis was performed per manufacturer kit instructions. Cytokine analysis will include: IL-6, IL-8, IL-Iβ, IL-10, TNF-alpha, IFN-gamma, IFN-α2a, Gro-a, MCP-1α, and MIP-Iα.

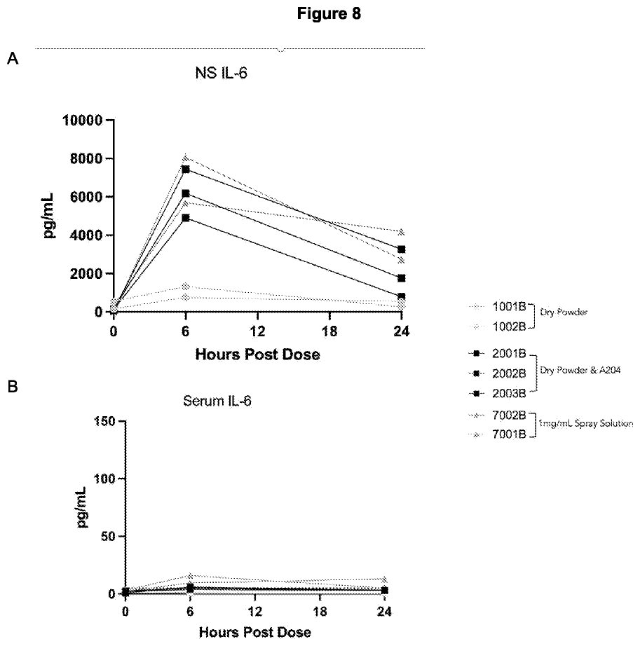

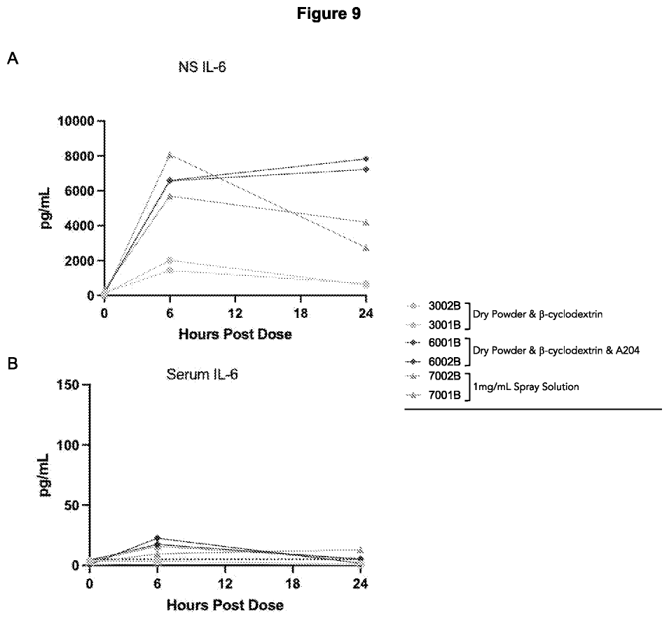

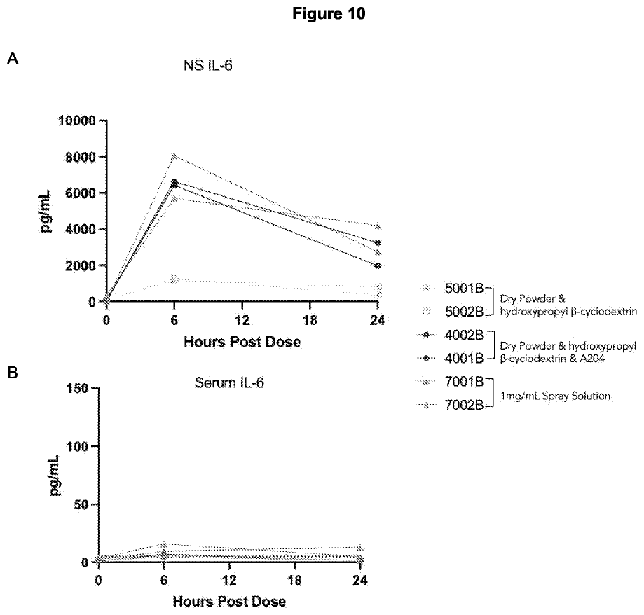

Results of IL-6 analysis are shown in FIGS. 8-10. These data show that the pharmaceutical compositions of the invention (FIGS. 9 and 10) are able to effectively achieve local nasal delivery of the compound comprising a TLR2 agonist moiety conjugated with a solubilising agent. These data show that the formulations are able to desirably retain A204 in the nasal area (comparison of nasal vs serum biomarker levels in FIGS. 8A, 9A and 10A (nasal) compared with FIGS. 8B, 9B and 10B (serum/systemic).

The compositions of the invention comprising a cyclodextrin are shown to improve the storage stability.

However, cyclodextrins are known penetration enhancers, which is not desired for local nasal administration of the TLR2 agonist compounds. The data in this example show that the compositions comprising a cyclodextrin do not result in undesired systemic administration of the TLR2 agonist compound (A204) while providing a bioavailable and pharmaceutically acceptable dosage form with desirable storage stability.

### Example 19—A204 Efficacy Against SARS-CoV-2 in the Hamster Model—Prophylaxis

To demonstrate the efficacy (dose/regimen) of TLR2 agonist-based nasal treatment during TRT SARS-CoV-2 in vivo in hamster model, six hamsters were mock treated with 100 ul PBS 24 hours prior to infection. Animals were infected with 10⁴ PFU SARS-CoV-2 in 100 ul, and weights were taken daily in addition to animal welfare scoring. Animals from all cohorts were throat swabbed to assess viral loads at 2 dpi and culled at 7 dpi.

Nasal tissue and lung tissue were harvested and utilized for RNA extraction and viral enumeration via RT-qPCR.

### Results

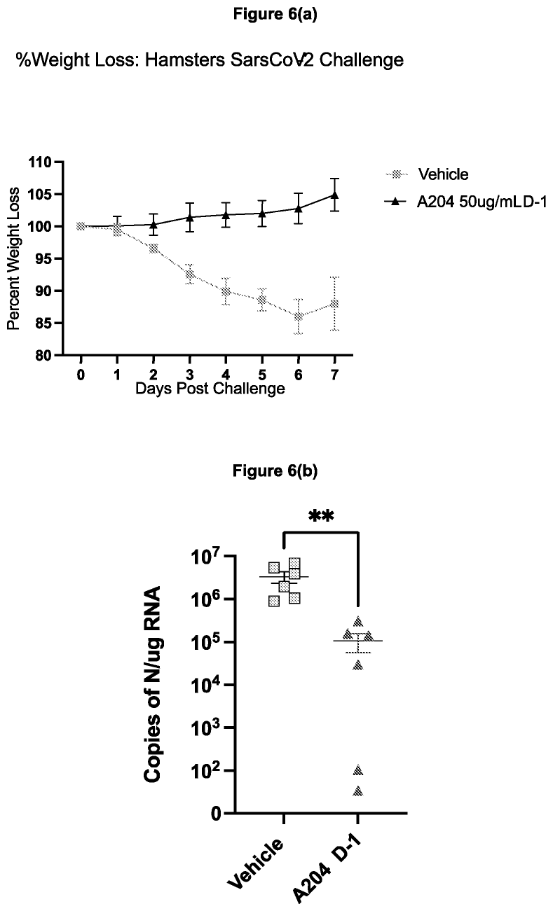

Animals pre-treated with A204 lost no weight during the course of the infection and instead steadily gained weight, indicating that pretreatment with 50 ug/ml conferred protection to the symptoms of SARS-CoV-2 infection (FIG. 6A).

Swabs were taken at day 2 post infection, total RNA extracted using TRIzol reagent and RT-qPCR carried out to quantify viral load. At 2 days post infection the animals exhibited 32-fold lower levels of viral RNA compared to the mock treated animals (FIG. 6B).

At 7 dpi lung viral loads were found to be slightly lower in treated animals compared to the mock treated animals (data not shown).

### Example 20—A204 Efficacy Against SARS-CoV-2 in the Hamster Model—Treatment

Additionally, the inventors sought to investigate the effects of treatment with A204 shortly after SARS-CoV-2 infection. An experiment was undertaken to investigate the potential protective effect of treatment of hamsters with doses of A204 post infection with SARS-CoV-2. Groups (n=10) were infected with 10⁴ PFU SARS-CoV-2 and then treated 8 hours post infection (hpi) with 10 ug/ml A204 in 100 ul.

### Results

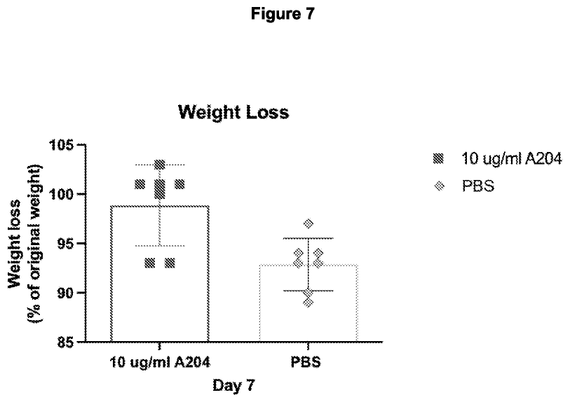

Animals were infected with 10⁴ PFU SARS-CoV-2 and treated 8 hours post infection with 1 ug/100 ul (10 ug/ml) A204 or PBS. The group treated with 10 ug/ml recovered weight loss more rapidly than PBS treated and by 7 dpi were essentially back to original weight whereas PBS treated were not (FIG. 7).

It will be understood that the invention disclosed and defined in this specification extends to all alternative combinations of two or more of the individual features mentioned or evident from the text or drawings. All of these different combinations constitute various alternative aspects of the invention.

1. A pharmaceutical composition comprising:
a cyclodextrin; and
a compound comprising a TLR2 agonist moiety conjugated with a solubilising moiety, or a pharmaceutically acceptable salt, solvate, stereoisomer and/or prodrug thereof.

2. The pharmaceutical composition of claim 1, comprising the compound and the cyclodextrin in a ratio by weight from about 1:1 to about 1:100.

3. The pharmaceutical composition of claim 1, comprising the compound and the cyclodextrin in a ratio by weight from about 1:30 to about 1:99.

4. The pharmaceutical composition of claim 1, comprising the compound in a concentration from about 0.5 wt % to about 5 wt %.

5. The pharmaceutical composition of claim 1, wherein the cyclodextrin is selected from α-cyclodextrin, β-cyclodextrin, γ-cyclodextrin, and a cyclodextrin derivative of α-cyclodextrin, β-cyclodextrin or γ-cyclodextrin, or a combination thereof.

6. The pharmaceutical composition of claim 5, wherein the cyclodextrin derivative is selected from hydroxypropyl-β-cyclodextrin, sulfobutylether-β-cyclodextrin, methyl-β-cyclodextrin, dimethyl-β-cyclodextrin, and randomly methylated-β-cyclodextrin, or a combination thereof.

7. The pharmaceutical composition of claim 1, further comprising a pharmaceutically acceptable excipient.

8. The pharmaceutical composition of claim 7, wherein the pharmaceutically acceptable excipient comprises a sugar compound selected from mannitol, erythritol, xylitol, sorbitol, myo-inositol or a combination thereof.

9. The pharmaceutical composition of claim 7, wherein the pharmaceutically acceptable excipient comprises a polymer selected from methylcellulose, hydroxypropylmethylcellulose, polyvinyl pyrrolidone and combinations thereof.

10. The pharmaceutical composition of claim 1, wherein the solubilising moiety comprises a polyethylene glycol.

11. The pharmaceutical composition of claim 1, wherein the TLR2 agonist moiety comprises a lipopeptide or a lipid moiety.

12. The pharmaceutical composition of claim 1, wherein the TLR2 agonist moiety comprises at least one palmitoyl, myristoyl, stearoyl, lauroyl, octanoyl, or decanoyl group.

13. (canceled)

14. The pharmaceutical composition of claim 1, wherein the compound is a compound of formula (IA2):
A-Y-B (IA2)
wherein A comprises or consists of: 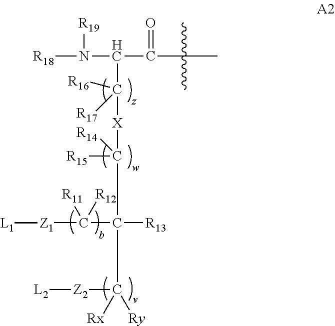
wherein
b and w are each independently an integer from 0 to 7 and v is an integer from 0 to 5, such as from 2 to 5, provided that:
the sum of b, v, and w is at least 3; and
the sum of b and w is from 0 to 7;
z is 1 or 2;
X is selected from —S—, —S(═O)— and —S(═O)₂—;
Z₁ and Z₂ are each independently selected from the group consisting of —O—, —NR—, —S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A2;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
Y is 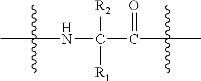
wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl; and
B comprises or consists of Polyethylene Glycol (PEG),
or a pharmaceutically acceptable salt, solvate or prodrug thereof.

15. The pharmaceutical composition of claim 1, wherein the compound is a compound of formula (XX): 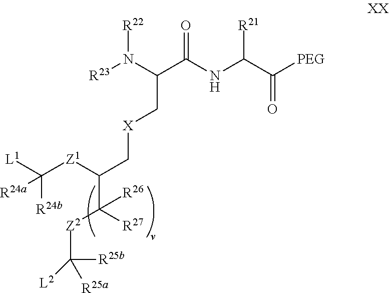
wherein:
R²¹ is selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R²² is H, C₁-C₆ aliphatic, an amino protecting group, L³-C(═O)—, or A²;
L¹ and L² are each independently C₆-C₂₁ aliphatic or C₅-C₂₀ heteroaliphatic;
L³ is C₁-C₂₁ aliphatic or C₂-C₂₀ heteroaliphatic;
A² is an amino acid or a peptide;
R²³ is H or C₁-C₆ aliphatic;
R^{24a} and R^{25a} are each independently selected from C₁-C₆ aliphatic and C₁-C₆ heteroaliphatic and R^{24b} and R^{25b} are each independently selected from H, C₁-C₆ aliphatic and C₁-C₆ heteroaliphatic, or
R^{24a} and R^{24b} together with the carbon atom to which they are attached form a C₃₋₈cycloalkyl or 3-8 membered heterocyclyl group, and/or
R^{25a} and R^{25b} together with the carbon atom to which they are attached form a C₃₋₈cycloalkyl or 3-8 membered heterocyclyl group;
X is selected from —S—, —S(═O)— and —S(═O)₂—;
v is an integer from 1-3
R²⁶ and R²⁷ are each independently selected from H and C₁-C₆ aliphatic;
Z¹ and Z² are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
PEG is a polyethylene glycol;
wherein any aliphatic, heteroaliphatic, cycloalkyl and heterocyclyl present in any of R²¹, R²², R²³, R^{24a}, R^{24b}, R^{25a}, R^{25b}, R²⁶, R²⁷, L¹, L² and L³ is optionally substituted.
or a pharmaceutically acceptable salt, solvate, stereoisomer or prodrug thereof.

16. The pharmaceutical composition of claim 14, wherein the polyethylene glycol is a substituted polyethylene glycol represented by partial formula B-I: 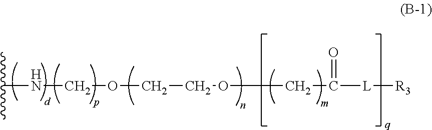
wherein
n is 3 to 100;
m is 1, 2, 3 or 4;
p is 2, 3 or 4;
q is null or 1;
d is null or 1;
R₃ is H, —NH₂ or —OH, wherein when q is null, R₃ is H and when q is 1, R₃ is —NH₂ or —OH;
L is null or consists of 1 to 10 units, wherein each unit is a natural alpha amino acid or derived from a natural alpha amino acid, and has the formula: 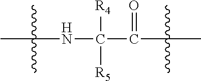
wherein R₄ is H; and
R₅ is the side chain, or second hydrogen of the amino acid.

17. The pharmaceutical composition of claim 1, wherein the compound is any one of the following compounds:

| | Compound |
|---|---|
| Compound Structure | name |
| 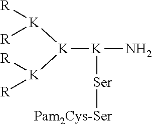 | 001 |
| 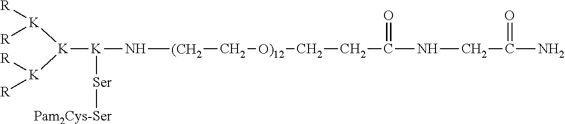 | 002 |
| 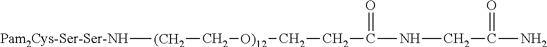 | 003 |
| Pam₂Cys-Ser-Ser-Lys-Lys-Lys-Lys | 004 |
| Pam₂Cys-Ser-Lys-Lys-Lys-Lys | 005 |
| 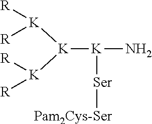 | 007 |
| 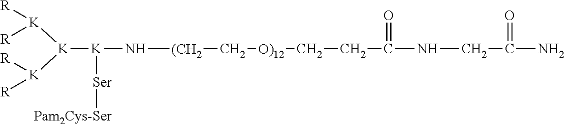 | 008 |
| 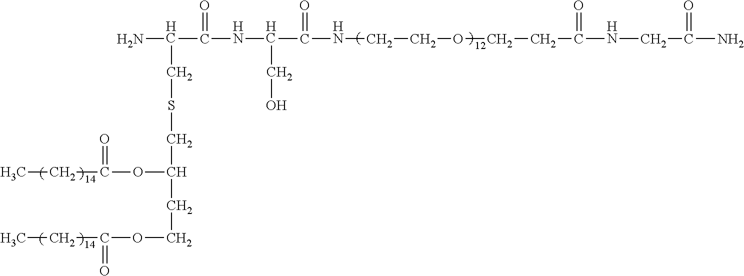 | A201 |
| 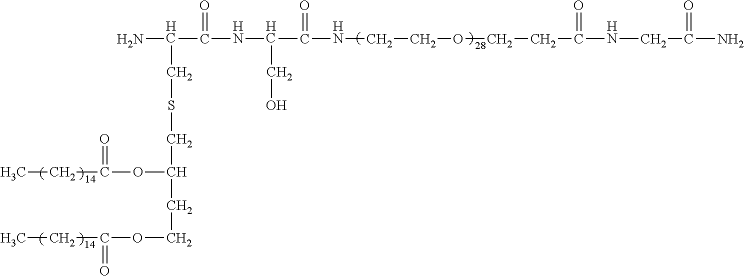 | A202 |
| 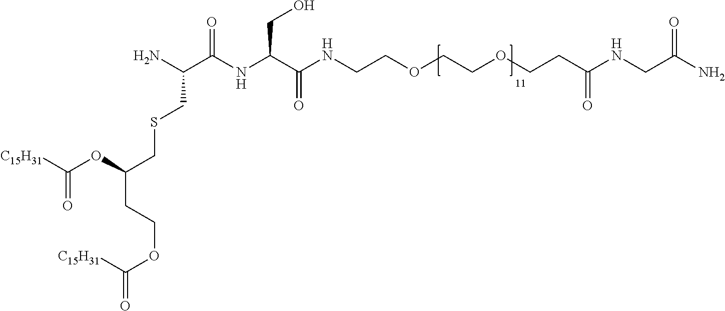 | A203 |
| 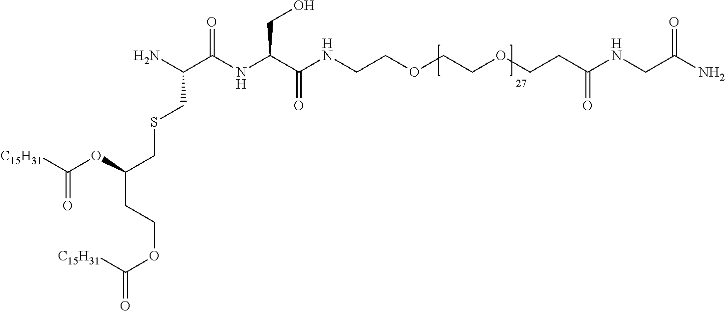 | A204 |
| 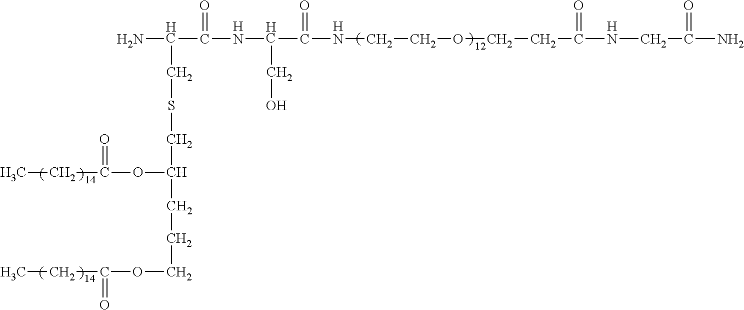 | A205 |
| 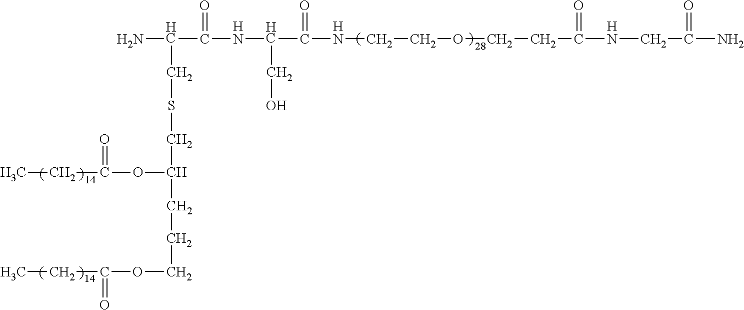 | A206 |
| 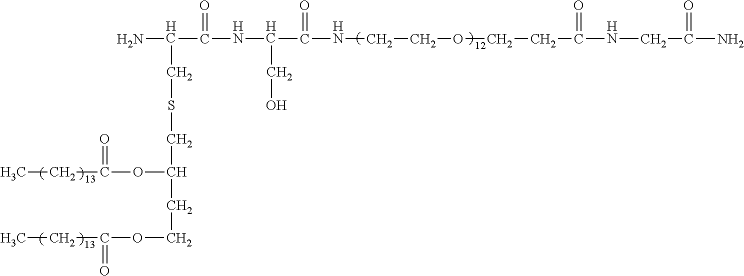 | A207 |
| 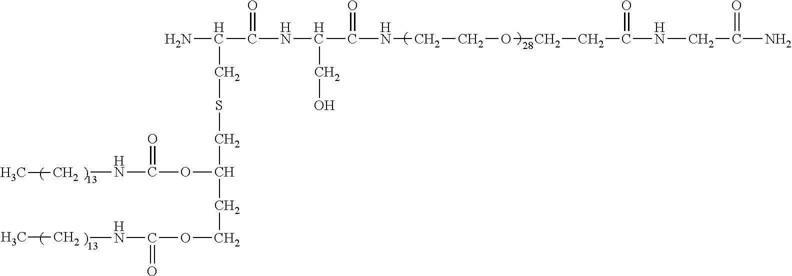 | A208 |
| 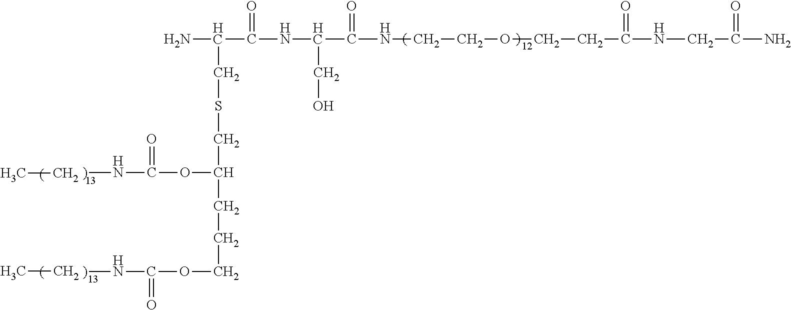 | A209 |
| 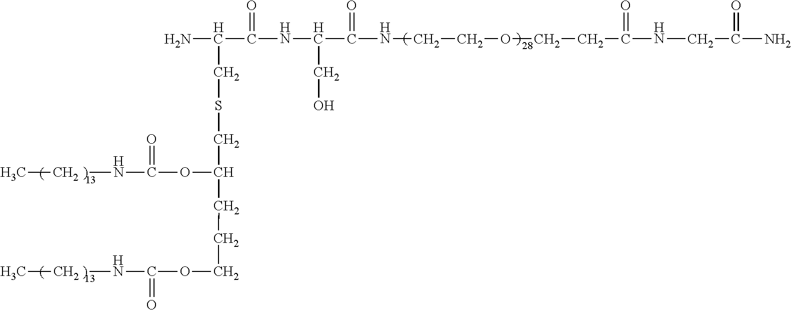 | A210 |
| 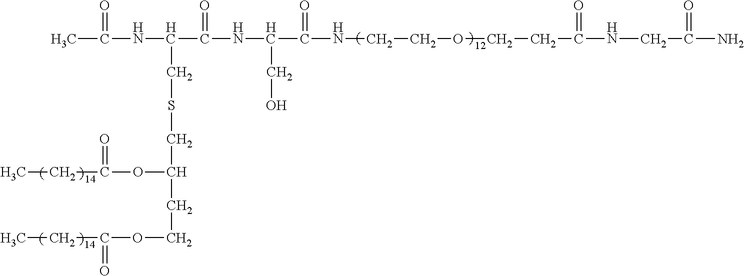 | A211 |
| 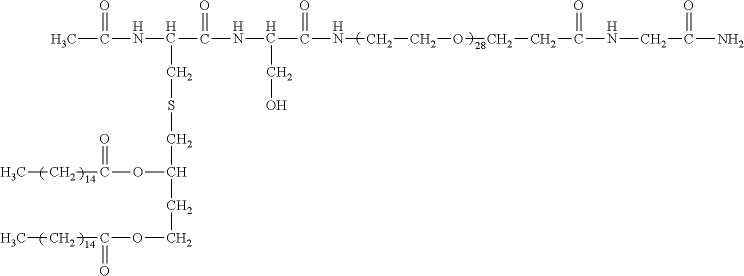 | A212 |
| 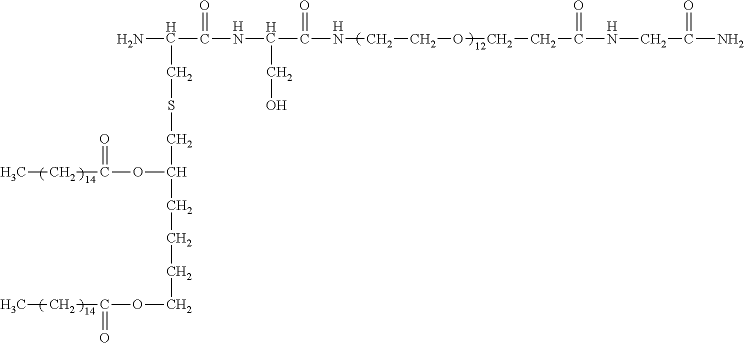 | A213 |
| 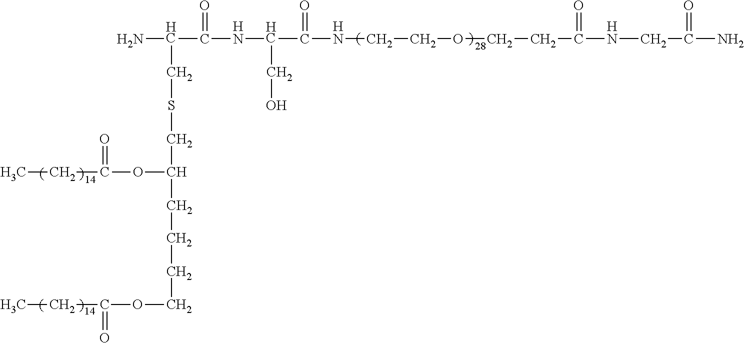 | A214 |
| 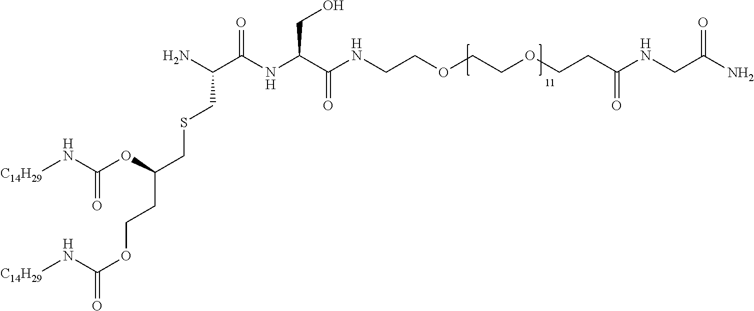 | A215 |
| 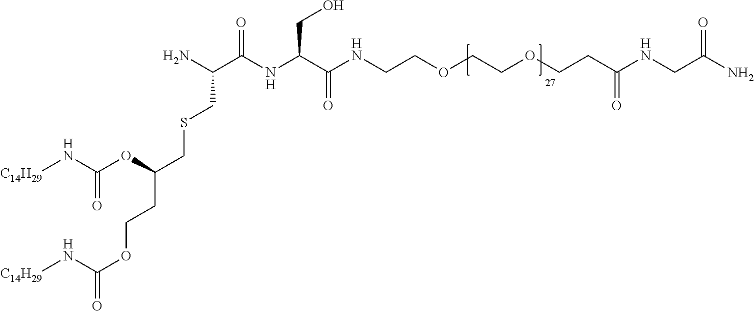 | A216 |
| 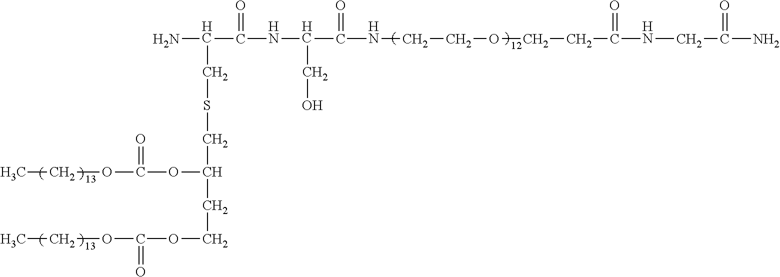 | A217 |
| 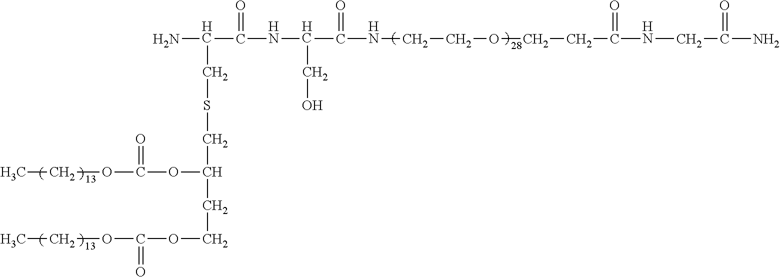 | A218 |
| 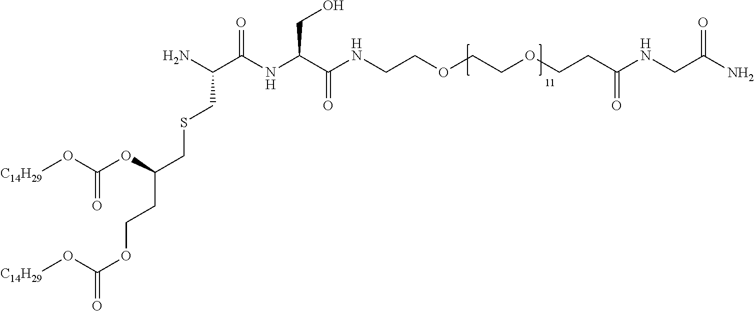 | A219 |
| 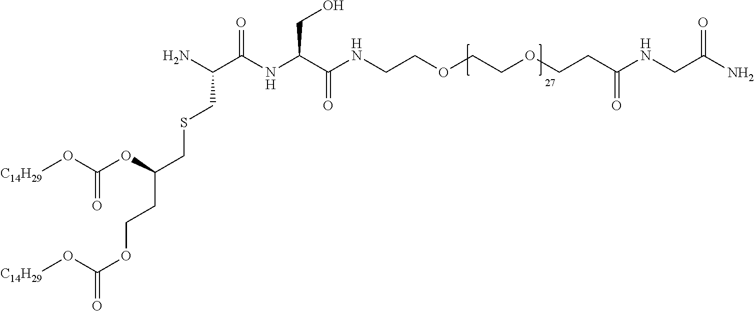 | A220 |
| 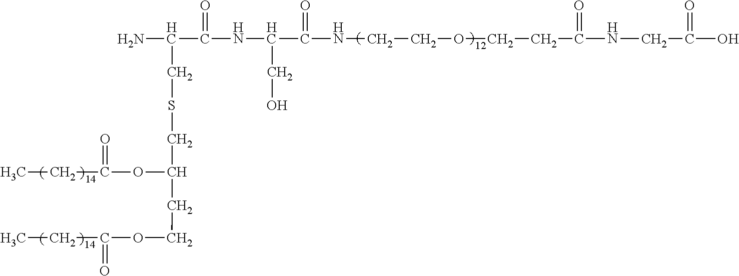 | A221 |
| 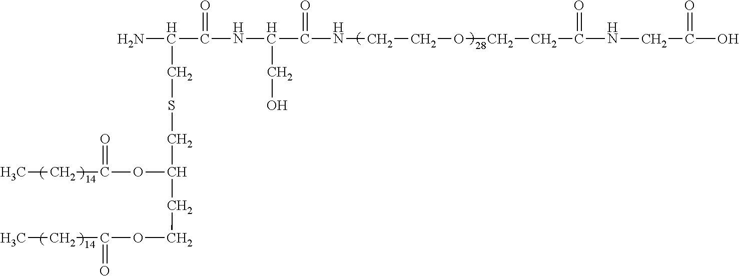 | A222 |
| 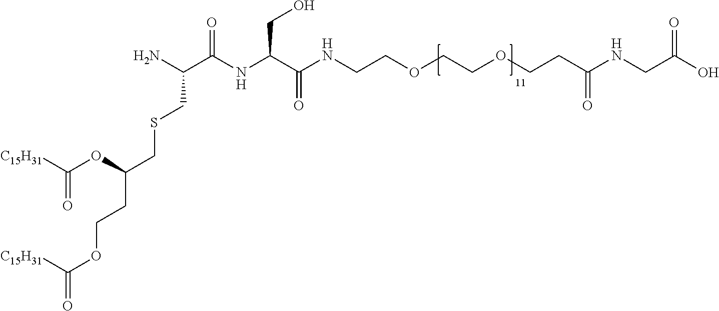 | A223 |
| 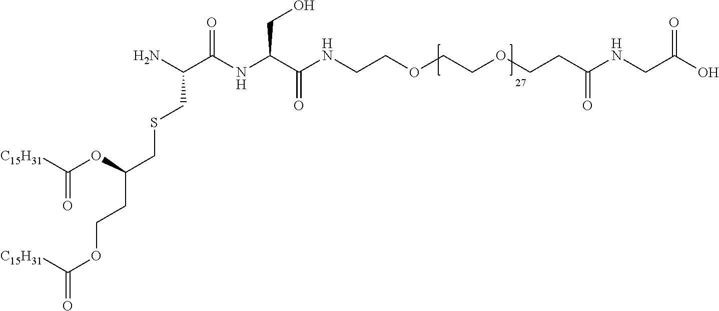 | A224 |
| 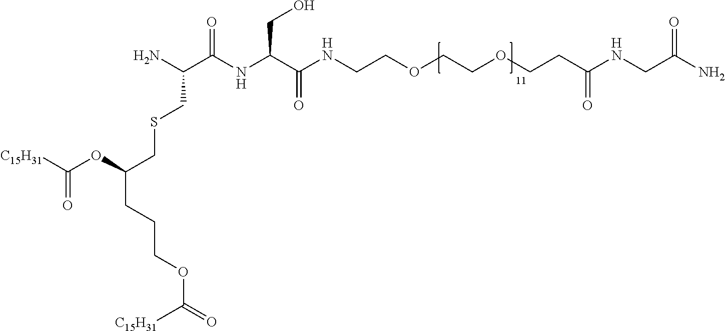 | A225 |
| 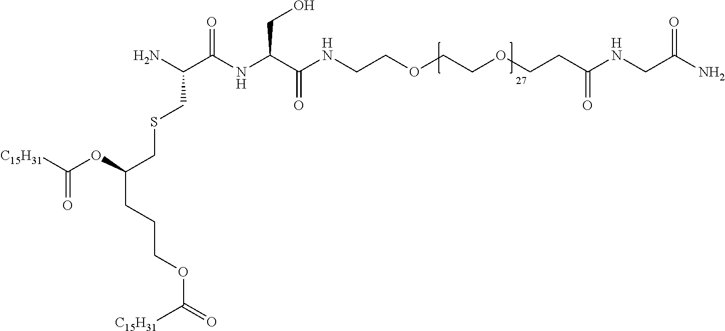 | A226 |
| 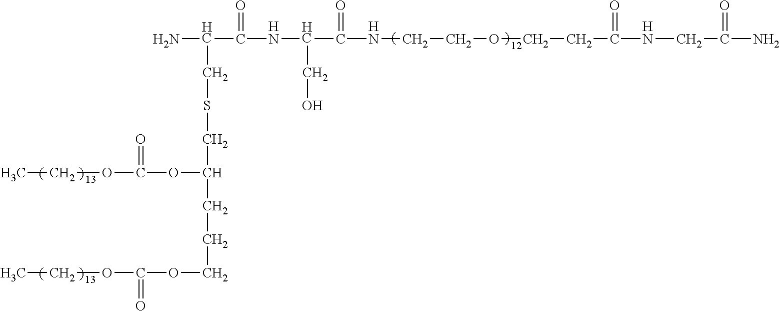 | A227 |
| 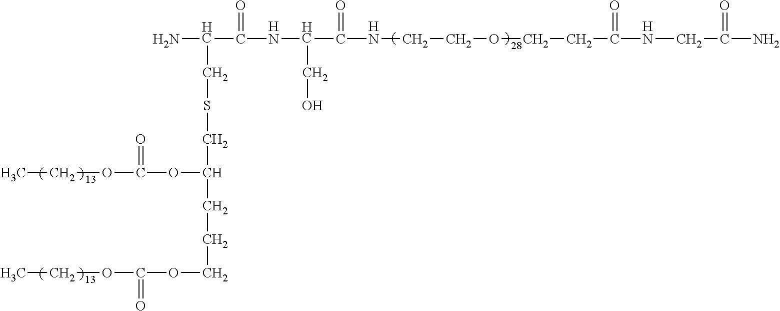 | A228 |
| 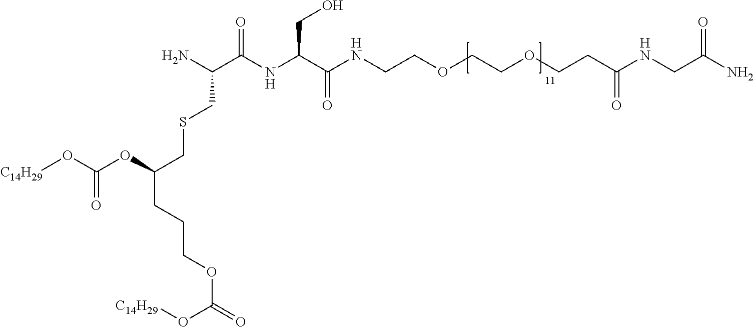 | A229 |
| 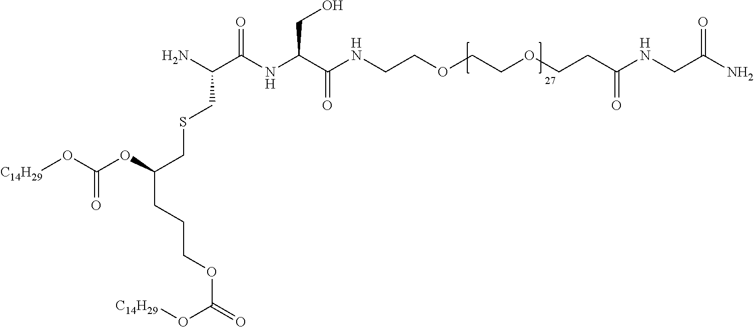 | A230 |
| 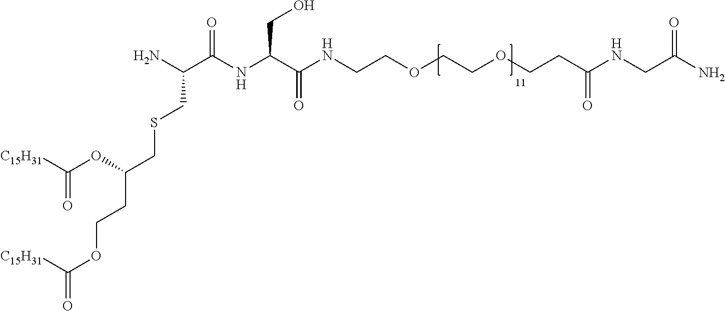 | A231 |
| 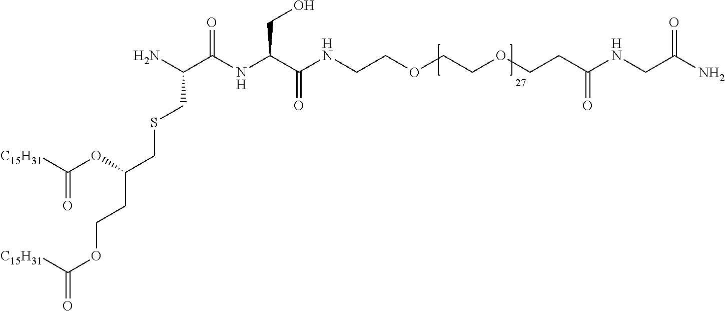 | A232 |
| 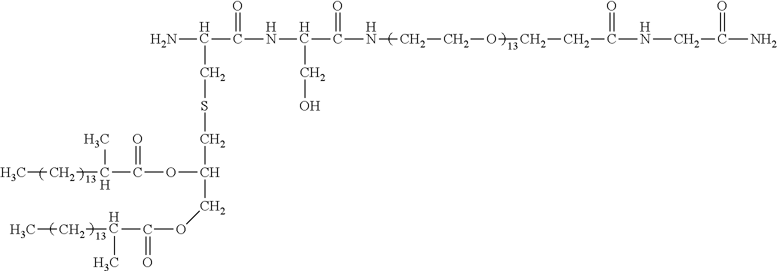 | B1 |
| 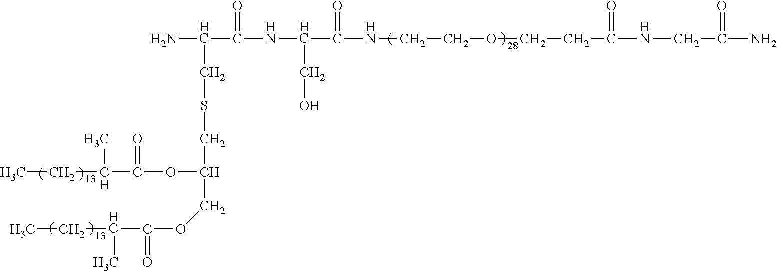 | B2 |
| 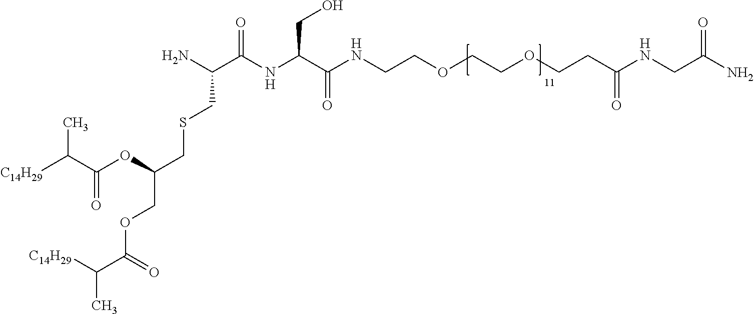 | B3 |
| 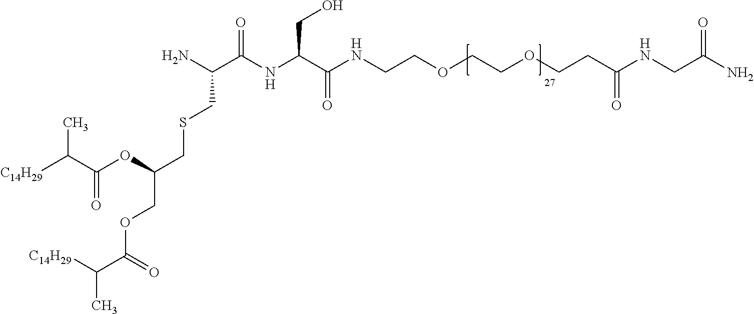 | B4 |
| 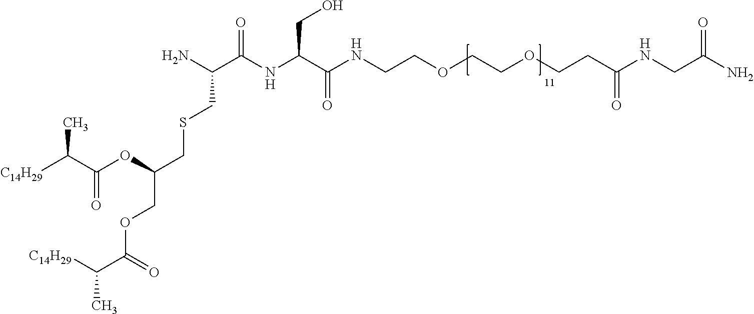 | B5 |
| 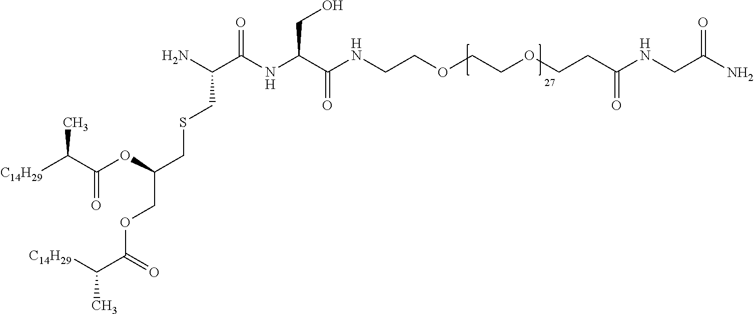 | B6 |
| 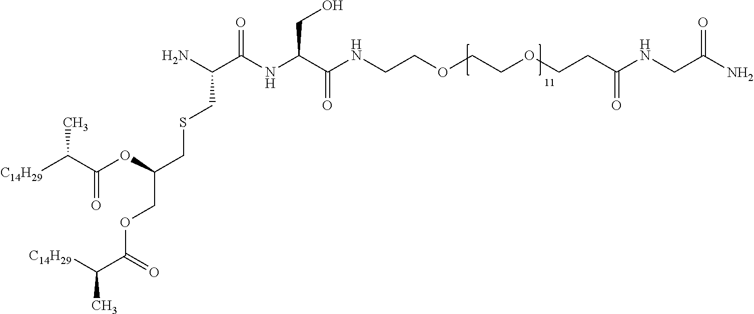 | B7 |
| 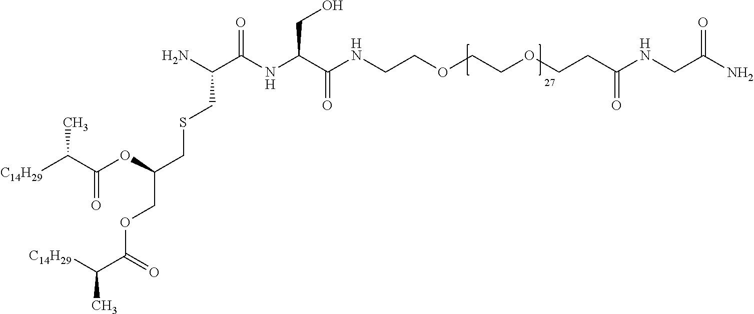 | B8 |
| 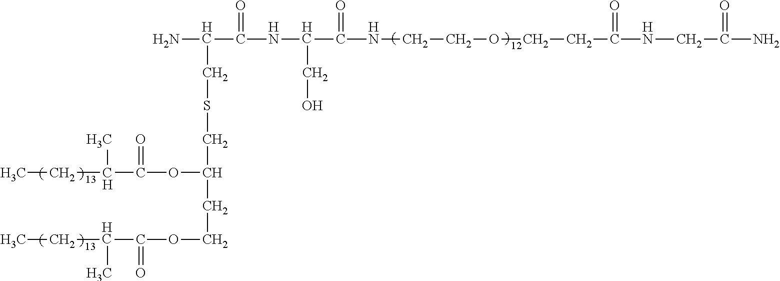 | B9 |
| 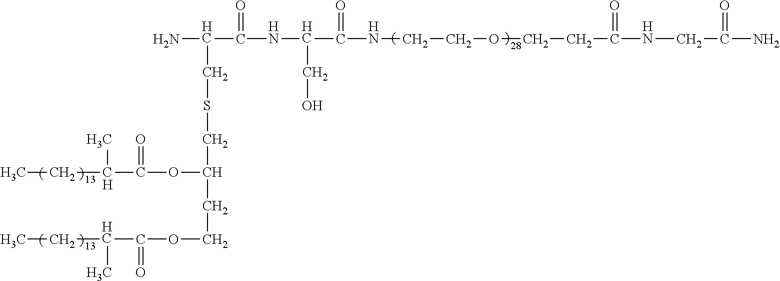 | B10 |
| 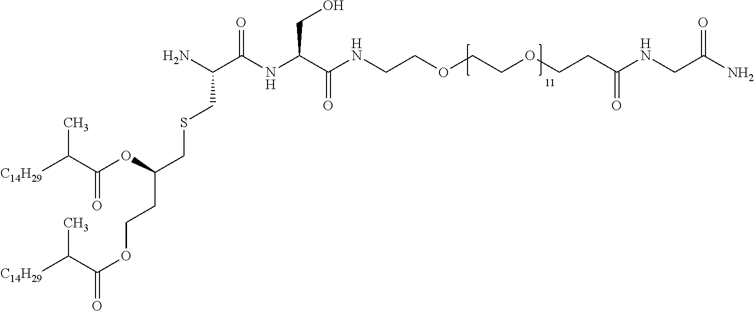 | B11 |
| 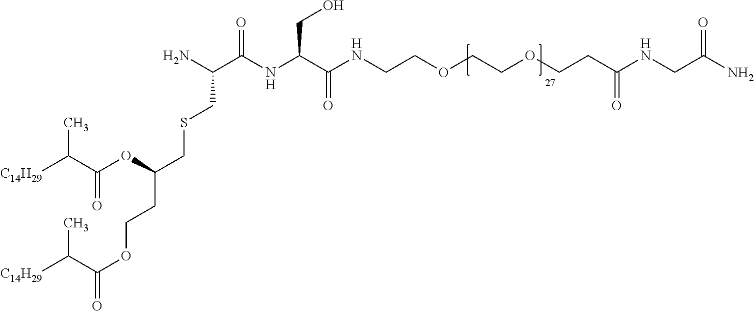 | B12 |
| 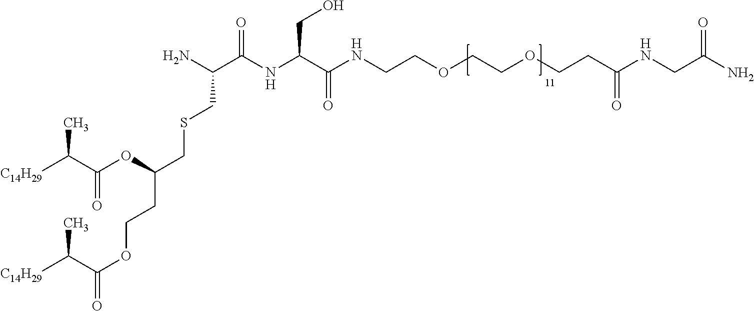 | B13 |
| 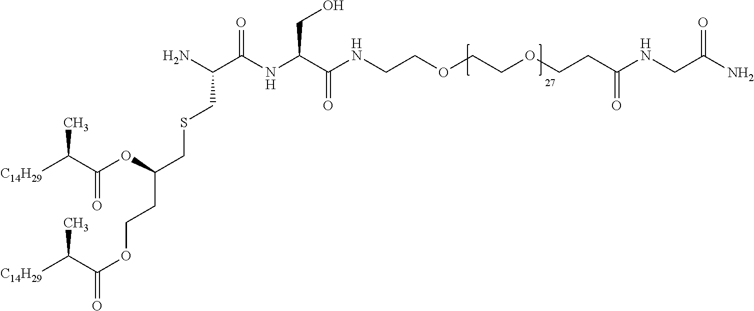 | B14 |
| 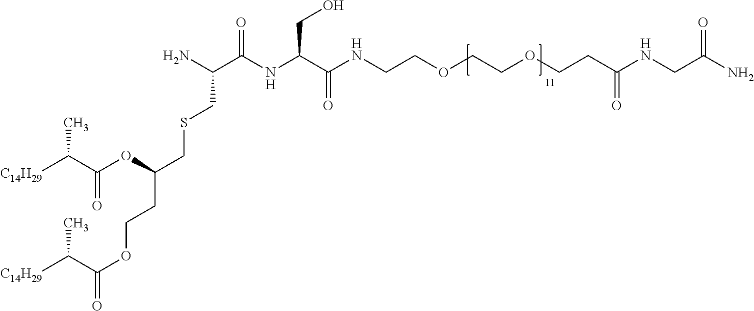 | B15 |
| 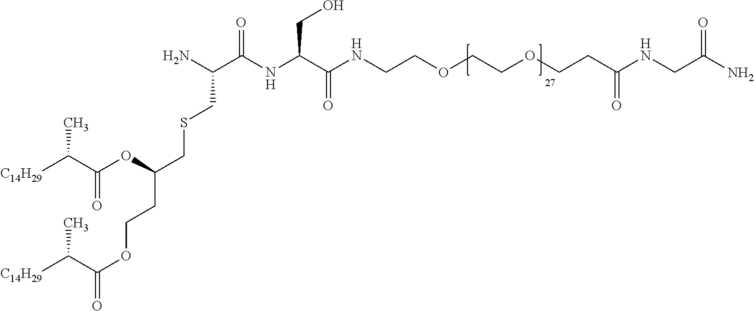 | B16 |

or a pharmaceutically acceptable salt, solvate, stereoisomer or prodrug thereof.

19. The pharmaceutical composition of claim 1, in the form of a powder or in the form of a solution.

20-22. (canceled)

23. A method of preparing a powder comprising a compound comprising a TLR2 agonist moiety conjugated with a solubilising moiety, or a pharmaceutical acceptable salt, solvate, stereoisomer or prodrug thereof, and a cyclodextrin, the method comprising:
forming a solution comprising the compound and the cyclodextrin; and
spray drying the solution to provide the powder.

24. A method comprising administering an effective amount of the pharmaceutical composition of claim 1 to a subject in need thereof, wherein the method comprises one or more of the following:
(a) raising an innate immune response in the subject; and/or
(b) treating and/or preventing a disease caused by an infectious agent; and/or
(c) treating and/or preventing a respiratory disease or condition associated with a viral or bacterial infection; and/or
(d) treating and/or preventing a respiratory infection; and/or
(e) reducing airway inflammation; and/or
(f) improving the ability of a subject to control a respiratory disease or condition during a respiratory viral infection; and/or
(g) treating and/or preventing a disease or condition associated with the TLR2 receptor.

25-30. (canceled)
